(19) United States
(12) Patent Application Publication
(10) Pub. No.: US 2019/0264267 A1
(43) Pub. Date: Aug. 29, 2019

(54) PHASING

(71) Applicants: Hailin Yang, Cambridge, MA (US); Aaron Jay Morris, Cambridge, MA (US); Vinod Vathipadiekal, Cambridge, MA (US); WAVE LIFE SCIENCES LTD., Singapore (SG)

(72) Inventors: Hailin YANG, West Roxbury, MA (US); Aaron Jay MORRIS, Brighton, MA (US); Vinod VATHIPADIEKAL, Stoneham, MA (US)

(73) Assignee: Wave Life Sciences Ltd., Singapore (SG)

(21) Appl. No.: 16/320,379

(22) PCT Filed: Jul. 24, 2017
(86) PCT No.: PCT/US2017/043431

§ 371 Date: Jan. 24, 2019

Related U.S. Application Data
(60) Provisional application No. 62/366,585, filed on Jul. 25, 2016.

(51) Int. Cl. C12Q 1/6827; C12Q 1/6853; C12Q 1/6858; C12Q 1/686

(52) U.S. Cl. CPC C12Q 1/6827; C12Q 1/6853; C12Q 1/6858; C12Q 1/686; C12Q 2600/112; C12Q 2563/159; C12Q 2563/179; C12Q 2600/156; C12Q 2600/16; C12Q 2525/161

(57) ABSTRACT

Among other things, the present disclosure pertains to methods and compositions related to phasing of allelic variants of genetic loci. Phasing of allelic variants of genetic loci on an individual patient's chromosomes is highly valuable for many purposes, including patient stratification for allele-specific therapeutics.

### BACKGROUND

Phasing, e.g., of allelic variants of genetic loci on chromosomes is useful for many purposes. There is a need for more efficient methods of phasing, e.g., allelic variants of genetic loci.

### SUMMARY

Among other things, present disclosure encompasses the recognition of the sources of problems with many technologies for phasing.

Sequence and genotype data often identify alleles that are present in a diploid genome without revealing their arrangement as haplotypes. Phasing, e.g., the determination of the arrangement or linkages of the allelic variants of genetic loci on particular chromosomes, is often important for genetic analysis and for fully exploiting the potential of techniques such as genome engineering and allele-specific expression analysis. Phasing of the allelic variants of genetic loci on an individual patient's chromosomes is highly valuable for patient stratification for allele-specific therapeutics. Many technologies for phasing allelic variants of genetic loci can suffer from technical and other limitations.

The present disclosure, among other things, provides technologies (e.g., compositions, methods, etc.) for phasing, e.g., allelic variants of genetic loci on a nucleic acid, such as a chromosome, chromosomal fragment, genomic DNA, mRNA or cDNA, etc. In some embodiments, the present disclosure provides compositions and methods pertaining to nucleic acids comprising two or more genetic loci. These provided nucleic acids are useful for many purposes, including, but not limited to, phasing.

In some embodiments, the present disclosure appreciates that some sources of problems with prior technologies related to phasing include: the difficulty in phasing variant alleles of genetic loci which are very far apart from each on a chromosome; and the difficulty in phasing genetic loci which are intronic, etc.

In some embodiments, the present disclosure provides a method of phasing allelic variants of a first and a second genetic locus on a nucleic acid template comprising the first and the second genetic locus and a first spacing region between the first and the second genetic locus, the method comprising steps of:

(a) providing a sample comprising one or more types of the nucleic acid template;

(b) partitioning the sample into aliquots so that a plurality of the aliquots contain no more than one type of the template;

(c) generating, from at least one aliquot containing one type of the template, a plurality of nucleic acid polymers based on the template, wherein at least one of the nucleic acid polymers comprises the first and the second genetic locus and a second spacing region that is different from the first spacing region; and

(d) phasing the first and the second genetic locus on the at least one nucleic acid polymer to phase the allelic variants of the first and second genetic loci on the nucleic acid template.

In some embodiments, the present disclosure provides a method of phasing allelic variants of multiple genetic loci on a nucleic acid template comprising the multiple genetic loci and multiple spacing regions between the multiple genetic loci, the method comprising steps of:

(a) providing a sample comprising one or more types of the nucleic acid template;

(b) partitioning the sample into aliquots so that a plurality of the aliquots contain no more than one type of the template;

(c) generating, from at least one aliquot containing one type of the template, a plurality of nucleic acid polymers based on the template, wherein at least one of the nucleic acid polymers comprises the multiple genetic loci and wherein at least one of the multiple spacing regions on the nucleic acid polymer is different from the corresponding spacing region on the nucleic acid template; and

(d) phasing the multiple genetic loci on the at least one nucleic acid polymer to phase the allelic variants of the multiple genetic loci on the nucleic acid template.

In some embodiments, the present disclosure provides a method of phasing allelic variants of a first and a second genetic locus on a nucleic acid template comprising the first and the second genetic locus and a first spacing region between the first and the second genetic locus, the method comprising a step of:

(a) phasing the allelic variants on a nucleic acid polymer in order to phase the allelic variants on the nucleic acid template,

wherein the nucleic acid polymer comprises the first and the second genetic locus, and a second spacing region which is different from the first spacing region between the first and the second genetic locus,

wherein the nucleic acid polymer is generated from an aliquot containing one type of the nucleic acid template, and

wherein the aliquot is generated by partitioning a sample comprising one or more types of the nucleic acid template into aliquots so that a plurality of the aliquots contain no more than one type of the template.

In some embodiments, the present disclosure provides a method of phasing allelic variants of multiple genetic loci on a nucleic acid template comprising the multiple genetic loci and multiple spacing regions between the multiple genetic loci, the method comprising a step of:

(a) phasing the allelic variants on a nucleic acid polymer in order to phase the allelic variants on the nucleic acid template,

wherein the nucleic acid polymer comprises the multiple genetic loci, and wherein one or more of the multiple spacing regions on the nucleic acid polymer is different than one or more of the multiple spacing regions on the nucleic acid template,

wherein the nucleic acid polymer is generated from an aliquot containing one type of the nucleic acid template, and

wherein the aliquot is generated by partitioning a sample comprising one or more types of the nucleic acid template into aliquots so that a plurality of the aliquots contain no more than one type of the template.

In some embodiments, the present disclosure provides a method of generating nucleic acid polymers comprising a first and a second genetic locus, the method comprising steps of:

(a) providing a sample comprising one or more types of the nucleic acid template comprising the first and the second genetic locus and a first spacing region between the first and the second genetic locus;

(b) partitioning the sample into aliquots so that a plurality of the aliquots contain no more than one type of the template; and

(c) generating, from each aliquot containing one type of the template, a plurality of nucleic acid polymers based on the template, wherein at least one of the nucleic acid polymers comprises the first and the second genetic locus and a second spacing region that is different from the first spacing region.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

In some embodiments, the present disclosure provides a method of generating a plurality of nucleic acid polymers, wherein at least one of the polymers comprises a first and a second genetic locus of a nucleic acid template comprising the first and the second genetic locus and a first spacing region between the first and the second genetic locus, the method comprising a step of:

generating from a first aliquot of the nucleic acid template a plurality of nucleic acid polymers based on the template, wherein at least one of the nucleic acid polymers comprises the first and the second genetic locus and a second spacing region that is different from the first spacing region; and wherein:

the first aliquot contains no more than one type of the template; and

at least one second aliquot of the nucleic acid template comprises a different type of the nucleic acid template than the first aliquot.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

In some embodiments, the present disclosure provides a method comprising steps of:

(a) providing a collection of discrete reaction aliquots, at least one of which contains:

a single molecule of a nucleic acid template whose base sequence includes a first sequence element and a second sequence element, wherein the first and second sequence elements are separated from one another in the template nucleic acid by a spacing sequence element comprising a plurality of residues, and further wherein at least one of the first and/or second sequence elements is present in two or more different forms within the set of template nucleic acids present in the collection; and

reagents for amplifying nucleic acids;

(b) incubating the at least one reaction aliquot under conditions and for a time sufficient to generate, within the at least one reaction aliquot, a product nucleic acid in which the first and second sequences, or the complement sequences thereof, are linked to one another and some or all of the residues of the spacing element, or the complement thereof, are absent.

In some embodiments, the method further comprising the step of determining the forms of each of the first and second sequence element on the product nucleic acid, in order to determine the forms of each of the first and second sequence element on the nucleic acid template.

In some embodiments, the present disclosure provides a method comprising:

incubating one or more discrete reaction aliquots of a collection, wherein each reaction aliquot of the collection contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection; and

reagents for amplifying nucleic acids;

under conditions and for a time sufficient to generate, within each discrete reaction aliquot, a product nucleic acid in which the first and second sequences, or the complement sequences thereof, are linked to one another and some or all of the residues of the spacing element, or the complement thereof, are absent.

In some embodiments, the method further comprises the step of determining the forms of each of the first and second sequence element on the product nucleic acid, in order to determine the forms of each of the first and second sequence element on the nucleic acid template.

In some embodiments, the present disclosure provides a composition comprising:

a single molecule of a nucleic acid template whose base sequence includes a first sequence element and a second sequence element, wherein the first and second sequence elements are separated from one another in the template nucleic acid by a spacing sequence element that comprises a plurality of residues;

a plurality of nucleic acid molecules in each of which the first and second sequence elements, or the complement sequences thereof, are linked to one another and some or all of the residues of the spacing sequence element, or the complement thereof, are absent; and

optionally an reagent for amplifying the template nucleic acid,

wherein no molecules other than the nucleic acid template and the plurality of nucleic acid molecules contain both the first and the second sequences.

In some embodiments, the present disclosure provides a composition comprising a collection of discrete compositions, each of which independently contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the first and/or second sequence elements is present in two or more different forms within the set of template nucleic acids present in the collection; and

a plurality of nucleic acid molecules, in each of which the first and second sequences, or the complement sequences thereof, are linked to one another and some or all of the residues of the spacing sequence element, or the complement thereof, are absent; and optionally an reagent for amplifying the template nucleic acid.

In some embodiments, the present disclosure provides a composition comprising a collection of discrete compositions, each of which independently contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection;

a plurality of nucleic acid molecules in each of which the first and second sequences, or the complement sequences thereof, are linked to one another and some or all of the residues of the spacing element, or the complement thereof, are absent; and

optionally an reagent for amplifying the template nucleic acid;

wherein in each composition, no molecules other than the template nucleic acid and the plurality of nucleic acid molecules contain both the first and the second sequences.

In some embodiments, the first and/or second genetic locus or sequence element are selected from: a single nucleotide polymorphism (SNP), a restriction fragment length polymorphisms (RFLP), an amplified fragment length polymorphisms (AFLP), random amplified polymorphic DNA (RAPD), an isozyme, a simple sequence repeat (SSR), a mutation, a genetic lesion, a repeat expansion, a deletion, a truncation, an insertion, an inversion, fusion, or a region of chromosome breakage and/or chromosome breakage and/or re-attachment.

In some embodiments, the nucleic acid template is determined to be heterozygous at the genetic loci.

In some embodiments, the nucleic acid template is determined to be heterozygous at the genetic loci, wherein the determination is performed using an PCR based genotyping assay.

In some embodiments, the nucleic acid template in step (a) has been determined to be heterozygous at the genetic loci.

In some embodiments, the nucleic acid template in step (a) has been determined to be heterozygous at the genetic loci using an PCR based genotyping assay.

In some embodiments, a first genetic locus and a second genetic locus independently comprises a characteristic sequence element. In some embodiments a characteristic sequence element comprises a SNP, a RFLP, an AFLP, an isozyme, a SSR, a mutation, a genetic lesion, a SNP, a repeat expansion, a deletion, a truncation, an insertion, an inversion, fusion, or a region of chromosome breakage and/or chromosome breakage and/or re-attachment. In some embodiments a characteristic sequence element is a SNP, a RFLP, an AFLP, an isozyme, a SSR, a mutation, a genetic lesion, a SNP, a repeat expansion, a deletion, a truncation, an insertion, an inversion, fusion, or a region of chromosome breakage and/or chromosome breakage and/or re-attachment. In some embodiments, a first genetic locus is or comprises an SNP, and a second genetic locus comprises a characteristic sequence element related to a disease. In some embodiments, a disease is Huntington's disease. In some embodiments a characteristic sequence element is CAG repeats.

In some embodiments, a first genetic locus is at least 5, 10, 15, 20, 25, 30, 40, 50, 60, 70, 80, 90, 100, 200, 300, 500, 1000-base pair in length. In some embodiments, a second genetic locus is at least 5, 10, 15, 20, 25, 30, 40, 50, 60, 70, 80, 90, 100, 200, 300, 500, 1000-base pair in length. In some embodiments, each of a first and second loci is independently at least 5, 10, 15, 20, 25, 30, 40, 50, 60, 70, 80, 90, 100, 200, 300, 500, 1000-base pair in length. In some embodiments, each of a first and second loci is independently at least 20-base pair in length. In some embodiments, each of a first and second loci is independently at least 25-base pair in length. In some embodiments, each of a first and second loci is independently at least 30-base pair in length. In some embodiments, each of a first and second loci is independently at least 40-base pair in length. In some embodiments, each of a first and second loci is independently at least 50-base pair in length. In some embodiments, each of a first and second loci is independently at least 100-base pair in length.

In some embodiments a first spacing region is at least 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 150 kb, 200 kb, 250 kb, 300 kb, 350 kb, 400 kb, 450 kb, 500 kb, 600 kb, 700 kb, 800 kb, 900 kb, 1,000 kb, 2,000 kb, 5,000 kb, or 10,000 kb in length. In some embodiments, a second spacing region is less than 1 kb, 2 kb, 3 kb, 4 kb, 5 kb, 6 kb, 7 kb, 8 kb, 9 kb, 10 kb in length. In some embodiments a second spacing region is at least 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 150 kb, 200 kb, 250 kb, 300 kb, 350 kb, 400 kb, 450 kb, 500 kb, 600 kb, 700 kb, 800 kb, 900 kb, 1,000 kb, 2,000 kb, 5,000 kb, or 10,000 kb shorter than the first spacing region.

### BRIEF DESCRIPTION OF THE DRAWINGS

FIG. 1. An overall example scheme for phasing of allelic variants of genetic loci, e.g., allelic variants of HTT gene for patient stratification. FIGS. 1 to 9 use a sample from fibroblast cells known to have, on one chromosome, a HTT gene with a wt number of CAG repeats (21) and the SNP rs362307_C (wt); and on the other chromosome, a HTT gene with a mutant number of CAG repeats (66) and the SNP rs362307_T (mutant). This sample was used to confirm the validity of an example phasing technique described herein. In this example, the 1st step is genotyping HTT SNP rs362307. The 2nd step is linkage/phasing analysis.

FIG. 2. Example PCR strategy, which brings two independent PCR fragments together with sticky ends.

FIG. 3. Example HTT WT/SNP phasing step.

FIG. 4. Example PCR results.

FIG. 5. Example Phase-Cloning. Primers including a F1Linker and a R1Linker are annealed, for amplication of only the long/full length fragment. Amplification can be, for example, via NGS (next generation sequencing). Fragments can be NGS bar-coded (for multiplexing).

FIG. 6. Example Bar-coding after Agencourt AMPure XP® PCR purification system (Beckman Coulter, Inc., Brea, Calif.).

FIG. 7. Example 2×150 PE MiSeq™ desktop sequencer (Ilumina, San Diego, Calif.) run: R1 read (forward). Both the 21 CAG sequence and >34 CAG sequence from ND fibroblast cells can be seen. The 34 CAG read is restricted by 150-nt reads; if 2×300 PE sequencing were performed, then the maximum length of a CAG sequence which could be detected would be 84 CAG repeats. The inequal distribution of 2 alleles might be due to differences in PCR amplification efficiency.

FIG. 8. Example 2×150 PE MiSeq™ desktop sequencer (Ilumina, San Diego, Calif.) run: R2 read (reverse). An equal distribution of WT and SNP is seen. The sequences shown in FIGS. 7 and 8 thus confirm that the example assay described herein can be used to phase allelic variants of different genetic loci on chromosomal DNA.

FIG. 9. Example Phase-cloning data. FIGS. 1 to 9 confirmed that example assays can be used for phasing allelic variants of two genetic loci.

FIG. 10. Example scheme for phasing of allelic variants of genetic loci using droplet PCR. Without wishing to be bound by any particular theory, example advantages of this scheme can include: the acceptable use of genomic DNA; the absence of a limit to the distance between genetic loci; the ability to analyze intronic genetic loci; and/or low cost. Multiple genetic loci can be analyzed by multiplexing phasing assays, or by using multiple pairs of primers to simultaneously analyze multiple loci.

FIG. 11. Diagram of the Huntingtin gene. Shown are example SNPs such as SNP1, rs362307; SNP2, rs362331; SNP3, rs2530595; and SNP4, rs7685686.

FIG. 12. An example ARMS assay.

FIG. 13. Example genotyping results for 19 cell lines for Huntingtin SNP2 (rs362331, T or C).

FIG. 14. Example genotyping results for 19 cell lines for Huntingtin SNP3 (rs2530595, G or A).

FIGS. 15A and 15B. Example genotyping results for 19 cell lines for Huntingtin SNP1 (rs362307, G or A).

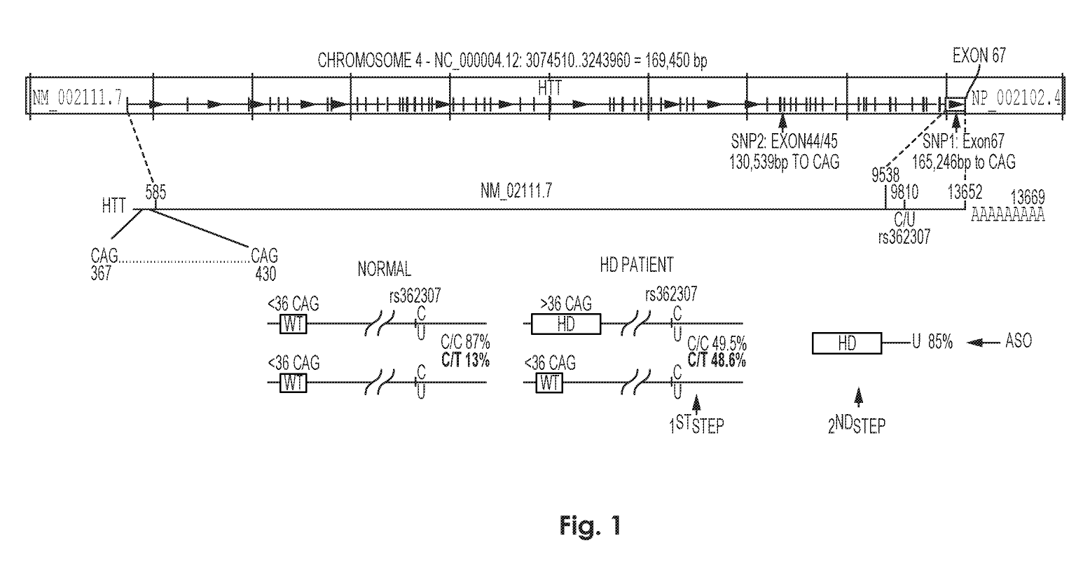

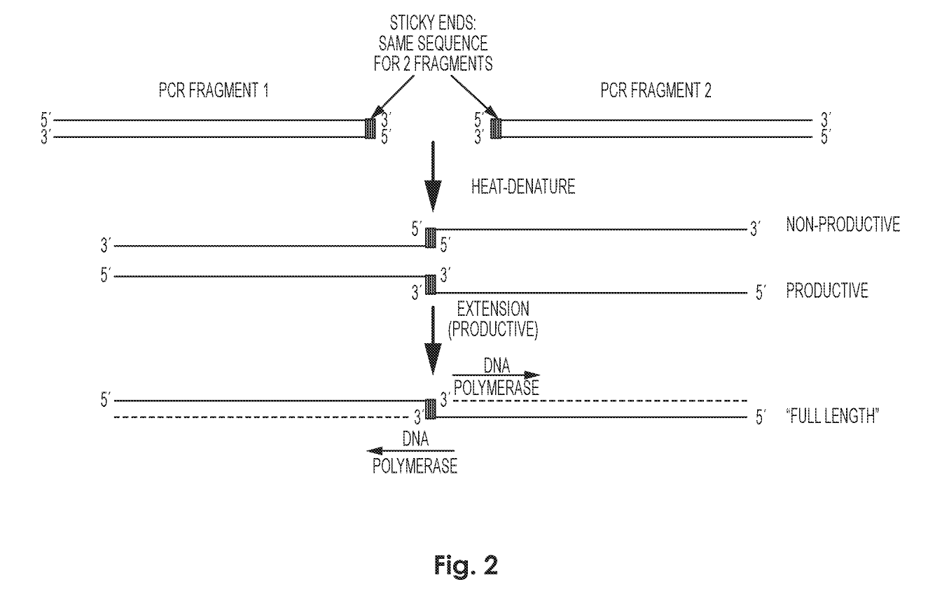

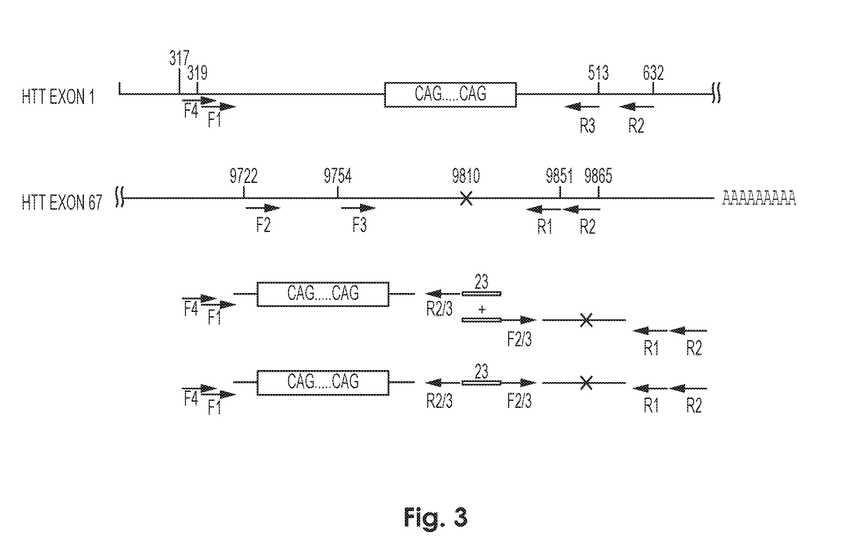

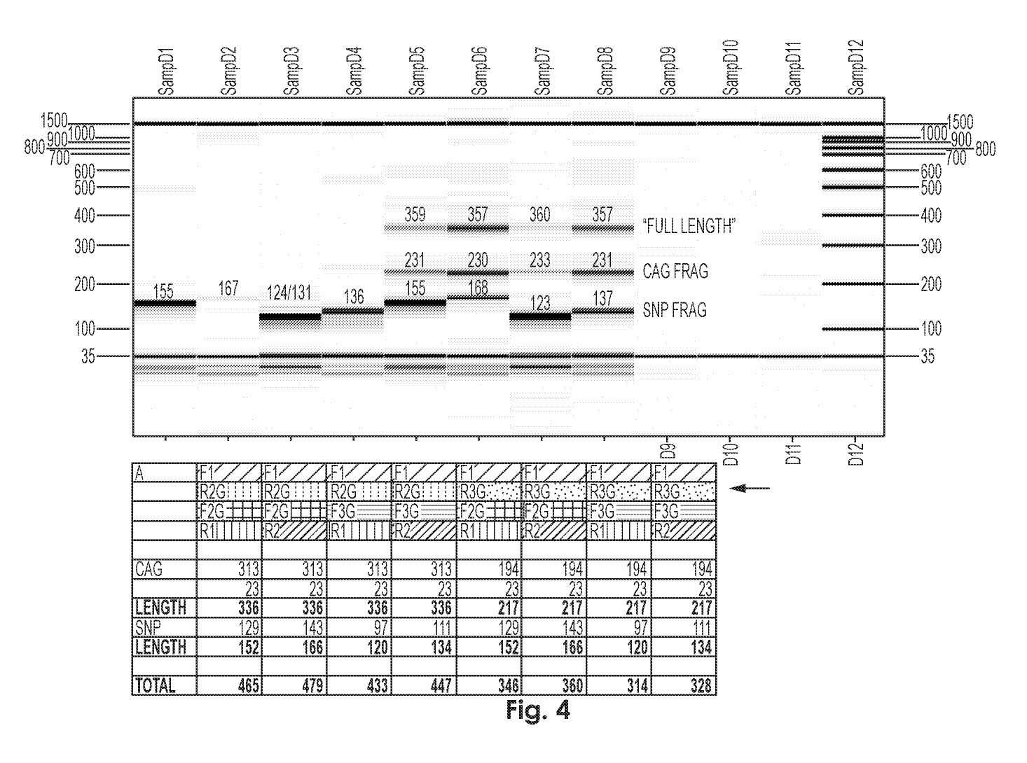

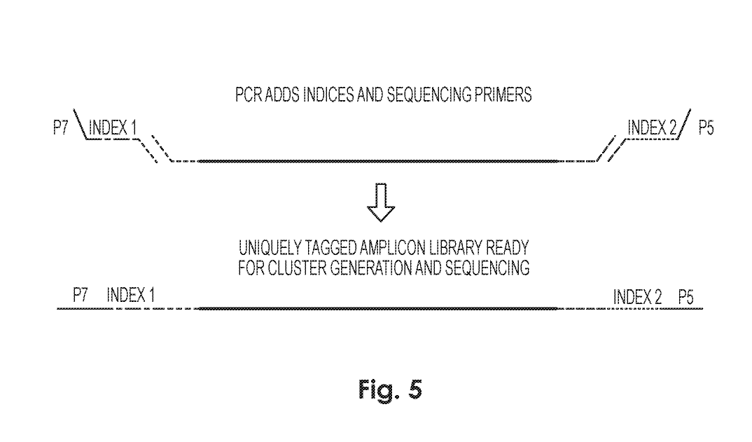

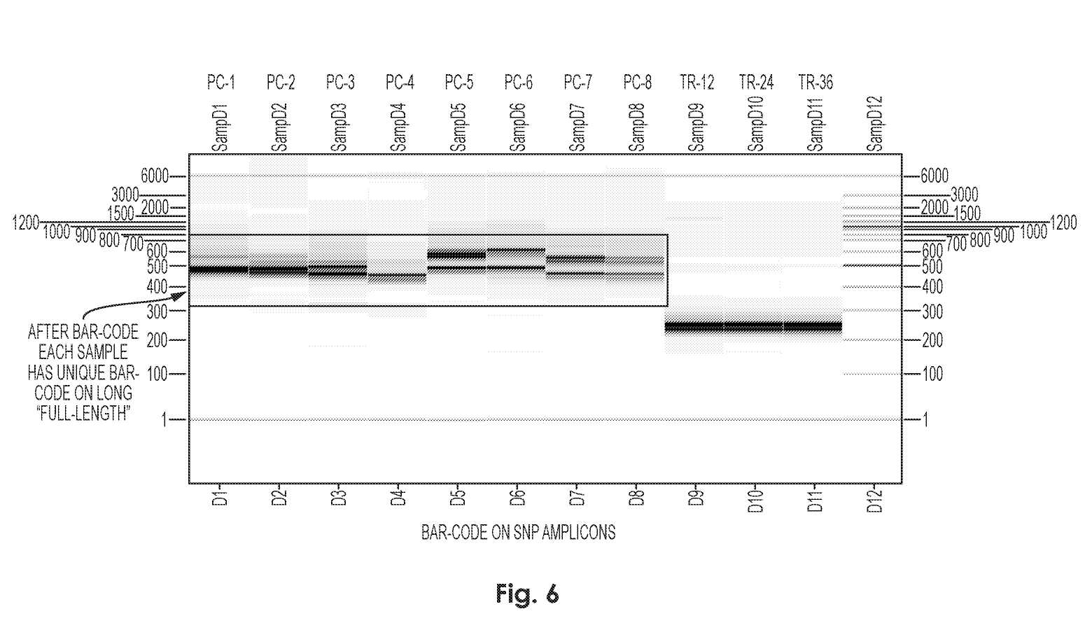

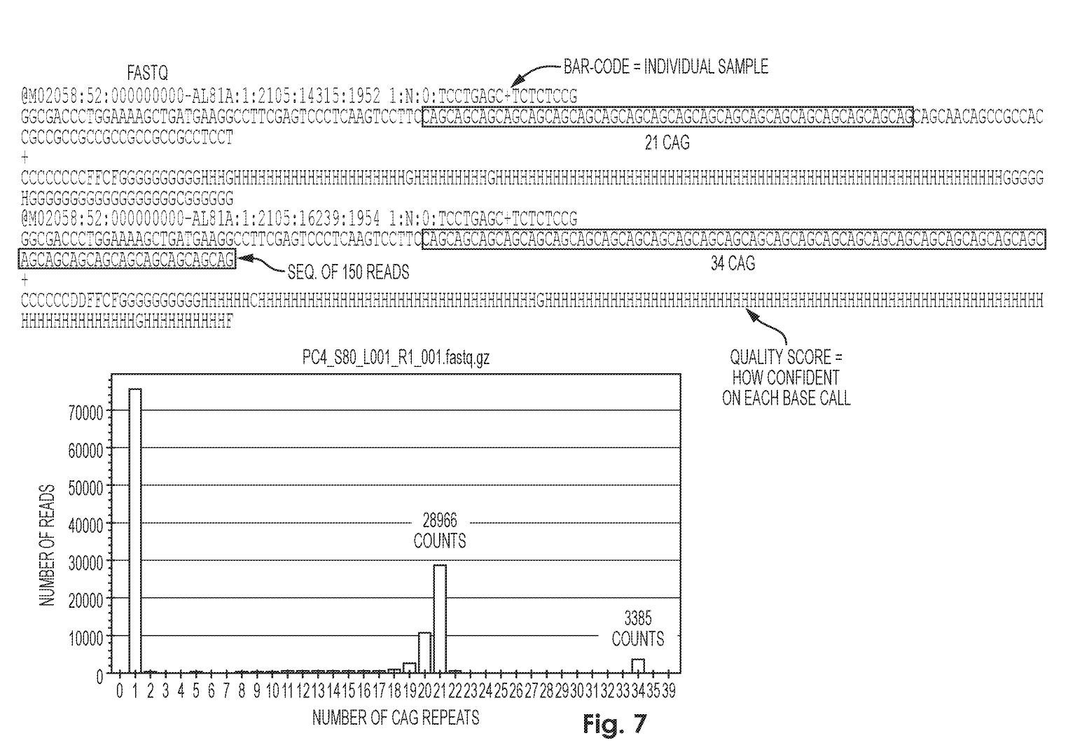

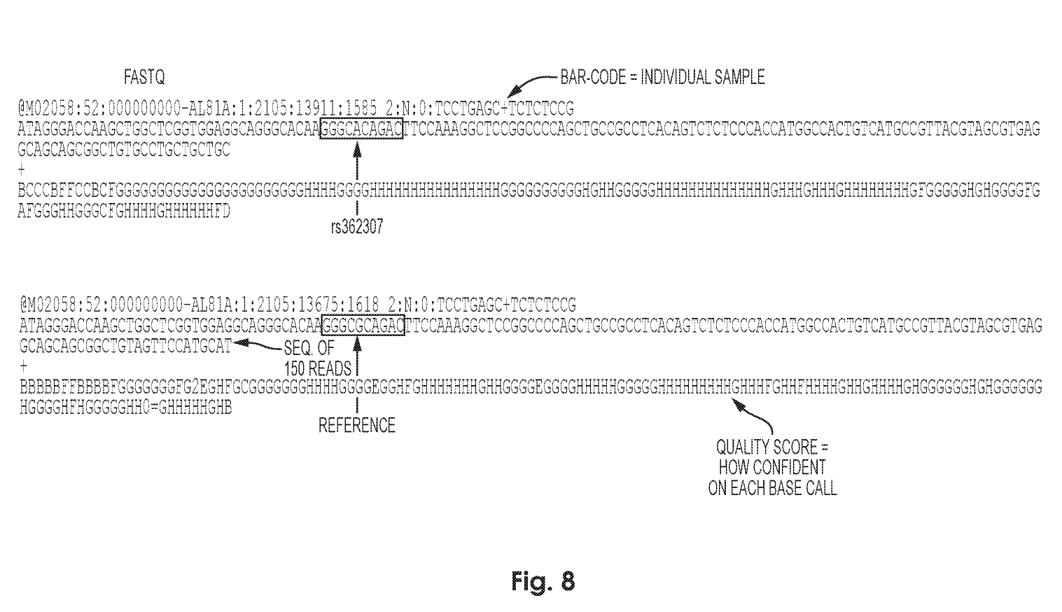

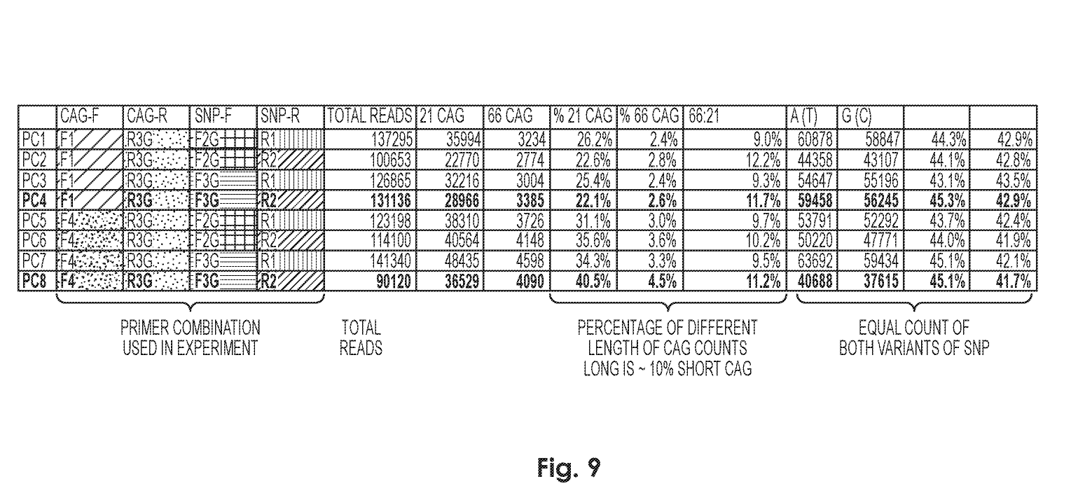

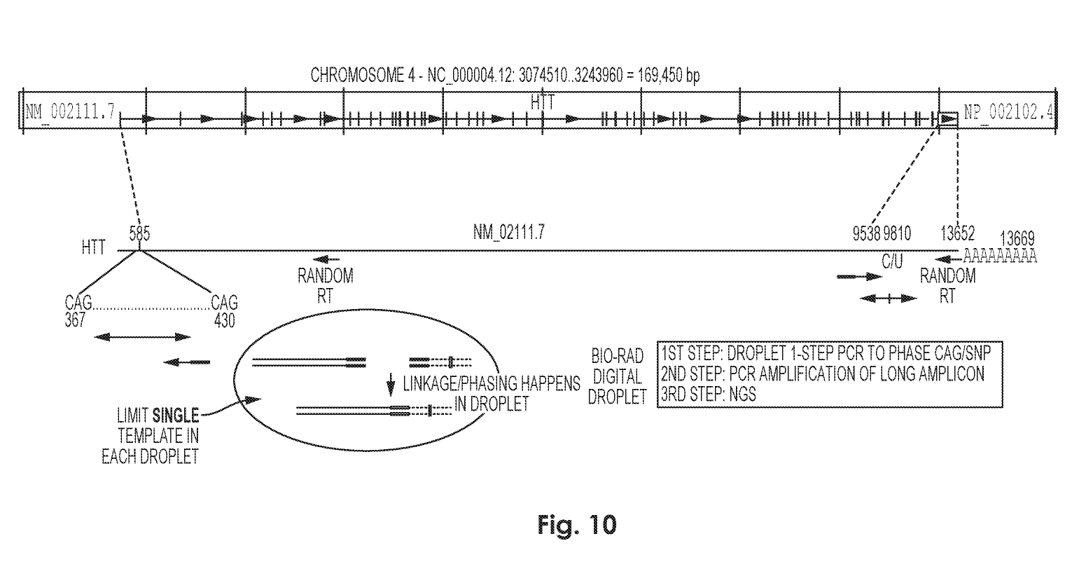

### DETAILED DESCRIPTION OF SOME EMBODIMENTS

### 1. Definitions

Aliquot: The terms “aliquot”, “reaction aliquot”, “composition in a collection of compositions” and the like, as used herein, refers to a portion or a fraction of a whole, e.g., of a sample. In some embodiments, an aliquot represents one of a multitude of fractions of a liquid, semi-liquid or solid sample comprising a nucleic acid template which is intended for analysis by phasing. In some embodiments, a sample (e.g., a biological sample) is divided into a multiple of aliquots. In some embodiments, a sample is diluted and then divided into a multiple of aliquots. In some embodiments, each aliquot comprises on average one or fewer copies of a nucleic acid template. In some embodiments, a sample is divided into aliquots, wherein a plurality of the aliquots contain no more than one type of a template. In some embodiments, aliquots can be droplets, microdroplets, droplets in an emulsion, aqueous droplets in oil, droplets on a solid surface or support, or aliquots in wells covered with oil. In some embodiments, a plurality of aliquots are approximately (±50%) equal in volume. In some embodiments, aliquots have volumes in the range of nanoliters or picoliters. In some embodiments, one or more aliquots are physically separated from others.

Allele, allelic variant and other terms: The terms “allele”, “allelic variant”, “genetic variant” and the like, as used herein, refers to any of several forms (e.g., variant nucleotide sequences) of a genetic locus, sometimes arising through mutation, that are responsible for hereditary variation, e.g., any of two or more nucleotide sequence variants of a genetic locus; these generally have the same relative position on homologous chromosomes and in some cases are responsible for alternative characteristics. For a diploid organism or cell or for autosomal chromosomes, each allelic pair will normally occupy corresponding positions (loci) on a pair of homologous chromosomes, one inherited from the mother and one inherited from the father; this normal positioning may be disturbed in the case of a mutation [e.g., an inversion, fusion (e.g., a gene fusion), deletion, breakage and reattachment of a portion of the chromosome, etc.]. If these alleles are identical, the organism or cell is said to be “homozygous” for that allele; if they differ, the organism or cell is said to be “heterozygous” for that allele. The term “major allele”, as used herein, refers to an allele containing the nucleotide sequence present in a statistically significant proportion (e.g., a plurality or majority) of individuals in the human population. The term “minor allele”, as used herein, refers to an allele containing the nucleotide sequence present in a relatively smaller proportion of individuals in the human population. The terms “wild type allele”, “wt allele” and the like, as used herein, refer to the nucleotide sequence typically not associated with disease or dysfunction of the gene product. The terms “mutant allele” and “mu allele” and the like, as used herein, refer to the nucleotide sequence which differs from the wild type allele; in some cases, the mutant allele is associated with a disease or dysfunction of the gene product. Allelic variants include, inter alia, SNPs or differences between a wild-type and a mutant sequence [such as in inversion, fusion (e.g, a gene fusion), deletion, truncation, substitution, etc.]. The term “variants”, as used herein, refers to substantially similar but non-identical entities, such as nucleotide sequences. As non-limiting examples, for a polynucleotide, a variant can comprise a deletion, inversion, fusion, substitution, and/or addition or other mutation of one or more nucleotides at one or more sites. As used herein, a “native” polynucleotide or polypeptide comprises a naturally occurring nucleotide sequence or amino acid sequence, respectively. One of skill in the art will recognize that variants of the nucleic acids of the embodiments can be constructed such that the open reading frame is maintained. For polynucleotides, conservative variants include variations of a nucleic acid sequence that, because of the degeneracy of the genetic code, do not alter the amino acid sequence of a polypeptide which is encoded. Naturally occurring allelic variants such as these can be identified with the use of well-known molecular biology techniques, as, for example, with polymerase chain reaction (PCR) and hybridization techniques as outlined below.

Amplicon: The term “amplicon”, as used herein, refers to the product of amplification of a nucleic acid template or portion thereof; in some embodiments, an amplicon is a population of polynucleotides, usually double stranded, that are replicated from one or more portions of nucleic acid template. In some embodiments, amplicon is produced by amplification of a portion of a nucleic template (e.g., a chromosome or chromosomal fragment) which has been separated from haplotypes of the nucleic acid template (e.g., homologous chromosomes or fragment thereof), e.g., in various aliquots. Amplicons may be produced by a variety of amplification reactions whose products are multiple replicates of one or more target nucleic acids. Generally, amplification reactions producing amplicon are “template-driven” in that base pairing of reactants, either nucleotides or oligonucleotides, with complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reaction (PCR), linear polymerase reactions, nucleic acid sequence-based amplification (NASBAs), rolling circle amplifications, and the like: Mullis et al, U.S. Pat. Nos. 4,683,195; 4,965,188; 4,683,202; 4,800,159 (PCR); Gelfand et al, U.S. Pat. No. 5,210,015 (real-time PCR with TAQMAN™ probes); Wittwer et al, U.S. Pat. No. 6,174,670; Kacian et al, U.S. Pat. No. 5,399,491 (NASBA); Lizardi, U.S. Pat. No. 5,854,033; Aono et al, Japanese patent publ. JP 4-262799 (rolling circle amplification); and the like. In one aspect, amplicons of the disclosure are produced by PCR. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction.

Animal: The term “animal”, as used herein, refers to a human or non-human animal, including, but not limited to, mice, rats, rabbits, dogs, cats, cows, sheep, pigs, and non-human primates, including, but not limited to, monkeys and chimpanzees.

ARMS Assay: The terms “ARMS assay”, “Amplification Refractory Mutation System”, “allele-specific PCR”, “PCR based genotyping assay” and the like, as used herein, refers to a method of amplification of nucleic acids using allele-specific probes or primers. In some embodiments, an ARMS assay is a low cost and simple method for detecting any mutation, including, as non-limiting examples, those involving single base changes or small deletions.

Autoimmune disease: The term “autoimmune disease,” as used herein, refers to a pathological state arising from an abnormal immune response of the body to substances and tissues that are normally present in the body. In some embodiments, an autoimmune disease is any autoimmune disease known or described in the art. In some embodiments, an autoimmune disease is selected from: Addison's disease, arteriosclerosis, arteriosclerosis, atherosclerosis, atrophic gastritis, autoimmune hemolytic anemia, bullous pemphigoid, chronic active hepatitis, climacterium praecox, discoid lupus erythematosus, Goodpasture's syndrome, Hashimoto's thyroiditis, insulin resistant diabetes, juvenile diabetes, lens-induced uveitis, male infertility, mixed connective tissue disease, multiple sclerosis, myasthenia gravis, paroxysmal hemoglobinuria, pemphigus vulgaris, pernicious anemia, polymyositis, primary biliary liver cirrhosis, primary myxedema, rapidly progressive glomerulonephritis, scleroderma, Sjogren syndrome, sudden thrombocytopenic purpura, sympathetic phlebitis, systemic lupus erythematosus, thyrotoxicosis, and ulcerative colitis.

cDNA: The terms “cDNA” or “complementary DNA”, as used herein, refers to DNA that is complementary to messenger RNA; e.g., a DNA synthesized from a mRNA using an enzyme with reverse transcriptase activity.

Chromosome: The term “chromosome”, as used herein, refers to a strand of DNA, generally double-stranded and linear in eukaryotes, and usually double-stranded and circular in prokaryotes and mitochondria, and usually double- or single-stranded and circular or linear in viruses, which is inherited by individual organisms from its parent(s) and which carries one or more genes and/or other sequences; humans have 22 pairs of chromosome plus two sex chromosomes. The term “chromosome”, as used herein, includes DNA found in the nucleus, the mitochrondria, or any other location within a cell. The term “chromosome”, as used herein, includes chromosomes from any organism, including a bacterium, virus, yeast and other fungus, plant, or any prokaryote or eukaryote, including any animal or mammal.

Chromosomal fragment: The terms “chromosomal fragment”, “chromosome fragment,” “fragment of a chromosome” and the like, as used herein, refer to nucleic acids which comprise a portion of the chromosome; e.g., a chromosomal fragment may be truncated on one or both ends, yielding a nucleic acid comprising a significant portion of a chromosome. In some embodiments, the method of phasing allelic variants of a first and a second genetic locus includes the step of amplification of portions of a chromosomal fragment, wherein the chromosomal fragment comprises both the first and second genetic loci.

Complementary, Complementarity and other terms: The terms “Complementary”, “substantially complementary” and related terms, as used herein, refer to ability of nucleic acids (or portions thereof) to hybridize or base pair in a sequence-dependent manner to form a duplex; this includes, as non-limiting examples, hybridization between the two strands of nucleic acids, between an oligonucleotide primer and a primer binding site on a nucleic acid (e.g., a nucleic acid template), or between regions of two different primers. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner (e.g., non-Watson-Crick base pairing) that allows for the formation of duplexes. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984). Perfect complementarity, full complementarity or 100% complementarity refers to the situation in which each nucleotide unit of one polynucleotide strand can hydrogen bond (e.g., form Watson-Crick basepairing) with a nucleotide unit of a second polynucleotide strand, without a “mismatch”. Less than perfect complementarity refers to the situation in which not all nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. Substantial complementarity refers to about 79%, about 80%, about 85%, about 90%, about 95%, or greater complementarity. Thus, for example, two polynucleotides of 29 nucleotide units each, wherein each comprises a di-dT at the 3′ terminus such that the duplex region spans 27 bases, and wherein 27 of the 27 bases of the duplex region on each strand are complementary, are substantially complementary. In determining complementarity, overhang regions are excluded. As used herein, the term “region of complementarity”, as used herein, refers to a nucleic acid or portion thereof (or the sequence thereof), which is complementary to another nucleic acid or portion thereof (or the sequence thereof). In some embodiments, the region of complementarity is GC-rich. As used herein, the term “GC-rich” means that a particular sequence has a plurality or majority of G or C nucleotides (G-C) (as opposed to A, T or U); in some embodiments, the GC-rich sequence is at least about 50, 52.5, 55, 57.5, 60, 62.5, 65, 67.5 or 70% G-C. In some embodiments, the region of complementarity is at least 5, 10, 15, 20, 25, 30, 35, 40, 45, 50 nt long. As a non-limiting example, two primers can comprise a region of complementarity, in that the primers comprise sequences which are complementary to each other.

Droplet: The term “droplet”, as used herein, refers to a small drop or small volume. In some embodiments, a droplet is a non-limiting example of an aliquot, reaction aliquot or composition in a collection of compositions. In some embodiments, a droplet is a droplet in an emulsion.

Gene: The term “gene”, as used herein, refers to a nucleic acid (or portion thereof), or the sequence thereof, which is the basic unit of heredity and which usually specifies the sequence of a protein or nucleic acid product and/or sequences involved in biological functions (such as regulatory sequences involved in the control of transcription, translation, DNA replication, etc.); in some embodiments, the term “gene” refers to a nucleic acid molecule (or portion thereof) comprising an open reading frame and including at least one exon and (optionally) an intron sequence; in at least some cases, a gene is capable of encoding a particular protein after being transcribed and translated. The term “intron” refers to a DNA sequence present in a given gene which is spliced out during mRNA maturation. In some embodiments, the term “gene”, as used herein, includes sequences which determine the sequence of RNAs, such as pre-miRNA, pri-miRNA, miRNA, lncRNA, snoRNA, piRNA, tRNA, mRNA, or any other RNA transcript; in some embodiments, the term “gene”, as used herein, comprises sequences, including but not limited to, regulatory sequences and binding sites for any RNA, protein or any other molecule which can bind to a nucleic acid, including, for example, any of: RNA binding site, protein binding site, miRNA binding site, promoter, operator, repressor, transcription enhancer, transcriptional stop signal, DNA replication origin, DNA replication origin enhancer, and binding site for RNA or any other factor involved in splicing, RNA transcription, translation, DNA replication, reverse transcription, or any other cellular process (and sequences determining those sequences, such as, as non-limiting example, DNA sequences determining a corresponding RNA sequence bound by a protein, another RNA or any of the molecule). In some embodiments of the present disclosure, a gene can comprise two or more genetic loci.

Genetic locus and related terms: The terms “genetic locus”, “sequence element” and related terms, as used herein, refers to a portion of a genome, chromosome or nucleic acid of interest. In some embodiments, genetic locus can be from a single nucleotide to a segment of dozens or hundreds of nt in length or more. In some embodiments, a genetic locus can be present in the form of an allele; thus, in some embodiments, two or more alleles or allelic variants can exist for a particular genetic locus. In some embodiments, the genetic locus is a polymorphic site or polymorphism, which is a localized region within a chromosome at which the nucleotide sequence varies from a reference sequence in at least one individual in a population. In various embodiments, sequence variations can be substitutions, insertions or deletions of one or more bases. In some embodiments, the genetic locus is selected from: a SNP, a RFLP, an AFLP, an isozyme, a SSR, a mutation, a genetic lesion, a SNP, a repeat expansion, a deletion, a truncation, an insertion, an inversion, fusion, or a region of chromosome breakage and/or chromosome breakage and/or re-attachment.

Genotype: The term “genotype”, as used herein, refers to the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

Haplotype: The term “haplotype”, as used herein, is a contraction of the phrase “haploid genotype”. In some embodiments, a haplotype is a set of nucleotide sequence polymorphisms, genetic loci, allelic variants, or alleles present on a single maternal or paternal chromosome, usually inherited as a unit. In some embodiments, a haplotype is a set of alleles of closely linked loci on a chromosome that are generally inherited together. For example, a polymorphic allele at a first site in a nucleic acid sequence on the chromosome may be found to be associated with another polymorphic allele at a second site on the same chromosome, at a frequency other than would be expected for a random associate (e.g. “linkage equilibrium”). These two polymorphic alleles may be described as being in “linkage disequilibrium.” A haplotype may comprise two, three, four, or more alleles. The set of alleles in a haplotype along a given segment of a chromosome are generally transmitted to progeny together unless there has been a recombination event. A “haplotype” is the genotype of an individual at a plurality of genetic loci, i.e. a combination of alleles. Typically, the genetic loci described by a haplotype are physically and genetically linked, i.e., on the same chromosome segment. In some embodiments, haplotype information refers to information related to the phasing of allelic variants on various chromosomes (e.g., whether particular allelic variants are on the same or different chromosomes).

Homozygous: The term “homozygous”, as used herein, refers to having the same alleles at a one or more gene loci on homologous chromosome segments, or having identical pairs of genes for any given pair of hereditary characteristics. In some embodiments, an organism is homozygous for a particular gene or genetic locus if the sequence of that gene or genetic locus on one chromosome is the same as the corresponding sequence on another chromosome.

Heterozygous, hemizygous and nullizygous: The term “heterozygous”, as used herein, refers to the condition of having dissimilar pairs of a genetic locus or gene for any hereditary characteristic; in some embodiments, an organism is heterozygous for a particular gene or genetic locus if the sequence of that gene or genetic locus on one chromosome is different than the corresponding sequence on another chromosome. In some embodiments, a diploid organism is heterozygous at a genetic locus when its cells contain two different alleles of a gene; the cell or organism is called a heterozygote specifically for the allele of the genetic locus in question, therefore, heterozygosity refers to a specific genotype. In some embodiments, heterozygous genotypes are represented by a capital letter (representing the dominant allele) and a lowercase letter (representing the recessive allele), such as “Rr” or “Ss”. Alternatively, a heterozygote for gene “R” is assumed to be “Rr”. In some embodiments, the capital letter is written first. In some embodiments, if the trait in question is determined by simple (complete) dominance, a heterozygote will express only the trait coded by the dominant allele, and the trait coded by the recessive allele will not be present. In more complex dominance schemes the results of heterozygosity can be more complex. The term “compound heterozygous”, as used herein, refers the condition of having dissimilar pairs of two or more genes or genetic loci; in some embodiments, an organism is compound heterozygous for two or more genes or genetic loci if the sequences for each of the genes or genetic loci on one chromosome are different from the corresponding sequences for each of the genes or genetic loci on another chromosome. In some embodiments, an organism is a compound heterozygote when it has two recessive alleles for the same gene, but with those two alleles being different from each other (for example, both alleles might be mutated but at different locations). Compound heterozygosis reflects the diversity of the mutation base for many autosomal recessive genetic disorders; mutations in most disease-causing genes have arisen many times. This means that many cases of disease arise in individuals who have two unrelated alleles, who technically are heterozygotes, but both the alleles are defective. The term “hemizygous”, as used herein, refers to the condition of having only one of a pair of a genetic locus or gene for any hereditary characteristic; in some embodiments, an organism is hemizygous for a particular gene or genetic locus if the sequence of that gene or genetic locus on one chromosome is present and the corresponding sequence on another chromosome is missing (e.g., via a deletion or truncation). For organisms in which the male is heterogametic, such as humans, almost all X-linked genes are hemizygous in males with normal chromosomes because they have only one X chromosome and few of the same genes are on the Y chromosome. In some embodiments, transgenic mice generated through exogenous DNA microinjection of an embryo's pronucleus are also considered to be hemizygous because the introduced allele is expected to be incorporated into only one copy of any locus. A transgenic can later be bred to homozygosity and maintained as an inbred line to reduce the need to confirm the genotypes of each litter. In cultured mammalian cells, such as the Chinese hamster ovary cell line, a number of genetic loci are present in a functional hemizygous state, due to mutations or deletions in the other alleles. The term “nullizygous”, as used herein, refers to the condition of having two mutant or non-function members of a pair of a genetic locus or gene for any hereditary characteristic; in some embodiments, an organism is nullizygous for a particular gene or genetic locus if the sequence of that gene or genetic locus on each of the two chromosomes is mutant (e.g., null or non-functional). A nullizygous organism carries two mutant alleles for the same gene.

Hybridization: The term “hybridization”, as used herein, means the annealing of complementary nucleic acid molecules. In some embodiments, the term “hybridization” means one or more processes for co-localizing complementary, single-stranded nucleic acids, and/or co-localizing complementary non-traditional molecules (e.g., a polymer comprising modified nucleotides and/or nucleotide analogs) with single- or double-stranded nucleic acids through strand separation (e.g., by denaturation) and re-annealing, for example. In some embodiments, complementary nucleic acid molecules, optionally oligonucleotides, may hybridize to single- or double-stranded DNA. Methods for hybridization are known in the art, and include, but are not limited to, conditions for low and high stringency hybridization (Sambrook and Russell. (2001) Molecular Cloning: A Laboratory Manual 3rd edition. Cold Spring Harbor Laboratory Press; Sambrook, Fritsch, Maniatis. Molecular Cloning: A Laboratory Manual 3rd edition). Stringency of the hybridization may be controlled (e.g., by the washing conditions) to require up to 100% complementarity between the probe and the target sequence (high stringency), or to allow some mismatches between the probe and the target sequence (low stringency). Example factors to determine the appropriate hybridization and wash conditions based on the target and the probe are known in the art.

Linkage equilibrium and disequilibrium: The term “linkage disequilibrium”, “LD”, as used herein, refers to a non-random segregation of genetic loci or traits (or both). In any case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.). As used herein, linkage can be between two markers, or alternatively between a marker and a phenotype. A marker locus can be “associated with” (linked to) a particular trait. The degree of linkage of a molecular marker to a phenotypic trait is measured, e.g., as a statistical probability of co-segregation of that molecular marker with the phenotype. Linkage disequilibrium is most commonly assessed using the measure r², which is calculated using the formula described by Hill, W. G. and Robertson, A, Theor. Appl. Genet. 38:226-231 (1968). When r²=1, complete LD exists between the two marker loci, meaning that the markers have not been separated by recombination and have the same allele frequency. In some embodiments, values for r² above 1/3 indicate sufficiently strong LD to be useful for mapping (Ardlie et al., Nature Reviews Genetics 3:299-309 (2002)). In some embodiments, alleles are in linkage disequilibrium when r² values between pairwise marker loci are greater than or equal to 0.33, 0.4, 0.5, 0.6, 0.7, 0.8, 0.9, or 1.0. As used herein, “linkage equilibrium” describes a situation where two markers independently segregate, e.g., sort among progeny randomly. Markers that show linkage equilibrium are considered unlinked (whether or not they lie on the same chromosome). In some embodiments “linked loci” are located in close proximity such that meiotic recombination between homologous chromosome pairs does not occur with high frequency (frequency of equal to or less than 10%) between the two loci, e.g., linked loci co-segregate at least about 90% of the time, e.g., 91%, 92%, 93%, 94%, 95%, 96%, 97%, 98%, 99%, 99.5%, 99.75%, or more of the time. Marker loci are especially useful when they demonstrate a significant probability of co-segregation (linkage) with a desired trait (e.g., increased head smut resistance). For example, in some aspects, these markers can be termed “linked QTL markers”.

Mismatch: The terms “Mismatch” or “non-complementary nucleobase” and the like, as used herein, refer to the case when a nucleobase of a first nucleic acid is not capable of pairing with the corresponding nucleobase of a second or target nucleic acid.

mRNA: The terms “mRNA” or messenger RNA”, as used herein, refer to RNA, often synthesized from a DNA template via transcription, that mediates the transfer of genetic information from the cell nucleus to ribosomes in the cytoplasm, where the mRNA serves as a template for protein synthesis. In some embodiments, mRNA is processed, edited and/or transported within a cell. In some embodiments, a mRNA includes a precursor mRNA or a mature mRNA, or a processed, unprocessed or partially processed RNA. In some embodiments, a mRNA comprises a 5′ cap and/or a poly(A) tail. In some embodiments, a mRNA comprises a coding segment. In some embodiments, a mRNA does not comprise a coding segment. In some embodiments, a mRNA or fragment thereof comprises an exon and/or an intron. In some embodiments, a mRNA or fragment thereof comprises an exon. In a non-limiting example, a mRNA or fragment thereof comprising two or more genetic loci can act as a nucleic acid template, which can be used to generate a nucleic acid polymer, which comprises the two or more genetic loci and which can be phased, thereby indicating the phasing of the two or more genetic loci on the mRNA and thus the chromosome from which it was transcribed. In some embodiments, two or more genetic loci which are to be phased are all comprised in one or more exons. In some embodiments, one or more of two or more genetic loci which are to be phased are comprised in one or more introns. In some embodiments, one or more of two or more genetic loci which are to be phased are comprised in one or more introns and comprised in one or more exons.

Nucleic acid: The term “nucleic acid”, as used herein, includes any monomer, dimer, trimer, tetramer or polymer comprising nucleotides, modified nucleotides and/or nucleotide analogs. The term “polynucleotide” as used herein refer to a polymeric form of any length of nucleotides, modified nucleotides and/or nucleotide analogs, including ribonucleotides (RNA) or deoxyribonucleotides (DNA). These terms include the primary structure of the molecules and, thus, include double- and single-stranded DNA, and double- and single-stranded RNA. In some embodiments, these terms include analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges or internucleotidic linkage. The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. The prefix poly-refers to a nucleic acid, in some embodiments, containing 2 to about 10,000 nucleotide monomer units and wherein the prefix oligo-refers to a nucleic acid containing, in some embodiments, 2 to about 200 nucleotide monomer units. In some embodiments, a nucleic acid includes, but not limited to, deoxyribonucleotides or ribonucleotides and polymers thereof, for example, in at least partially single- or double-stranded form. In some embodiments, a nucleic acid includes any nucleotides, modified nucleotides, and/or nucleotide analogs, and polymers thereof. In some embodiments, a polynucleotide includes a polymeric form of nucleotides of any length, either ribonucleotides (RNA) or deoxyribonucleotides (DNA). Analogs of RNA and DNA (e.g., nucleotide analogs) include, but are not limited to: Morpholino, PNA, LNA, BNA, TNA, GNA, ANA, FANA, CeNa, HNA, cEt, tc-DNA, XNA, and UNA. Modified nucleotides include those which are modified in the phosphate, sugar, and/or base. Such modifications include sugar modifications at the 2′ carbon, such as 2′-MOE, 2′-OMe, and 2′-F. In some embodiments, a nucleic acid includes a poly- or oligo-ribonucleotide (RNA) and poly- or oligo-deoxyribonucleotide (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, a nucleic acid is an oligonucleotide, an antisense oligonucleotide, an RNAi agent, a miRNA, splice switching oligonucleotide (SSO), immunomodulatory nucleic acid, an aptamer, a ribozyme, a Piwi-interacting RNA (piRNA), a small nucleolar RNA (snoRNA), a mRNA, a lncRNA, a ncRNA, an antigomir (e.g., an antagonist to a miRNA, lncRNA, ncRNA or other nucleic acid), a plasmid, a vector, or a portion thereof. In some embodiments, a nucleic acid composition is a chirally controlled nucleic acid composition. In some embodiments, a nucleic acid composition is a chirally controlled oligonucleotide composition, or a chirally controlled nucleic acid composition. In some embodiments, a base includes a part (or a modified variant thereof) of a nucleic acid that is involved in the hydrogen-bonding that binds one nucleic acid strand to another complementary strand in a sequence-specific manner. The naturally occurring bases, e.g., guanine (G), adenine (A), cytosine (C), thymine (T), uracil (U), etc., are derivatives of purine (Pu) or pyrimidine (Py), though it should be understood that both naturally and non-naturally occurring base analogs are included. In some embodiments, the nucleobases are modified adenine, guanine, uracil, cytosine, or thymine. In some embodiments, the nucleobases are optionally substituted adenine, guanine, uracil, cytosine, or thymine. In some embodiments, the modified nucleobase mimics the spatial arrangement, electronic properties, an/or some other physicochemical property of the nucleobase and retains the property of hydrogen-bonding that binds one nucleic acid strand to another in a sequence specific manner. In some embodiments, a modified nucleobase can pair with all of the five naturally occurring bases selected from uracil, thymine, adenine, cytosine, or guanine without substantially affecting the melting behavior, recognition by intracellular enzymes or activity of the oligonucleotide duplex. Various additional modifications of the bases are known in the art. In some cases, a nucleic acid sequence can be defined as a sequence of bases, generally presented in the 5′ to 3′ direction. In some embodiments, while in the context of a nucleic acid, a base is normally conjugated to a sugar which forms the backbone along with an internucleotidic linkage (e.g., a phosphate or phosphorothioate or other modified internucleotidic linkage), a base does not comprise a sugar or an internucleotidic linkage. In some embodiments, a nucleoside includes a unit consisting of: (a) a base covalently bound to (b) a sugar. The base and/or sugar can be modified or not modified. In some embodiments, a sugar, as referenced herein in the context of referencing a nucleic acid, includes a monosaccharide in closed and/or open form. Naturally occurring sugars include the pentose (five-carbon sugar) deoxyribose (which forms DNA) or ribose (which forms RNA), though it should be understood that both naturally and non-naturally occurring sugar analogs are included. Sugars include, but are not limited to, ribose, deoxyribose, pentofuranose, pentopyranose, and hexopyranose moieties. As used herein, the term also encompasses structural analogs used in lieu of conventional sugar molecules, such as glycol, polymer of which forms the backbone of the nucleic acid analog, glycol nucleic acid (“GNA”). A deoxynucleoside comprises a deoxyribose. In some cases, a nucleic acid sequence can be defined as a sequence of bases and sugar modifications. In some embodiments, a sugar includes a modified sugar or unmodified sugar. In some embodiments, a modified sugar includes, as referenced in the context of a nucleic acid, a sugar which has been modified or a moiety that can functionally replace a sugar in a nucleic acid or modified nucleic acid. The modified sugar mimics the spatial arrangement, electronic properties, and/or some other physicochemical property of a sugar. A modified sugar, as a non-limiting example, can have a modification at the 2′ carbon. Various modifications include 2′-MOE, 2′-OMe and 2′-F. Various additional modifications of the sugar are known in the art. In some embodiments, a nucleotide includes a monomeric unit of a polynucleotide that consists of a base, a sugar, and a phosphate internucleotidic linkage, each of which can be optionally and independently modified. In some embodiments, a nucleotide is a subunit of a polynucleotide, nucleic acid or oligonucleotide. Each base, sugar and phosphate internucleoside linker can be independently modified or not modified. Many internucleotidic linkages are known in the art (such as, though not limited to, phosphate, phosphorothioates, boranophosphates and the like). In some embodiments, a nucleic acid includes one or more modified internucleotidic linkages such as PNAs (peptide nucleic acids) linkages, phosphotriesters, phosphorothionates, H-phosphonates, phosphoramidates, boranophosphates, methylphosphonates, phosphonoacetates, thiophosphonoacetates, etc. In some embodiments, an internucleotidic linkage includes linkage between nucleoside units of an oligonucleotide. In some embodiments, such a linkage comprises a phosphorus atom. In some embodiments, the linkage is refered to as “p”. In some embodiments, an intemucleotidic linkage is a phosphodiester linkage, as found in naturally occurring DNA and RNA molecules. In some embodiments, a linkage is a phosphorothioate. In some embodiments, backbone of an oligonucleotide or a nucleic acid includes the alternating sugars and intemucleotidic linkages (e.g., a phosphodiester or phosphorothioate). Unless otherwise specified, the term encompasses nucleic acids containing known analogues of natural nucleotides which have similar binding properties as the reference nucleic acid and are metabolized in a manner similar to naturally occurring nucleotides. In some embodiments, a particular nucleic acid sequence also encompasses conservatively modified variants (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Also included are molecules having naturally occurring phosphodiester linkages as well as those having non-naturally occurring linkages, e.g., for stabilization purposes. A nucleic acid can be in any physical form, e.g., linear, circular, nicked, or supercoiled. In some embodiments, one or more nucleotides are modified and contain moieties of DNA, peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), constrained ethyl (cEt), tricyclo-DNA (tc-DNA), xeno nucleic acid (XNA), unlocked nucleic acid (UNA), etc. In some embodiments, a nucleic acid comprises a modified internucleoside linker. In some embodiments, non-limiting examples of a nucleic acid include a nucleic acid template, a nucleic acid polymer, primers, probes, etc.

Nucleotide: The term “nucleotide” as used herein refers to a monomeric unit of a polynucleotide that consists of a heterocyclic base, a sugar, and one or more phosphate groups or phosphorus-containing internucleotidic linkages. The naturally occurring bases, (guanine, (G), adenine, (A), cytosine, (C), thymine, (T), and uracil (U)) are derivatives of purine or pyrimidine, though it should be understood that other naturally and non-naturally occurring base analogs are also included. The naturally occurring sugar include the pentose (five-carbon sugar) deoxyribose (which forms DNA) or ribose (which forms RNA), though it should be understood that other naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a modified nucleotide or nucleotide analog is any modified nucleotide or nucleotide analog described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 20070900071; WO 20070900071; or WO 2016/079181.

Nucleoside: The term “nucleoside”, as used herein, refers to a moiety wherein a nucleobase or a modified nucleobase is covalently bound to a sugar or modified sugar.

Sugar: The term “sugar”, as used herein, refers to a saccharide, in some embodiments, a monosaccharide in closed and/or open form. Sugars include, but are not limited to, ribose, deoxyribose, pentofuranose, pentopyranose, and hexopyranose moieties. As used herein, the term also encompasses structural analogs used in lieu of conventional sugar molecules, such as glycol, polymer of which forms the backbone of the nucleic acid analog, glycol nucleic acid (“GNA”).

Modified sugar: The term “modified sugar”, as used herein, refers to a moiety that can replace a sugar, in some embodiments, in nucleic acids. The modified sugar mimics the spatial arrangement, electronic properties, an/or some other physicochemical property of a sugar. In some embodiments, a modified sugar comprises a modification at a 2′ carbon. In some embodiments, a modified sugar comprises a 2′-F, 2′-OMe or 2′-MOE.

Nucleobase: The term “nucleobase”, as used herein, refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, the naturally-occurring nucleobases are modified adenine, guanine, uracil, cytosine, or thymine. In some embodiments, the naturally-occurring nucleobases are methylated adenine, guanine, uracil, cytosine, or thymine. In some embodiments, a nucleobase is a “modified nucleobase,” e.g., a nucleobase other than adenine (A), guanine (G), uracil (U), cytosine (C), and thymine (T). In some embodiments, the modified nucleobases are methylated adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex.

DNA and other terms: The terms “DNA”, “DNA molecule” and the like, as used herein, refer to a polymeric form of deoxyribonucleotides (adenine, guanine, thymine, or cytosine) in its either single stranded form or a double-stranded helix. In some embodiments, this term refers only to the primary and secondary structure of the molecule, and does not limit it to any particular tertiary forms. In some embodiments, this term includes double-stranded DNA found, inter alia, in linear DNA molecules (e.g., restriction fragments), viruses, plasmids, and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). In some embodiments, bases, sugars, and/or phosphate linkages of a DNA are independently and optionally modified.

Coding sequence: A DNA “coding sequence” or “coding region” is a double-stranded DNA sequence which is transcribed and translated into a polypeptide in vivo when placed under the control of appropriate expression control sequences. The boundaries of the coding sequence (the “open reading frame” or “ORF”) are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence is, usually, be located 3′ to the coding sequence. The term “non-coding sequence” or “non-coding region” refers to regions of a polynucleotide sequence that are not translated into amino acids (e.g. 5′ and 3′ un-translated regions).

Reading frame: The term “reading frame”, as used herein, refers to one of the six possible reading frames, three in each direction, of a double stranded DNA molecule. The reading frame that is used determines which codons are used to encode amino acids within the coding sequence of a DNA molecule.

Antisense: The term “antisense”, as used herein, for example, in reference to a nucleic acid, refers to a nucleic acid molecule which comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid molecule can associate via hydrogen bonds to a sense nucleic acid molecule. In some embodiments, an antisense oligonucleotide is capable of annealing to a target mRNA in a sequence-specific manner and mediating degradation of the mRNA via a RNaseH-dependent mechanism. In some embodiments, an antisense nucleic acid includes, as a non-limiting example, an antisense strand of a siRNA or other RNAi agent, which is capable of anneal to a target mRNA in a sequence-specific manner and mediating degradation of the mRNA via a RISC (RNA inhibition silencing complex)-mediated mechanism. In some embodiments, an antisense strand of a siRNA or other RNAi agent is annealed to a corresponding sense strand; in some embodiments, an antisense strand of a siRNA or other RNAi agent is not annealed to a corresponding sense strand.

Homology: The terms “Homology” or “identity” or “similarity”, as used herein, refers to sequence similarity between two nucleic acid molecules. Homology and identity can each be determined by comparing a position in each sequence which can be aligned for purposes of comparison. When an equivalent position in the compared sequences is occupied by the same base, then the molecules are identical at that position; when the equivalent site occupied by the same or a similar nucleic acid residue (e.g., similar in steric and/or electronic nature), then the molecules can be referred to as homologous (similar) at that position. Expression as a percentage of homology/similarity or identity refers to a function of the number of identical or similar nucleic acids at positions shared by the compared sequences. A sequence which is “unrelated” or “non-homologous” shares less than 40% identity, less than 35% identity, less than 30% identity, or less than 25% identity with a sequence described herein. In comparing two sequences, the absence of residues (amino acids or nucleic acids) or presence of extra residues also decreases the identity and homology/similarity. In some embodiments, the term “homology” describes a mathematically based comparison of sequence similarities which is used to identify genes with similar functions or motifs.

Identity: As used herein, “identity” means the percentage of identical nucleotide residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm can also be used to determine identity.

Heterologous: A “heterologous” region of a DNA sequence is an identifiable segment of DNA within a larger DNA sequence that is not found in association with the larger sequence in nature. Thus, when the heterologous region encodes a mammalian gene, the gene can usually be flanked by DNA that does not flank the mammalian genomic DNA in the genome of the source organism. Another example of a heterologous coding sequence is a sequence where the coding sequence itself is not found in nature (e.g., a cDNA where the genomic coding sequence contains introns or synthetic sequences having codons or motifs different than the unmodified gene). Allelic variations or naturally-occurring mutational events do not give rise to a heterologous region of DNA as defined herein.

Oligonucleotide: The term “oligonucleotide”, as used herein, refers to a polymer or oligomer of nucleotide monomers, containing any combination of nucleotides, modified nucleotides, nucleotide analogs, sugars, modified sugars, phosphate bridges, or modified phosphorus atom bridges (also referred to herein as “internucleotidic linkage”, defined further herein). Oligonucleotides can be single-stranded or double-stranded. As used herein, the term “oligonucleotide strand” encompasses a single-stranded oligonucleotide. A single-stranded oligonucleotide can have double-stranded regions and a double-stranded oligonucleotide can have single-stranded regions. Example oligonucleotides include, but are not limited to structural genes, genes including control and termination regions, self-replicating systems such as viral or plasmid DNA, single-stranded and double-stranded siRNAs and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides. Double-stranded and single-stranded oligonucleotides that are effective in inducing RNA interference are also referred to as siRNA, RNAi agent, or iRNA agent, herein. In some embodiments, these RNA interference inducing oligonucleotides associate with a cytoplasmic multi-protein complex known as RNAi-induced silencing complex (RISC). In many embodiments, single-stranded and double-stranded RNAi agents are sufficiently long that they can be cleaved by an endogenous molecule, e.g., by Dicer, to produce smaller oligonucleotides that can enter the RISC machinery and participate in RISC mediated cleavage of a target sequence, e.g. a target mRNA. Oligonucleotides of the present disclosure can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length. In some embodiments, a sequence of a nucleic acid or an oligonucleotide comprises or consists of a common base sequence hybridizes with a transcript of dystrophin, myostatin, Huntingtin, a myostatin receptor, ActRIIB, ActRIIA, SMN2, dystrophia myotonica protein kinase (DMPK), C9orf72, ApoE4, ApoC3, Proprotein convertase subtilisin/kexin type 9 (PCSK9), SMAD7 or KRT14 (Keratin 14). In some embodiments, a sequence of a nucleic acid or an oligonucleotide comprises or consists of a common base sequence hybridizes with a transcript of a gene related to Huntington's disease, spinal muscular atrophy, spinal muscular atrophy type 1, amyotrophic lateral sclerosis, Duchenne muscular dystrophy, myotonic dystrophy, myotonic dystrophy type 1, a genetic disease of the liver, a metabolic disease of the liver, epidermolysis bullosa simplex, a genetic disease of the skin, a genetic disease of the skin, or irritable bowel syndrome, or a genetic disease, or a metabolic disease.

Internucleotidic linkage: As used herein, the phrase “internucleotidic linkage”, “internucleotidic linker” and the like refer generally to a linkage, including but not limited to a phosphorus-containing linkage, between nucleotide units of an oligonucleotide, and is interchangeable with “inter-sugar linkage” and “phosphorus atom bridge,” as used above and herein. In some embodiments, an internucleotidic linkage is a phosphodiester linkage, as found in naturally occurring DNA and RNA molecules. In some embodiments, a modified internucleotidic linkage is an internucleotidic linkage which is not phosphorodiester. In some embodiments, an internucleotidic linkage is a “modified internucleotidic linkage”, wherein the internucleotidic linkage is not phosphodiester. In some embodiments of a modified internucleotidic linkage, each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described below. In some embodiments, a modified internucleotidic linkage is a phosphotriester linkage, phosphorothioate diester linkage

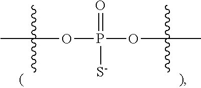 [SMILES: *OP(=O)([S-])O*]

or modified phosphorothioate triester linkage. It is understood by a person of ordinary skill in the art that the internucleotidic linkage can exist as an anion or cation at a given pH due to the existence of acid or base moieties in the linkage. In some embodiments, an example modified internucleotidic inkage is:

| Sym- | |
|---|---|
| bol | Modified Internucleotidic Linkage |
| s | 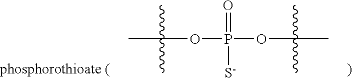 [SMILES: *OP(=O)([S-])O*] |
| s1 | 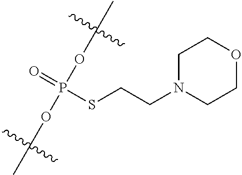 [SMILES: *OP(=O)(O*)SCCN1CCOCC1] |
| s2 | 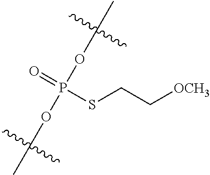 [SMILES: *OP(=O)(O*)SCCOC] |
| s3 | 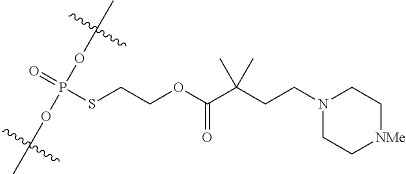 [SMILES: *OP(=O)(O*)SCCOC(=O)C(C)(C)CCN1CCN(C)CC1] |
| s4 | 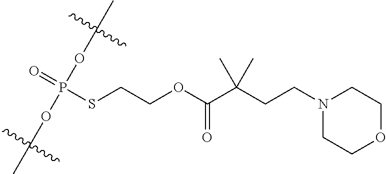 [SMILES: *OP(=O)(O*)SCCOC(=O)C(C)(C)CCN1CCOCC1] |
| s5 | 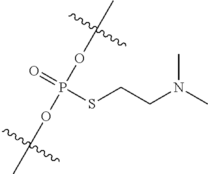 [SMILES: *OP(=O)(O*)SCCN(C)C] |
| s6 | 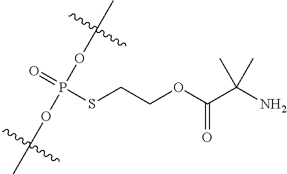 [SMILES: *OP(=O)(O*)SCCOC(=O)C(C)(C)N] |
| s7 | 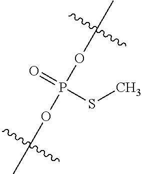 [SMILES: *OP(=O)(O*)SC] |
| s8 | 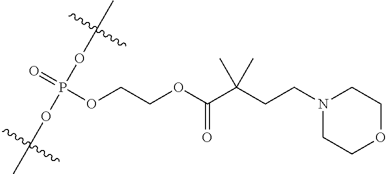 [SMILES: *OP(=O)(O*)OCCOC(=O)C(C)(C)CCN1CCOCC1] |
| s9 | 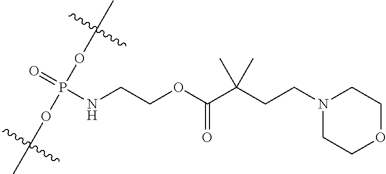 [SMILES: *OP(=O)(NCCOC(=O)C(C)(C)CCN1CCOCC1)O*] |
| s10 | 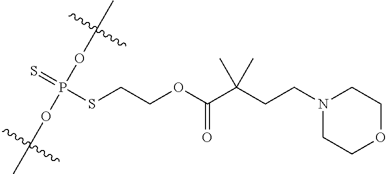 [SMILES: *OP(=S)(O*)SCCOC(=O)C(C)(C)CCN1CCOCC1] |
| s11 | 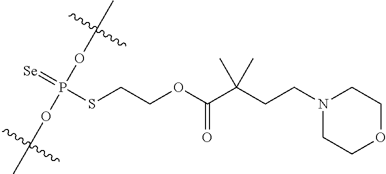 [SMILES: *OP(=[Se])(O*)SCCOC(=O)C(C)(C)CCN1CCOCC1] |
| s12 | 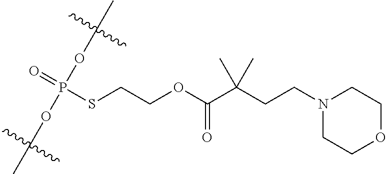 [SMILES: *OP(=O)(O*)SCCOC(=O)C(C)(C)CCN1CCOCC1] |
| s13 | 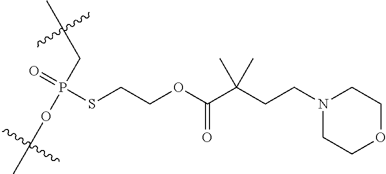 [SMILES: *CP(=O)(O*)SCCOC(=O)C(C)(C)CCN1CCOCC1] |
| s14 | 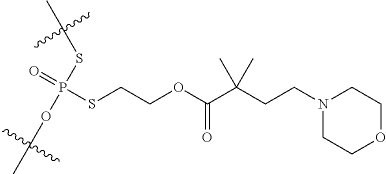 [SMILES: *OP(=O)(S*)SCCOC(=O)C(C)(C)CCN1CCOCC1] |
| s15 | 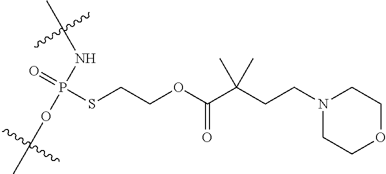 [SMILES: *NP(=O)(O*)SCCOC(=O)C(C)(C)CCN1CCOCC1] |
| s16 | 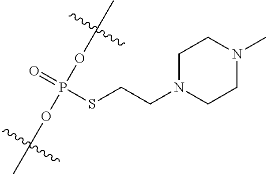 [SMILES: *OP(=O)(O*)SCCN1CCN(C)CC1] |
| s17 | 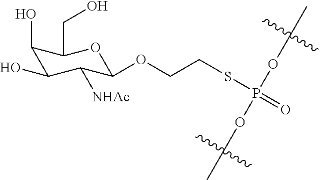 [SMILES: *OP(=O)(O*)SCCOC1OC(CO)C(O)C(O)C1NC(C)=O] |
| s18 | 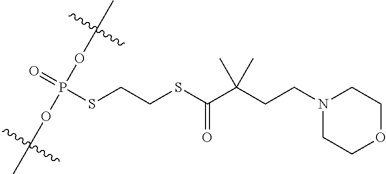 [SMILES: *OP(=O)(O*)SCCSC(=O)C(C)(C)CCN1CCOCC1] |

As a non-limiting example, (Rp, Sp)-ATsCs1GA has 1) a phosphorothioate internucleotidic linkage

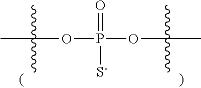 [SMILES: *OP(=O)([S-])O*]

between T and C; and 2) a phosphorothioate triester internucleotidic linkage having the structure of

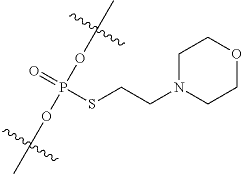 [SMILES: *OP(=O)(O*)SCCN1CCOCC1]

between C and G. Unless otherwise specified, the Rp/Sp designations preceding an oligonucleotide sequence describe the configurations of chiral linkage phosphorus atoms in the internucleotidic linkages sequentially from 5′ to 3′ of the oligonucleotide sequence. For instance, in (Rp, Sp)-ATsCs1GA, the phosphorus in the “s” linkage between T and C has Rp configuration and the phosphorus in “s1” linkage between C and G has Sp configuration. In some embodiments, “All-(Rp)” or “All-(Sp)” is used to indicate that all chiral linkage phosphorus atoms in oligonucleotide have the same Rp or Sp configuration, respectively. For instance, All-(Rp)-GsCsCsTsCsAsGsTsCsTsGsCsTsTsCsGsCsAsCsC indicates that all the chiral linkage phosphorus atoms in the oligonucleotide have Rp configuration; All-(Sp)-GsCsCsTsCsAsGsTsCsTsGsCsTsTsCsGsCsAsCsC indicates that all the chiral linkage phosphorus atoms in the oligonucleotide have Sp configuration. In some embodiments, in a modified internucleotidic linkage, a non-bridging oxygen in a phosphodiester is replaced by sulfur. In some embodiments, a modified internucleotidic linkage is a phosphorothioate. In some embodiments, in a modified internucleotidic linkage, both non-bridging oxygens in a phosphodiester are replaced by sulfur. In some embodiments, a modified internucleotidic linkage is a phosphorodithioate. In some embodiments, in a modified internucleotidic linkage, a bridging oxygen of the phosphodiester is replaced by sulfur. In some embodiments, a modified internucleotidic linkage is a phosphorothioic ether. In some embodiments, in a modified internucleotidic linkage, both bridging oxygens of the phosphodiester are replaced by sulfur. In some embodiments, in a modified internucleotidic linkage, a non-bridging oxygen in the phosphodiester is replaced by carbon. In some embodiments, in a modified internucleotidic linkage, any one or more oxygen is replaced by another atom which is not oxygen. In some embodiments, in a modified internucleotidic linkage, the phosphorus is replaced by another atom which is not phosphorus. In some embodiments, in a modified internucleotidic linkage, any one or more oxygens and the phosphorus are replaced by atoms which are not oxygen or phosphorus, respectively.

Linkage phosphorus: As defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester of an internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral.

Nucleic acid template: The term “nucleic acid template”, as used herein, refers to a nucleic acid, a portion or portions of which are copied or amplified to produce a nucleic acid polymer. In some embodiments, allelic variants of two or more genetic loci on the polymer can be phased in order to determine the arrangement of the allelic variants of the two or more genetic loci on a chromosome, mRNA or other genetic material from the source of the nucleic acid template. In some embodiments, a nucleic acid template is a chromosome, chromosomal fragment, genomic DNA, mRNA or cDNA.

Phasing: The term “phasing”, “to phase” and the like, as used herein, refer to the process or method of determining the linkage or phase of variants of genes or genetic loci on chromosomes. In some embodiments, phasing encompasses the method or process of determining the linkage, arrangement, and/or genetic sequence of alleles of two or more genetic loci on different chromosomes. In some embodiments, phasing relates to a relative position of different alleles or allelic variants on various chromosomes. In some embodiments, phasing can determine if two or more alleles or allelic variants are phased (or in phase) or unphased (or out of phase). The terms “phased” or “in phase”, as used herein, refers to particular alleles or allelic variants of two or more (e.g., multiple) genetic loci which are located on the same nucleic acid template (e.g., the same chromosome). The terms “unphased” or “out of phase”, as used herein, refers to particular alleles or allelic variants of two or more (e.g., multiple) genetic loci which are not located on the same nucleic acid template (e.g., the same chromosome). For example, at each genetic locus or polymorphic site, the sequence identities may be known for both copies of the locus of an individual, or multiple copies of a population but it is not known whether they are derived from the same allele of the chromosome.

Polymorphism: The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic locus can be a single nucleotide, the identity of which differs in the other alleles. A polymorphic locus can also be more than one nucleotide long. The allelic form occurring most frequently in a selected population is often referred to as the reference and/or wild-type form. Other allelic forms are typically designated alternative or variant alleles. Diploid organisms may be homozygous or heterozygous for allelic forms. A diallelic or biallelic polymorphism has two forms. A trialleleic polymorphism has three forms.

Polymorphic gene: The term “polymorphic gene”, as used herein, refers to a gene having at least one polymorphic region.

Repeat expansion: The term “repeat expansion”, as used herein, refers to a region of a nucleic acid wherein a short sequence (as non-limiting examples, a trinucleotide, tetranucleotide or hexanucleotide) is repeated again and again. In some embodiments, the excessive number of repeats is in the coding segment of a gene. In some embodiments, an excessive number of repeats is associated with a particular disorder. In some embodiments, the repeat expansion is an expansion of a trinucleotide, tetranucleotide, or hexanucleotide repeat. In some embodiments, the repeat expansion is associated with a disorder selected from: neurological disorder, Huntington's disease, fragile X syndrome, fragile X-E syndrome, fragile X-associated tremor/ataxia syndrome, dystrophy, myotonic dystrophy, juvenile myoclonic epilepsy, ataxia, Friedreich's ataxia, spinocerebellar ataxia, atrophy, spino-bulbar muscular atrophy, Dentatorubropallidoluysian atrophy, ALS, frontotemporal lobar degeneration, frontotemporal dementia, and asthma. The terms “repeat disorder”, “repeat expansion disorder” and the like, as used herein, refer to a pathological state which is associated with a repeat expansion, in which the number of adjacent trinucleotide repeats exceeds a number which is considered within the normal range, or below which is considered not to be associated with a particular disease. In some embodiments, a trinucleotide repeat disorder is a genetic disorder caused and/or associated with a trinucleotide repeat expansion, in which the number of adjacent trinucleotide repeats exceeds a number which is considered within the normal range, or below which is considered not to be associated with a particular disease.

Residue: The term “residue”, as used herein, refers to a subunit (e.g., a mer) in an oligomeric or polymeric molecule. In some embodiments, a residue is a nucleotide (nt) in a polynucleotide. In some embodiments, a residue is an amino acid residue in a peptide, oligopeptide or protein.

Sample: A “sample” as used herein is a specific organism or material obtained therefrom. In some embodiments, a sample is a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, a sample is an organism. In some embodiments, a sample is a plant. In some embodiments, a sample is an animal. In some embodiments, a sample is a human. In some embodiments, a sample is an organism other than a human.

SNP: The terms “SNP” or “single nucleotide polymorphism”, as used herein, refers to a variation in a single nucleotide that occurs at a specific position in the genome, where each variation is present to some appreciable degree within a population (e.g., >1%). In some embodiments, the terms “single nucleotide polymorphism” and “SNP”, as used herein, refer to a single nucleotide variation among genomes of individuals of the same species. For example, at a specific base position in the human genome, the base C may appear in most individuals, but in a minority of individuals, the position is occupied by base A. There is an SNP at this specific base position, and the two possible nucleotide variations—C or A—are said to be alleles for this base position. In some embodiments, there are only two different alleles. In some embodiments, a SNP is triallelic in which three different base variations may coexist within a population. Hodgkinson et al. 2009 Genetics 1. doi:10.4172/2157-7145.1000107. In some embodiments, SNPs underlie differences in individual-to-individual susceptibility to diseases; a wide range of human diseases, e.g. sickle-cell anemia, β-thalassemia and cystic fibrosis, etc. result from SNPs. Ingram 1956 Nature 178: 792-794; Chang et al. 1979 Proc. Natl. Acad. Sci. USA 76: 2886-2889; Hamosh et al. 1992 Am. J. Human Genet. 51: 245-250. In some embodiments, severity of illness and way a human body responds to treatments are also manifestations of genetic variations. For example, a single base mutation in the APOE (apolipoprotein E) gene is associated with a higher risk for Alzheimer's disease. Wolf et al. 2012 Neurobiology of Aging 34: 1007-17. In some embodiments, a particular SNP is not associated with a disease. In some embodiments, a SNP may be a single nucleotide deletion or insertion. In general, SNPs may occur relatively frequently in genomes and contribute to genetic diversity. In some embodiments, SNPs are mutationally more stable than other polymorphisms, lending their use in association studies in which linkage disequilibrium between markers and an unknown variant is used to map disease-causing mutations. The location of a SNP is generally flanked by highly conserved sequences. An individual may be homozygous or heterozygous for an allele at each SNP site. A heterozygous SNP allele can be a differentiating polymorphism. A SNP may be targeted with an antisense oligonucleotide. In some embodiments, a SNP is a polymorphic site at which the sequence variation is caused by substitution of a single base at a specific position. SNPs refer to nucleotide variations at defined genomic positions among a population. A SNP within a coding region, in which both forms lead to the same protein sequence, is termed synonymous; if different proteins are produced they are non-synonymous. In some embodiments, SNPs may have consequences for gene splicing, transcription factor binding, and/or the sequence of non-coding RNA, for example, and/or may indicate the haplotype of the organism. Large collections of confirmed and annotated SNPs are publicly available (e.g., The SNP Consortium, National Center for Biotechnology Information, Cold Spring Harbor Laboratory) [Sachidanandam et al. 2001 Nature 409: 928-933; The 1000 Genomes Project Consortium 2010 Nature 467: 1061-73 and Corrigendum; Kay et al. 2015 Mol. Ther. 23: 1759-1771].

In some embodiments, a “single nucleotide polymorphism site” or “SNP site” refers to the nucleotides surrounding a SNP contained in a target nucleic acid to which an antisense compound is targeted.

Upstream and downstream: The terms “upstream” and “downstream”, as used herein, refers to, in describing nucleic acid molecule orientation and/or polymerization are used herein as understood by one of skill in the art. As such, “downstream” generally means proceeding in the 5′ to 3′ direction, and “upstream” generally means the converse.

Wild-type: As used herein, the term “wild-type” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms (e.g., alleles).

### 2. Detailed Description of Certain Embodiments

Humans, among other living things, are diploid and determining the linkage of alleles of genetic loci on the same or different chromosomes is desirable for many reasons. The sequences on corresponding chromosomes are known as haplotypes. The process of determining which alleles are on which chromsomes is known as phasing or haplotyping. Phasing data can be used, for instance, for population genetic analysis of admixture, migration, and selection, but also for study of allele-specific gene regulation, compound heterozygosity, and their roles in human disease. Phasing information is useful in patient stratification, forensics and various other applications in the medical and bioscience fields. Twehey et al. 2011 Nat. Rev. Genet. 12: 215-223; and Glusman et al. 2014 Genome Med. 6:73.

Phasing data is important in some therapies, including allele-specific therapies. In some diseases, a genetic lesion such as a deleterious repeat, deletion, insertion, inversion or other mutation has been identified. In some patients, one allele of a gene can comprise a disease-associated mutation at a genetic locus, while the other allele is normal, wild-type or otherwise not disease-associated. In some embodiments, an allele-specific therapy can target an allele comprising a disease-associated mutation, but not the corresponding wild-type allele. In some embodiments, an allele-specific therapy can target an allele comprising a disease-associated mutation at a locus, but not by directly targeting the locus, but rather by targeting a different locus on the mutant allele. As a non-limiting example, an allele-specific therapy can target an allele comprising a disease-associated mutation at a locus by targeting a different locus in the same allele, such as a SNP (single nucleotide polymorphism) in the same gene.

As a non-limiting example, some disease-associated genetic lesions may be difficult to target or otherwise not readily amenable to targeting. As a non-limiting example, some genes comprise repeats (e.g., trinucleotide or tetranucleotide repeats); in some cases, a small number of repeats is not disease-associated, but an abnormally large number of repeats, or a repeat expansion, is disease-associated. Because the repeats exist on both the wild-type and mutant alleles, it may be difficult to target the disease-associated repeats directly. However, if a particular SNP variant exists on the allele with the disease-associated repeat expansion but not on the wild-type allele, that SNP variant can be used to target an allele-specific therapy which targets the mutant allele but not the wild-type allele.

As a non-limiting example, phasing data for an individual indicates if a particular SNP is in phase (e.g., on the same chromosome) as the lesion and thus that SNP can be targeted with a therapeutic nucleic acid. The therapeutic can then target the mutant gene, while not targeting the wild-type allele. Obtaining the phasing data to target only the mutant allele can be especially useful if expression of the wild-type allele is essential.

As another non-limiting example, phasing information is useful if it is known that an individual has both a wild-type and a mutant allele of each of two genetic loci on the same gene. Phasing information will reveal if both copies of the gene each have one mutant allele, or if one copy of the gene has two mutations, while the other is wild-type at both alleles. As another non-limiting example, phasing information may be useful in an analysis of a HLA (human leukocyte antigen) region, as particular genetic diseases have been associated with different haplotypes of the major histocompatibility complex.

In some embodiments, the present disclosure presents, inter alia, various methods for phasing genetic loci on a nucleic acid template. As non-limiting examples, the present disclosure presents methods for phasing a genetic locus such as a genetic lesion (such as an inversion, fusion, deletion, insertion or other mutation) and another genetic locus (such as a SNP) on a chromosome; the two genetic loci can be in the same gene, or in different genes.

### Phasing

In some embodiments the present disclosure provides methods for phasing, also known as haplotyping or haplotype phasing, that identifies which alleles are co-located on the same chromosome.

At a given gene locus on a pair of autosomal chromosomes, a diploid organism (e.g., a human being) inherits one allele of the gene from the mother and another allele of the gene from the father. At a heterozygous gene locus, two parents contribute different alleles (e.g., one A and one a). Without additional processing, it is impossible to tell which parent contributed which allele. Such genotype data that is not attributed to a particular parent is referred to as unphased genotype data. Typically, initial genotype readings obtained from genotyping chips are often in an unphased form.

Many sequencing procedures can reveal that an individual has sequence variability at particular positions. For example, at one position (e.g., a SNP), the individual may have a C in one copy of the gene and a G on the other. For a separate position (e.g., a different SNP), the individual may have a A in one copy and a U in the other. Because many sequencing techniques involve fragmentation of the nucleic acid template, depending on the sequencing technique used, it may not be possible to determine, for example, if the C and A or C and U are on the same chromosome. Phasing information will provide information on the arrangement of the different alleles on the different chromsomes.

As noted by Laver et al., phasing is also important in pharmacogenetics, transplant HLA typing and disease association mapping. Laver et al. 2016 Nature Scientific Reports 6:21746 DOI: 10.1038/srep21746. Phasing of allelic variants is important for clinical interpretation of the genome, population genetic analysis, and functional genomic analysis of allelic activity. The phasing of rare and de novo variants is crucial for identifying putative causal variants in clinical genetics applications, for example by distinguishing compound heterozygotes from two variants on the same allele.

For example, in some diseases, a subject can have two different alleles of the same gene, one of which is wild-type (not disease-associated) and the other is mutant (disease-associated). In some methods of disease treatment, a nucleic acid therapy (such as an antisense oligonucleotide or siRNA) targets the mutant allele, but not the wild-type allele, thus targeting the mutant but not the wild-type mRNA for degradation. In some of these therapies, however, the nucleic acid therapy may not directly target the mutant allele, but rather another genetic locus on the same genetic material. A nucleic acid therapy can be designed which targets the mRNA with the mutation, but does not directly target the site of the mutation. Instead, the nucleic acid therapy can target another genetic locus, such as a single nucleotide polymorphism or SNP, which is on the same mRNA as the mutant mRNA. Phasing is used in this example to determine if a particular SNP is on the same mRNA as the mutation and thus the SNP can be used to target the nucleic acid therapy.

Phasing can thus be used to, among other things, identify different alleles of various genetic loci, including but not limited to: a single nucleotide polymorphism (SNP), a restriction fragment length polymorphisms (RFLP), an amplified fragment length polymorphisms (AFLP), random amplified polymorphic DNA (RAPD), an isozyme, a simple sequence repeat (SSR), a mutation, a genetic lesion, a repeat expansion, a deletion, a truncation, an insertion, an inversion, fusion, or a region of chromosome breakage and/or chromosome breakage and/or re-attachment.

Phasing thus has many benefits and uses, including patient stratification for nucleic acid therapies.

### Limitations of Various Phasing Methods

Several techniques have been reported for phasing. In some embodiments the present disclosure encompasses recognition of liabilities and limitations of various reported techniques.

In some embodiments, the present disclosure appreciates that some sources of problems with prior technologies related to phasing include: the difficulty in phasing variant alleles of genetic loci which are very far apart from each on a chromosome; and the difficulty in phasing genetic loci which are intronic, etc.

As noted by Castel et al. [Castel et al. 2016 phASER: Long range phasing and haplotypic expression from RNA sequencing, doi: http://dx.doi.org/10.1101/039529], existing methods to phase variants are limited to phasing by transmission [Roach et al. 2011 Am. J. Hum. Genet. 89: 382-397], only available in familial studies, population based phasing [Delaneau et al. 2012 Nat. Methods 9: 179-181], which is ineffective for rare and de novo variants, phasing by sequencing long genomic fragments [Kuleshov et al. 2014 Nat. Biotech. 32: 261-266], which requires specialized and costly technology, phasing using expression data by inferring haplotype through allelic imbalance [Berger et al. 2015 Res. Comp. Mol. Biol. 9029: 28-29], which only applies to loci with well-detected allelic expression [Castel et al. 2015 Genome Biol. 16: 195] and physical techniques, for example those that employ allelic probes and microscopy, which are low throughput but high confidence [Regan et al. 2015 PloS ONE 10: e0118270]. Castel et al. also noted that more recently “read backed phasing” using readily available short read DNAsequencing (DNA-seq) has emerged [Yang et al. 2013 Bioinformatics 29: 2245-2252], however, it is limited by the relatively short distances which can be spanned by the reads.

In addition, Garg et al. stated that statistical or population-based phasing is less accurate for phasing rare variants and cannot be applied at all to private or de novo variants. Garg et al. 2016 Read-Based Phasing of Related Individuals. O'Connell also stated that the Lander-Green algorithm based approaches have computation and space complexity that scale exponentially with sample size; they can be sensitive to genotyping error and they can only phase sites where at least one member of the pedigree is not heterozygous. O'Connell et al. 2014 PLoS ONE 10: e1004234. Laver et al. stated that next generation sequencing technologies are often not able to phase variants that are more than a few hundred base pairs apart because of short read lengths. Laver et al. 2016 Nature Scientific Reports 6:21746 DOI: 10.1038/srep21746. Hickey et al. stated that long-range phasing (LRP) is not fully robust; applying LRP can result in parts of a given dataset being not phased or phased incorrectly. Hickey et al. 2011 Genet. Select. Evol. 43:12.

### Example Methods of Phasing

In some embodiments, the present disclosure provides methods for phasing allelic variants of genetic loci on a nucleic acid template, such as a chromosome, chromosomal fragment, genomic DNA, mRNA or cDNA. In some embodiments, the present disclosure provides compositions and methods pertaining to nucleic acids comprising two or more genetic loci. Provided nucleic acids are useful for many purposes, including, but not limited to, phasing.

In some embodiments, the present disclosure provided a method of phasing allelic variants of genetic loci on a nucleic acid template (e.g., a chromosome) in a sample, wherein the method comprises the step of partitioning the sample into multiple aliquots, which generally comprise no more than one copy of the template. A plurality of nucleic acid polymers is produced from the template; in some embodiments, in the process of generating the nucleic acid polymers, the spacing region between genetic loci is replaced or modified, in some embodiments, greatly shortening the spacing region. This generates a provided composition, which is easier to handle technically; determining the phasing of allelic variants of a nucleic acid polymer indicates the phasing of the original nucleic acid template.

In some embodiments, the present disclosure provides a method of phasing allelic variants of a first and a second genetic locus on a nucleic acid template comprising the first and the second genetic locus and a first spacing region between the first and the second genetic locus, the method comprising steps of:

(a) providing a sample comprising one or more types of the nucleic acid template;

(b) partitioning the sample into aliquots so that a plurality of the aliquots contain no more than one type of the template;

(c) generating, from at least one aliquot containing one type of the template, a plurality of nucleic acid polymers based on the template, wherein at least one of the nucleic acid polymers comprises the first and the second genetic locus and a second spacing region that is different from the first spacing region; and

(d) phasing the first and the second genetic locus on the at least one nucleic acid polymer to phase the allelic variants of the first and second genetic loci on the nucleic acid template.

In some embodiments, the present disclosure provides a method of phasing allelic variants of multiple genetic loci on a nucleic acid template comprising the multiple genetic loci and multiple spacing regions between the multiple genetic loci, the method comprising steps of:

(a) providing a sample comprising one or more types of the nucleic acid template;

(b) partitioning the sample into aliquots so that a plurality of the aliquots contain no more than one type of the template;

(c) generating, from at least one aliquot containing one type of the template, a plurality of nucleic acid polymers based on the template, wherein at least one of the nucleic acid polymers comprises the multiple genetic loci and wherein at least one of the multiple spacing regions on the nucleic acid polymer is different from the corresponding spacing region on the nucleic acid template; and

(d) phasing the multiple genetic loci on the at least one nucleic acid polymer to phase the allelic variants of the multiple genetic loci on the nucleic acid template.

In some embodiments, the present disclosure provides a method of phasing allelic variants of a first and a second genetic locus on a nucleic acid template comprising the first and the second genetic locus and a first spacing region between the first and the second genetic locus, the method comprising a step of:

(a) phasing the allelic variants on a nucleic acid polymer in order to phase the allelic variants on the nucleic acid template,

wherein the nucleic acid polymer comprises the first and the second genetic locus, and a second spacing region which is different from the first spacing region between the first and the second genetic locus,

wherein the nucleic acid polymer is generated from an aliquot containing one type of the nucleic acid template, and

wherein the aliquot is generated by partitioning a sample comprising one or more types of the nucleic acid template into aliquots so that a plurality of the aliquots contain no more than one type of the template.

In some embodiments, the present disclosure provides a method of phasing allelic variants of multiple genetic loci on a nucleic acid template comprising the multiple genetic loci and multiple spacing regions between the multiple genetic loci, the method comprising:

phasing the allelic variants on a nucleic acid polymer in order to phase the allelic variants on the nucleic acid template,

wherein the nucleic acid polymer comprises the multiple genetic loci, and wherein one or more of the multiple spacing regions on the nucleic acid polymer is different than one or more of the multiple spacing regions on the nucleic acid template,

wherein the nucleic acid polymer is generated from an aliquot containing one type of the nucleic acid template, and

wherein the aliquot is generated by partitioning a sample comprising one or more types of the nucleic acid template into aliquots so that a plurality of the aliquots contain no more than one type of the template.

In some embodiments, the present disclosure provides a method of generating nucleic acid polymers comprising a first and a second genetic locus, the method comprising steps of:

(a) providing a sample comprising one or more types of the nucleic acid template comprising the first and the second genetic locus and a first spacing region between the first and the second genetic locus;

(b) partitioning the sample into aliquots so that a plurality of the aliquots contain no more than one type of the template; and

(c) generating, from each aliquot containing one type of the template, a plurality of nucleic acid polymers based on the template, wherein at least one of the nucleic acid polymers comprises the first and the second genetic locus and a second spacing region that is different from the first spacing region.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

In some embodiments, the present disclosure provides a method of generating a plurality of nucleic acid polymers, wherein at least one of the polymers comprises a first and a second genetic locus of a nucleic acid template comprising the first and the second genetic locus and a first spacing region between the first and the second genetic locus, the method comprising a step of:

generating from a first aliquot of the nucleic acid template a plurality of nucleic acid polymers based on the template, wherein at least one of the nucleic acid polymers comprises the first and the second genetic locus and a second spacing region that is different from the first spacing region; and wherein:

the first aliquot contains no more than one type of the template; and

at least one second aliquot of the nucleic acid template comprises a different type of the nucleic acid template than the first aliquot.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

In some embodiments, the present disclosure provides a method comprising steps of:

(a) providing a collection of discrete reaction aliquots, at least one of which contains:

a single molecule of a nucleic acid template whose base sequence includes a first sequence element and a second sequence element, wherein the first and second sequence elements are separated from one another in the template nucleic acid by a spacing sequence element comprising a plurality of residues, and further wherein at least one of the first and/or second sequence elements is present in two or more different forms within the set of template nucleic acids present in the collection; and

reagents for amplifying nucleic acids;

(b) incubating the at least one reaction aliquot under conditions and for a time sufficient to generate, within the at least one reaction aliquot, a product nucleic acid in which the first and second sequences, or the complement sequences thereof, are linked to one another and some or all of the residues of the spacing element, or the complement thereof, are absent.

In some embodiments, the method further comprising the step of determining the forms of each of the first and second sequence element on the product nucleic acid, in order to determine the forms of each of the first and second sequence element on the nucleic acid template.

In some embodiments, the present disclosure provides a method comprising: incubating one or more discrete reaction aliquots of a collection, wherein each reaction aliquot of the collection contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection; and

reagents for amplifying nucleic acids;

under conditions and for a time sufficient to generate, within each discrete reaction aliquot, a product nucleic acid in which the first and second sequences, or the complement sequences thereof, are linked to one another and some or all of the residues of the spacing element, or the complement thereof, are absent.

In some embodiments, the method further comprises the step of determining the forms of each of the first and second sequence element on the product nucleic acid, in order to determine the forms of each of the first and second sequence element on the nucleic acid template.

In some embodiments, the present disclosure provides a composition comprising:

a single molecule of a nucleic acid template whose base sequence includes a first sequence element and a second sequence element, wherein the first and second sequence elements are separated from one another in the template nucleic acid by a spacing sequence element that comprises a plurality of residues;

a plurality of nucleic acid molecules in each of which the first and second sequence elements, or the complement sequences thereof, are linked to one another and some or all of the residues of the spacing sequence element, or the complement thereof, are absent; and

optionally an reagent for amplifying the template nucleic acid,

wherein no molecules other than the nucleic acid template and the plurality of nucleic acid molecules contain both the first and the second sequences.

In some embodiments, the present disclosure provides a composition comprising a collection of discrete compositions, each of which independently contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the first and/or second sequence elements is present in two or more different forms within the set of template nucleic acids present in the collection; and

a plurality of nucleic acid molecules, in each of which the first and second sequences, or the complement sequences thereof, are linked to one another and some or all of the residues of the spacing sequence element, or the complement thereof, are absent; and

optionally an reagent for amplifying the template nucleic acid.

In some embodiments, the present disclosure provides a composition comprising a collection of discrete compositions, each of which independently contains:

a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection;

a plurality of nucleic acid molecules in each of which the first and second sequences, or the complement sequences thereof, are linked to one another and some or all of the residues of the spacing element, or the complement thereof, are absent; and

optionally an reagent for amplifying the template nucleic acid;

wherein in each composition, no molecules other than the template nucleic acid and the plurality of nucleic acid molecules contain both the first and the second sequences.

In some embodiments, the first and/or second genetic locus or sequence element are selected from: a single nucleotide polymorphism (SNP), a restriction fragment length polymorphisms (RFLP), an amplified fragment length polymorphisms (AFLP), random amplified polymorphic DNA (RAPD), an isozyme, a simple sequence repeat (SSR), a mutation, a genetic lesion, a repeat expansion, a deletion, a truncation, an insertion, an inversion, fusion, or a region of chromosome breakage and/or chromosome breakage and/or re-attachment.

In some embodiments, the repeat expansion is an expansion of a trinucleotide, tetranucleotide, or hexanucleotide repeat.

In some embodiments, the repeat expansion is associated with a disorder.

In some embodiments, the repeat expansion is associated with a disorder selected from: neurological disorder, Huntington's disease, fragile X syndrome, fragile X-E syndrome, fragile X-associated tremor/ataxia syndrome, dystrophy, muscular dystrophy, myotonic dystrophy, juvenile myoclonic epilepsy, ataxia, Friedreich's ataxia, spinocerebellar ataxia, atrophy, spino-bulbar muscular atrophy, Dentatorubropallidoluysian atrophy, ALS, frontotemporal lobar degeneration, frontotemporal dementia, and asthma.

In some embodiments, the sample is from a patient afflicted with, showing symptoms of, suspected to have, having or suspected of having a genetic predisposition to, or susceptible to a disorder selected from cancer, autoimmune disease, infection, neurological, neuromuscular or neurodegenerative disease, and the first and/or second genetic locus is associated with the disorder.

In some embodiments, the first and second genetic loci or sequence element are on the same gene.

In some embodiments, the first and second genetic loci or sequence element are on different genes.

In some embodiments, the first spacing region is longer than the second spacing region.

In some embodiments, the length of the first spacing region at least 0.01, 0.025, 0.05, 0.075, 0.1, 0.25, 0.5, 0.75, 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, 1000 kb.

In some embodiments, the second spacing region is no more than 10, 25, 50, 100, 150, 200, 225, 250, 300, 350, 400, 450, 500 bp.

In some embodiments, the length of the second spacing region is at least 0.01, 0.025, 0.05, 0.075, 0.1, 0.25, 0.5, 0.75, 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, or 1000 kb shorter than the first spacing region. In some embodiments, the second spacing region is at least 0.01 kb shorter than the first spacing region. In some embodiments, the second spacing region is at least 0.025 kb shorter than the first spacing region. In some embodiments, the second spacing region is at least 0.05 kb shorter than the first spacing region. In some embodiments, the second spacing region is at least 0.075 kb shorter than the first spacing region. In some embodiments, the second spacing region is at least 0.1 kb shorter than the first spacing region. In some embodiments, the second spacing region is at least 0.25 kb shorter than the first spacing region. In some embodiments, the second spacing region is at least 0.5 kb shorter than the first spacing region. In some embodiments, the second spacing region is at least 0.75 kb shorter than the first spacing region. In some embodiments, the second spacing region is at least 1 kb shorter than the first spacing region. In some embodiments, the second spacing region is at least 2 kb shorter than the first spacing region. In some embodiments, the second spacing region is at least 5 kb shorter than the first spacing region. In some embodiments, the second spacing region is at least 5 kb shorter than the first spacing region. In some embodiments, the second spacing region is at least 10 kb shorter than the first spacing region. In some embodiments, the second spacing region is at least 20 kb shorter than the first spacing region. In some embodiments, the second spacing region is at least 30 kb shorter than the first spacing region. In some embodiments, the second spacing region is at least 40 kb shorter than the first spacing region. In some embodiments, the second spacing region is at least 50 kb shorter than the first spacing region. In some embodiments, the second spacing region is at least 60 kb shorter than the first spacing region. In some embodiments, the second spacing region is at least 70 kb shorter than the first spacing region. In some embodiments, the second spacing region is at least 80 kb shorter than the first spacing region. In some embodiments, the second spacing region is at least 90 kb shorter than the first spacing region. In some embodiments, the second spacing region is at least 100 kb shorter than the first spacing region. In some embodiments, the second spacing region is at least 125 kb shorter than the first spacing region. In some embodiments, the second spacing region is at least 150 kb shorter than the first spacing region. In some embodiments, the second spacing region is at least 175 kb shorter than the first spacing region. In some embodiments, the second spacing region is at least 200 kb shorter than the first spacing region. In some embodiments, the second spacing region is at least 250 kb shorter than the first spacing region. In some embodiments, the second spacing region is at least 300 kb shorter than the first spacing region. In some embodiments, the second spacing region is at least 400 kb shorter than the first spacing region. In some embodiments, the second spacing region is at least 500 kb shorter than the first spacing region. As demonstrated, in some embodiments, provided technologies are particularly useful when the first genetic locus and the second genetic locus are far apart.

In some embodiments, the nucleic acid template is selected from: a chromosome or fragment thereof, genomic DNA, mRNA and cDNA.

In some embodiments, the sample is selected from: tissue, cells, blood, sputum, cheek swab, urine, a Formalin-fixed, paraffin-embedded tissue sample (FFPE), and a prepared sample.

In some embodiments, the method or composition further comprises the step of diluting the sample prior to partitioning the sample into aliquots.

In some embodiments, aliquots, reaction aliquots or compositions in a collection of compositions are selected from: droplets, microdroplets, droplets in an emulsion, aqueous droplets in oil, aliquots on a solid surface, and aliquots in wells covered with oil.

In some embodiments, the oil comprises a fluorinated oil, a surfactant, and/or a fluorosurfactant.

In some embodiments, the one or more types are haplotypes.

In some embodiments, the nucleic acid polymers are amplicons generated by polymerase chain reaction.

In some embodiments, the nucleic acid polymers are amplicons generated by polymerase chain reaction in the presence of a first and second pair of primers for the first and second genetic loci, respectively, each pair comprising a forward and a reverse primer, and wherein one of the first pair comprises a region of complementarity with one of the second pair, wherein the region of complementarity comprises the second spacing region, or the second spacing region comprises the region of complementarity.

In some embodiments, the distance between the first genetic locus and either of the first pair of primers is no more than 10, 25, 50, 100, 150, 200, 225, 250, 300, 350, 400, 450, or 500 bp, and/or the distance between the second genetic locus and either of the second pair of primers is no more than 10, 25, 50, 100, 150, 200, 225, 250, 300, 350, 400, 450, or 500 bp.

In some embodiments, the region of complementarity is GC-rich.

In some embodiments, the region of complementarity is at least 5, 10, 15, 20, 25, 30, 35, 40, 45, 50 nt long.

In some embodiments, the step of phasing the allelic variants is performed using a method or composition selected from: sequencing, hybridization with probes, hybridization with allele-specific probes, and amplification with allele-specific probes.

In some embodiments, the step of phasing the allelic variants further comprises the step of barcoding the one or more of the nucleic acid polymers prior to sequencing the nucleic acid polymers.

In some embodiments, of phasing the allelic variants further comprises the steps of combining two or more of the aliquots and barcoding the one or more of the nucleic acid polymers prior to sequencing the nucleic acid polymers, wherein the steps of combining two or more of the aliquots and barcoding the one or more of the nucleic acid polymers can be performed in either order.

In some embodiments, in the step of phasing the allelic variants, sequencing is performed using a next generation sequencing technique.

In some embodiments, the organism or individual, the one or more types of nucleic acid templates, or the sample is heterozygous or compound heterozygous at one or more of the first, second or multiple genetic loci.

### Biological Sample

In some embodiments, the present disclosure provides methods for phasing allelic variants of genetic loci on a nucleic acid template in a biological sample. In some embodiments, the present disclosure encompasses a composition comprising a nucleic acid polymer, wherein the polymer comprises two or more genetic loci from a nucleic acid template in a biological sample.

The biological sample can be obtained via any method known in the art.

In some embodiments, a biological sample includes any specimen, tissue or other polynucleotide-containing material obtained from an organism. A biological sample includes, but is not limited to: bile, biopsy tissue including lymph nodes, blood cells, body or cellular fluids, bone marrow, cellular or tissue materials, cerebrospinal fluid, cervical swab samples, exosomes, gastrointestinal tissue, mucus, peripheral blood, plasma, respiratory tissue or exudates, secretions, semen, serum, stool, tissues, and urine. In some embodiments, a biological sample is selected from: tissue, cells, blood, sputum, cheek swab, saliva, skin, urine, a Formalin-fixed, paraffin-embedded tissue sample (FFPE), and a prepared sample. A biological sample can further comprise a diluent, preservative, transport media, and/or other fluid or compound intended to dilute, hold and/or preserve a biological sample. In some embodiments, a biological sample is, comprises, or is derived from any of: a body fluid (e.g., blood, blood plasma, serum, or urine), a cell, a fraction, an organ, or a tissue derived from or isolated from an organism. In some embodiments, a biological sample includes an extra or a portion of a larger biological sample, e.g., sectional portions of an organ or tissue.

In some embodiments, a biological sample is collected via any method known in the art. In some embodiments, a biological sample is collected via buccal swap, fingerstick, venipucture, biopsy, defecation, or urination. In some embodiments, a biological sample further comprises a preservation or other compound or molecule which limits or retards the degradation of nucleic acids in the biological sample. In some embodiments, a biological sample further comprises: acetic acid, alcohol, a buffer, citric acid, a DNase inhibitor, formalin, formaldehyde, a nuclease inhibitor, a RNase inhibitor, SDS, sodium acetate, sodium EDTA, sodium hydroxide, sodium phosphate, and/or Tris HCl. In some embodiments, a biological sample is chopped, cut, dessicated, frozen, snap frozen, sheared, homogenized, and/or refrigerated.

In some embodiments, a biological sample can be obtained from any organism or cell, including but not limited to: a bacterium, virus, yeast and other fungus, plant, or any prokaryote or eukaryote, including any animal or mammal. In some embodiments, a biological sample is a cell or a portion of a cell grown in vitro or capable of growing in vitro. In some embodiments, a biological sample is derived from a living organism. In some embodiments, a biological sample is derived from a deceased organism (e.g., a corpse or dead animal). In some embodiments, a biological sample is of mammalian origin (e.g., derived from a cat, cow or other bovine, dog, donkey, guinea pig, horse, mouse, pig, rabbit, rat or sheep). In some embodiments, a biological sample is derived from a reptile, amphibian or bird (e.g., a chicken or turkey). In some embodiments, a biological sample is derived from a domesticated animal. In some embodiments, a biological sample is from an organism which is used or capable of being used as a source of food for human beings, or as a pet or farm animal. In some embodiments, a biological sample is obtained from a human being.

In some embodiments, a biological sample is obtained from a patient, e.g., a human patient or subject. In some embodiments, the sample is from a patient afflicted with, showing symptoms of, suspected to have, having or suspected of having a genetic predisposition to, or susceptible to a disorder.

In some embodiments, a biological sample is from a patient afflicted with, showing symptoms of, suspected to have, having or suspected of having a genetic predisposition to, or susceptible to a disorder selected from cancer, autoimmune disease, infection, neurological, neuromuscular or neurodegenerative disease, and the first and/or second genetic locus is associated with the disorder.

In some embodiments, a biological sample is from a patient afflicted with, showing symptoms of, suspected to have, having or suspected of having a genetic predisposition to, or susceptible to a cancer, wherein the cancer is selected from: breast cancer, lung cancer, multiple myeloma, ovarian cancer, liver cancer, liver cancer gastric cancer, prostate cancer, acute myeloid leukemia, brain cancer, glioblastoma, non-Hodgkin's lymphoma, leukemia, chronic myeloid leukemia, osteosarcoma, squamous cell carcinoma, and melanoma, solid tumors, primary and metastatic cancers such as renal cell carcinoma, and cancers of the lung (e.g., small cell lung cancer “SCLC” and non-small cell lung cancer “NSCLC”), pancreas, hematopoietic malignancy, glioma, astrocytoma, mesothelioma, colorectal cancers, prostate cancer, osteosarcoma, melanoma, lymphoma (including but not limited to Burkitt's Lymphoma), breast cancer, endometrial cancer, liver cancer, gastric cancer, skin cancer, ovarian cancer and squamous cell cancers of any origin (e.g., lung, head and neck, breast, thyroid, cervix, skin, esophageal, etc.), as well as liquid cancers, e.g., such as leukemias including especially a T-cell leukemia such as acute T-cell leukemia (T-ALL), acute B-cell leukemia (B-ALL), chronic myelogenous leukemia (CML), acute myelogenous leukemia (AML), plasma cell myeloma and multiple myeloma (MM).

In some embodiments, a biological sample is from a patient afflicted with, showing symptoms of, suspected to have, having or suspected of having a genetic predisposition to, or susceptible to an autoimmune disease, the autoimmune disease is selected from Addison's disease, arteriosclerosis, arteriosclerosis, atherosclerosis, atrophic gastritis, autoimmune hemolytic anemia, bullous pemphigoid, chronic active hepatitis, climacterium praecox, discoid lupus erythematosus, Goodpasture's syndrome, Hashimoto's thyroiditis, insulin resistant diabetes, juvenile diabetes, lens-induced uveitis, male infertility, mixed connective tissue disease, multiple sclerosis, myasthenia gravis, paroxysmal hemoglobinuria, pemphigus vulgaris, pernicious anemia, polymyositis, primary biliary liver cirrhosis, primary myxedema, rapidly progressive glomerulonephritis, scleroderma, Sjogren syndrome, sudden thrombocytopenic purpura, sympathetic phlebitis, systemic lupus erythematosus, thyrotoxicosis, and ulcerative colitis.

In some embodiments, a biological sample is from a patient afflicted with, showing symptoms of, suspected to have, having or suspected of having a genetic predisposition to, or susceptible to an infection, wherein the infection is selected from Acinetobacter infection, Actinomycosis, African sleeping sickness (African trypanosomiasis), AIDS (Acquired immunodeficiency syndrome), Amebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas Disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Clonorchiasis, Clostridium difficile colitis, Coccidioidomycosis, Colorado tick fever (CTF), Common cold (Acute viral rhinopharyngitis; Acute coryza), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum, Exanthem subitum, Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr Virus Infectious Mononucleosis (Mono), Influenza (flu), Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease (PID), Pertussis (Whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Subacute sclerosing panencephalitis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans (OLM)), Toxocariasis (Visceral Larva Migrans (VLM)), Trachoma, Toxoplasmosis, Trichinosis, Trichomoniasis, Trichuriasis (Whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile Fever, White piedra (Tinea blanca), Yersinia pseudotuberculosis infection, Yersiniosis, Yellow fever, or Zygomycosis; or is caused or associated with Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma species, Angiostrongylus, Anisakis, Bacillus anthracis, Arcanobacterium haemolyticum, Junin virus, Ascaris lumbricoides, Aspergillus species, Astroviridae family, Babesia species, Bacillus cereus, multiple bacteria, List of bacterial vaginosis microbiota, Bacteroides species, Balantidium coli, Bartonella, Baylisascaris species, BK virus, Piedraia hortae, Blastocystis species, Blastomyces dermatitidis, Machupo virus, Clostridium botulinum; Note: Botulism is not an infection by Clostridium botulinum but caused by the intake of botulinum toxin, Sabia, Brucella species, the bacterial family Enterobacteriaceae, usually Burkholderia cepacia and other Burkholderia species, Mycobacterium ulcerans, Caliciviridae family, Campylobacter species, usually Candida albicans and other Candida species, Intestinal disease by Capillaria philippinensis, hepatic disease by Capillaria hepatica and pulmonary disease by Capillaria aerophila, Bartonella bacilliformis, Bartonella henselae, usually Group A Streptococcus and Staphylococcus, Trypanosoma cruzi, Haemophilus ducreyi, Varicella zoster virus (VZV), Alphavirus, Chlamydia trachomatis, Chlamydophila pneumoniae, Vibrio cholerae, usually Fonsecaea pedrosoi, Clonorchis sinensis, Clostridium difficile, Coccidioides immitis and Coccidioides posadasii, Colorado tick fever virus (CTFV), usually rhinoviruses and coronaviruses, PRNP, Crimean-Congo hemorrhagic fever virus, Cryptococcus neoformans, Cryptosporidium species, usually Ancylostoma braziliense; multiple other parasites, Cyclospora cayetanensis, Taenia solium, Cytomegalovirus, Dengue viruses (DEN-1, DEN-2, DEN-3 and DEN-4)—Flaviviruses, Green algae Desmodesmus armatus, Dientamoeba fragilis, Corynebacterium diphtheriae, Diphyllobothrium, Dracunculus medinensis, Ebolavirus (EBOV), Echinococcus species, Ehrlichia species, Enterobius vermicularis, Enterococcus species, Enterovirus species, Rickettsia prowazekii, Parvovirus B19, Human herpesvirus 6 (HHV-6) and Human herpesvirus 7 (HHV-7), Fasciola hepatica and Fasciola gigantica, Fasciolopsis buski, PRNP, Filarioidea superfamily, Clostridium perfringens, multiple, Fusobacterium species, usually Clostridium perfringens; other Clostridium species, Geotrichum candidum, PRNP, Giardia lamblia, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71 (EV71), Sin Nombre virus, Heartland virus, Helicobacter pylori, Escherichia coli 0157:H7, O111 and O104:H4, Bunyaviridae family, Hepatitis A virus, Hepatitis B virus, Hepatitis C virus, Hepatitis D Virus, Hepatitis E virus, Herpes simplex virus 1 and 2 (HSV-1 and HSV-2), Histoplasma capsulatum, Ancylostoma duodenale and Necator americanus, Human bocavirus (HBoV), Ehrlichia ewingii, Anaplasma phagocytophilum, Human metapneumovirus (hMPV), Ehrlichia chaffeensis, Human papillomavirus (HPV), Human parainfluenza viruses (HPIV), Hymenolepis nana and Hymenolepis diminuta, Epstein-Barr Virus (EBV), Orthomyxoviridae family, Isospora belli, unknown; evidence supports that it is infectious, multiple, Kingella kingae, PRNP, Lassa virus, Legionella pneumophila, Legionella pneumophila, Leishmania species, Mycobacterium leprae and Mycobacterium lepromatosis, Leptospira species, Listeria monocytogenes, Borrelia burgdorferi, Borrelia garinii, and Borrelia afzelii, Wuchereria bancrofti and Brugia malayi, Lymphocytic choriomeningitis virus (LCMV), Plasmodium species, Marburg virus, Measles virus, Middle East respiratory syndrome coronavirus, Burkholderia pseudomallei, multiple, Neisseria meningitidis, usually Metagonimus yokagawai, Microsporidia phylum, Molluscum contagiosum virus (MCV), Monkeypox virus, Mumps virus, Rickettsia typhi, Mycoplasma pneumoniae, numerous species of bacteria (Actinomycetoma) and fungi (Eumycetoma), parasitic dipterous fly larvae, most commonly Chlamydia trachomatis and Neisseria gonorrhoeae, PRNP, usually Nocardia asteroides and other Nocardia species, Onchocerca volvulus, Opisthorchis viverrini and Opisthorchis felineus, Paracoccidioides brasiliensis, usually Paragonimus westermani and other Paragonimus species, Pasteurella species, Pediculus humanus capitis, Pediculus humanus corporis, Phthirus pubis, multiple, Bordetella pertussis, Yersinia pestis, Streptococcus pneumoniae, Pneumocystis jirovecii, multiple, Poliovirus, Prevotella species, usually Naegleria fowleri, JC virus, Chlamydophila psittaci, Coxiella burnetii, Rabies virus, Borrelia hermsii, Borrelia recurrentis, and other Borrelia species, Respiratory syncytial virus (RSV), Rhinosporidium seeberi, Rhinovirus, Rickettsia species, Rickettsia akari, Rift Valley fever virus, Rickettsia rickettsii, Rotavirus, Rubella virus, Salmonella species, SARS coronavirus, Sarcoptes scabiei, Schistosoma species, multiple, Shigella species, Varicella zoster virus (VZV), Variola major or Variola minor, Sporothrix schenckii, Staphylococcus species, Staphylococcus species, Strongyloides stercoralis, Measles virus, Treponema pallidum, Taenia species, Clostridium tetani, usually Trichophyton species, usually Trichophyton tonsurans, usually Trichophyton species, usually Epidermophyton floccosum, Trichophyton rubrum, and Trichophyton mentagrophytes, Trichophyton rubrum, usually Hortaea werneckii, usually Trichophyton species, usually Trichophyton species, Malassezia species, Toxocara canis or Toxocara cati, Toxocara canis or Toxocara cati, Chlamydia trachomatis, Toxoplasma gondii, Trichinella spiralis, Trichomonas vaginalis, Trichuris trichiura, usually Mycobacterium tuberculosis, Francisella tularensis, Salmonella enterica subsp. enterica, serovar typhi, Rickettsia, Ureaplasma urealyticum, Coccidioides immitis or Coccidioides posadasii, Venezuelan equine encephalitis virus, Guanarito virus, Vibrio vulnificus, Vibrio parahaemolyticus, multiple viruses, West Nile virus, Trichosporon beigelii, Yersinia pseudotuberculosis, Yersinia enterocolitica, Yellow fever virus, Mucorales order (Mucormycosis), or Entomophthorales order (Entomophthoramycosis).

In some embodiments, a disorder is selected from: inflammatory and autoimmune diseases, such as systemic lupus erythematosus, Hashimoto's disease, rheumatoid arthritis, graft-versus-host disease, Sjogren's syndrome, pernicious anemia, Addison disease, scleroderma, Goodpasture's syndrome, Crohn's disease, autoimmune hemolytic anemia, sterility, myasthenia gravis, multiple sclerosis, Basedow's disease, thrombotic throbocytopenia, thrombopenia purpurea, insulin-dependent diabetes mellitus, allergy; asthma, atopic disease; arteriosclerosis; myocarditis; cardiomyopathy; globerula nephritis; and hypoplastic anemia.

In some embodiments, a disorder is selected from: Obesity, Childhood Obesity, Attention Deficit Hyperactivity Disorder, Headache, migraine, Asthma, Asthma (Pediatric), Depression, Hypertension, Food allergy, Anxiety Disorder, Substance Use Disorder, Speech Defect, HPV infection, Autism Spectrum Disorder, Headache, chronic daily, Intellectual Disability, Tourette syndrome, Premature Infant (e.g., <27 weeks), Celiac Disease, Traumatic Brain Injury (cum. incidence), Seizure Disorder, Hearing Loss, Congenital Heart Defects, all, Cerebral Palsy, Familial Hypercholesterolemia (heterozygote), Diabetes Mellitus, Type I, in children, Ventricular Septal Defect, Cancer, Down Syndrome, Neural Tube Defects, Hearing loss or deafness, congenital, Hearing loss, serious (e.g., >40 db, bilat.), Visual Impairment (e.g., worse than 20/70), Cleft Lip, with or without cleft palate, Juvenile Arthritis, XXY (Klinefelter) Syndrome, Club Foot, Stroke, Idiopathic Thrombocytopenic Purpura, Fetal Alcohol Syndrome, Hydrocephalus, Inflammatory Bowel Disease, Rheumatic Heart Disease, Rectal & Large Intestinal Atresia/Stenosis, d-Transposition of the Great Arteries, Congenital Hypothyroidism, Crohn's Disease, Patent Ductus Arteriosus, Atrial Septal Defect, All Tandem Mass-screened Conditions, Tetralogy of Fallot, Reduction Defects, upper limbs, Gastroschisis, Coarctation of the Aorta, Pulmonary Valve Stenosis, Spinal Cord Injury, Turner Syndrome, Diaphragmatic Hernia, Growth Hormone Deficiency, Childhood Absence Epilepsy, Aortic Valve Stenosis, Amino Acid Disorders—total, Lennox-Gastaut syndrome, Neurofibromatosis, Type I, 22q 1.2 deletion syndrome, Hypoplastic Left Heart Syndrome, Esophageal/Tracheoesophageal Atresia, Trisomy 18, Sickle Cell Disease, Cystic Fibrosis, Anophthalmia/Microphthalmia, Infantile Spasms, Hirschsprung Disease, Fragile X Syndrome, Charcot-Marie-Tooth Disease, Spinal Muscular Atrophy (SMA), Duchenne Muscular Dystrophy, muscular dystrophy, Osteogenesis Imperfecta, Trisomy 13, Leukodystrophies, Hypoplastic Right Ventricle, Romano-Ward Syndrome, Deafness, Phenylketonuria, Fatty Acid Oxidation Disorders—total, Single Ventricle, Total Anomalous Pulmonary Venous Return, Truncus Arteriosus, Chronic Renal Failure, Hemophilia (A & B), Organic Acidurias—total, Marfan Syndrome, Congenital Adrenal Hyperplasia, Primary Ciliary Dyskinesia (immotile cilia), MCADD, Angelman Syndrome, Retinoblastoma, Dilated Cardiomyopathy, Hemophilia A, Tuberous Sclerosis, Polycystic Kidney Disease, autsomal recessive, Glycogen Metabolism/Storage Diseases, Huntington Disease, Prader Willi Syndrome, Rett Syndrome, VLCADD, Acute Lymphoblastic Leukemia (ALL), Cancer, brain & CNS, Friedreich's Ataxia, Myotonic Muscular Dystrophy, Cornelia de Lange Syndrome, Fabry Disease, Biotinidase deficiency, Hemophilia B, Hodgkin Lymphoma, Ataxia-Telangiectasia, Non-Hodgkin Lymphoma, Hurler Syndrome (Mucopolysaccharidosis type I), Cancer, bone & joint, Tyrosinemia type 1, Neuroblastoma, Acute Myeloid Leukemia (AML), Cancer, kidney & renal pelvis, Wilms Tumor, Maple Syrup Urine Disease, LCHADD/TFP Deficiency, Isovaleric acidemia, Behcet's Disease, Homocystinuria, Chronic Granulomatous Disease, Myasthenia Gravis, Familial Hypercholesterolemia (e.g., homozygote), and Arginase deficiency.

In some embodiments, a neurological, neurodegenerative, or neuromuscular disease is: Abulia, Agraphia, Alcoholism, Alexia, Alien hand syndrome, Alzheimer's disease, Amaurosis fugax, Amnesia, Amyotrophic lateral sclerosis (ALS), Aneurysm, Angelman syndrome, Aphasia, Apraxia, Arachnoiditis, Arnold-Chiari malformation, Asperger syndrome, Ataxia, Attention deficit hyperactivity disorder, ATR-16 syndrome, Auditory processing disorder, Autism spectrum, Behcets disease, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Cockayne syndrome, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Disorders of consciousness, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Essential tremor, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barre syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lou Gehrig's disease, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor Neurone Disease, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behcet's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Phantom limb/Phantom pain, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Vestibular schwannoma (Acoustic neuroma), Von Hippel-Lindau disease (VHL), Viliuisk Encephalomyelitis (VE), Wallenberg's syndrome, West syndrome, Whiplash, Williams syndrome, Wilson's disease, or Zellweger syndrome.

In some embodiments, a neurological, neurodegenerative, or neuromuscular disease is: A Muscular Dystrophy, Becker Muscular Dystrophy (BMD), Congenital Muscular Dystrophy (CMD), Distal Muscular Dystrophy (DD) (Miyoshi), Duchenne Muscular Dystrophy (DMD) (Pseudohypertrophic), Emery-Dreifuss Muscular Dystrophy (EDMD), Facioscapulohumeral Muscular Dystrophy (FSH) (FSHD) (Landouzy-Dejerine), Limb-Girdle Muscular Dystrophy (LGMD), Myotonic Muscular Dystrophy (MMD) (DM) (Steinert Disease), Oculopharyngeal Muscular Dystrophy (OPDM), A Motor Neuron Condition, Amyotrophic Lateral Sclerosis (ALS) (Lou Gehrig's Disease) (MND) (Motor Neurone Disease), Spinal Bulbar Muscle Atrophy (SBMA) (X-Linked SBMA) (Kennedy Disease), Spinal Muscle Atrophy Type 1 (SMA1) (Werdnig-Hoffman Disease), Spinal Muscle Atrophy Type 2 (SMA2), Spinal Muscle Atrophy Type 3 (SMA3) (Kugelberg-Welander Disease), A Metabolic Muscle Condition, Acid Maltase Deficiency (AMD) (Pompe Disease), Phosphorylase Deficiency (Myphosphorylase Deficiency) (McArdle Disease) (MPD) (PYGM), Phosphofructokinase Deficiency (Tauri Disease), Debrancher Enzyme Deficiency (DBD) (Cori Disease) (Forbes Disease), Mytochondrial Myopathies, Carnitine Deficiency (CD), Carnitine Palmityl Transferase Deficiency (CPT), Phosphoglycerae Kinase Deficiency, Phosphoglycerate Mutase Deficiency, Lactate Dheydrogenase Deficiency (LDHA), Myoadenylate Deaminase Deficiency, A Condition of the Peripheral Nerve, Charcot-Marie-Tooth Disease (CMT) (Peroneal Muscular Atrophy) (PMA) (Hereditary Motor and Sensory Meuropathy) (HMSN), Friedreich's Ataxia (FA), Dejerine-Sottas Disease (CMT4F), A Condition of the Neuromuscular Junction, Myasthenia Gravis (MG), Lambert-Eaton Myasthenic Syndrome (LES), Congenital Myasthenic Sydnromes (CMS), A Neuromuscular Myopathy, Myotonia Congenita (MC) (Thomsen's Disease) (Becker's Disease), Paramyotonia Congenita (PC), Central Core Disease (CCD), Nemaline Myopathy (NM), Myotubular Myopathy (MTM), Centronuclear Myopathy (CNM), Periodic Paralysis (PP) (Hyperkalemic Periodic Paralysis), Hyperthyroid Myopathy (MYPTM), Hypothyroid Myopathy (HPOTM), Dermatomyositis (DM), Polymyositis (PM), or Inclusion Body Myositis (IBM).

A biological sample derived from any organism or cell, including human patients afflicted with, showing symptoms of, suspected to have, having or suspected of having a genetic predisposition to, or susceptible to any of various diseases, and obtained using any method known in the art, can be used in the methods and compositions of the present disclosure.

### Nucleic Acid Template

The present disclosure provides, among other things, methods for phasing allelic variants of genetic loci on a nucleic acid template. In some embodiments, the present disclosure provides compositions and methods pertaining to nucleic acids comprising two or more genetic loci. Provided nucleic acids are useful for many purposes, including, but not limited to, phasing.

In some embodiments, it is known that two (or more) alleles of interest in a nucleic acid template each comprise different sequences. For example, sequencing can reveal that each of two alleles on a nucleic acid template are heterologous. For example, one allele can be either of the allelic variants A or a, while a second allele can be either of the allelic variants B or b. The present disclosure provides methods and compositions, including those related to phasing of the allelic variants, thus determining, for example, if (1) one chromosome comprises the alleles A and B and the other a and b; or (2) one chromosome comprises the alleles A and b and the other a and B, in a nucleic acid template.

In some embodiments, a nucleic acid template comprises, is, or is derived from any of: a chromosome, chromosomal fragment, genomic DNA, mRNA or cDNA.

In some embodiments, a nucleic acid template comprises a first and a second genetic locus, with a spacing region between the loci. A nucleic acid template can be in a biological sample which is prepared (e.g., sheared, homogenized, frozen, etc.) in such a way that at least one copy of the template remains in the sample wherein the copy comprises both the first and second genetic loci. For example, in some embodiments, a biological sample comprising a chromosome is not sheared or homogenized, as such action may break the spacing region and physically separate the first and second loci. In some embodiments, a biological sample is lightly sheared or homogenized, such that a template is partially damaged (e.g., one or both ends are shortened or broken), but at least one copy of the template remains comprising both the first and second genetic loci.

In some embodiments, a method or composition of the present disclosure relates to a nucleic acid template comprising three or more genetic loci, or comprising the step of phasing three or more genetic loci. In some embodiments, a biological sample comprising a nucleic template is prepared in such a way that at least one copy of the template remains which comprises all the genetic loci to be phased.

In some embodiments, the length of the first spacing region at least 0.01, 0.025, 0.05, 0.075, 0.1, 0.25, 0.5, 0.75, 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, 1000 kb.

Various nucleic acid templates as described herein or known in the art can be used in methods and compositions of the present disclosure.

### Heterologous Nature of Certain Nucleic Acid Template

In some embodiments, it is known that two (or more) alleles of interest in a nucleic acid template each comprise different sequences.

In some embodiments, a nucleic acid template is determined to be heterozygous at two (or more) alleles of interested, wherein the determination is performed using an assay known in the art.

In some embodiments, a nucleic acid template is determined to be heterozygous at two (or more) alleles of interest, wherein the determination is performed using an PCR based genotyping assay.

In some embodiments, in a method of phasing allelic variants of a first and a second genetic locus on a nucleic acid template, the nucleic acid template is determined to be heterozygous at the first and second genetic locus, wherein the determination is performed using an PCR based genotyping assay.

The determination that a nucleic acid template is heterologous can be performed using any method known in the art.

In some embodiments, sequencing is performed to determine if a nucleic acid template is heterozygous at two or more alleles of interest.

In some embodiments, a PCR based genotyping assay or amplification using allele-specific primers is performed to determine if a nucleic acid template is heterozygous at two or more alleles of interest.

In some embodiments, a PCR based genotyping assay or amplification using allele-specific primers is an ARMS (Amplification Refractory Mutation System) assay.

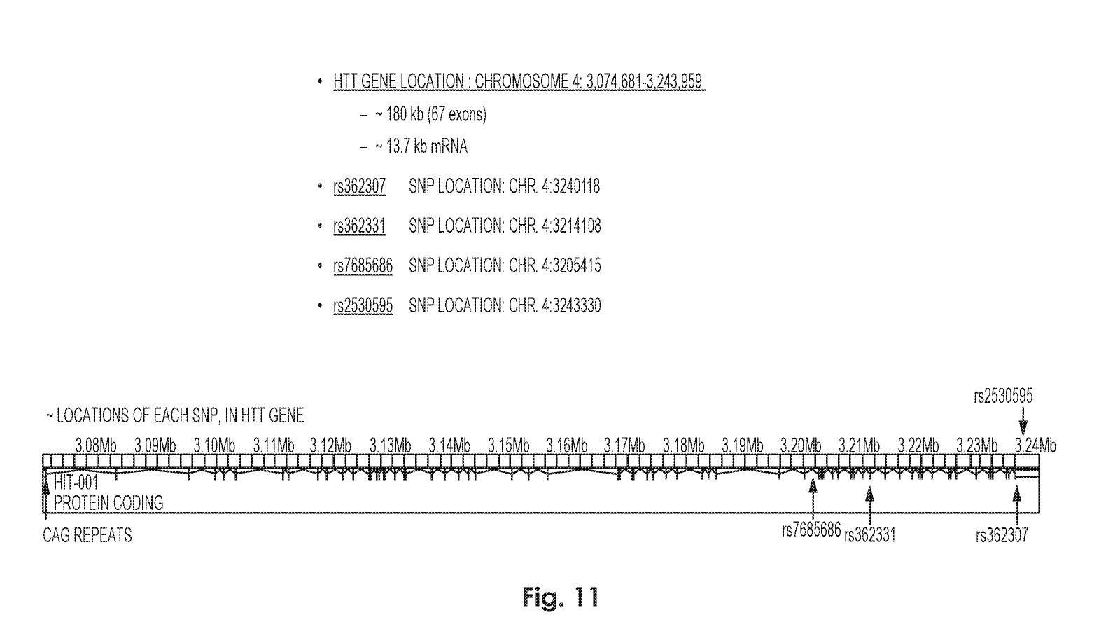

In some embodiments, an ARMS assay is performed to determine if a nucleic acid template is heterozygous at two or more alleles of interest. A non-limiting example of an ARMS assay is shown in FIGS. 11 to 15A and 15B.

In this non-limiting example of an ARMS assay, alleles of interest in the Huntingtin gene were examined: SNP1, rs362307; SNP2, rs362331; SNP3, rs2530595; and SNP4, rs7685686 (see FIG. 11). The ARMS assay is based on the use of sequence-specific PCR primers, allow amplification of test DNA only when the target allele is contained within the sample. The assay employs two pairs of primers to amplify two alleles in one PCR reaction. The primers are designed such that the two primer pairs overlap at the allele of interest (e.g., the SNP location) but each match perfectly to only one of the possible alleles (e.g., SNPs). The two primer pairs are also designed such that their PCR products are of a significantly different length allowing for easily distinguishable bands by gel electrophoresis.

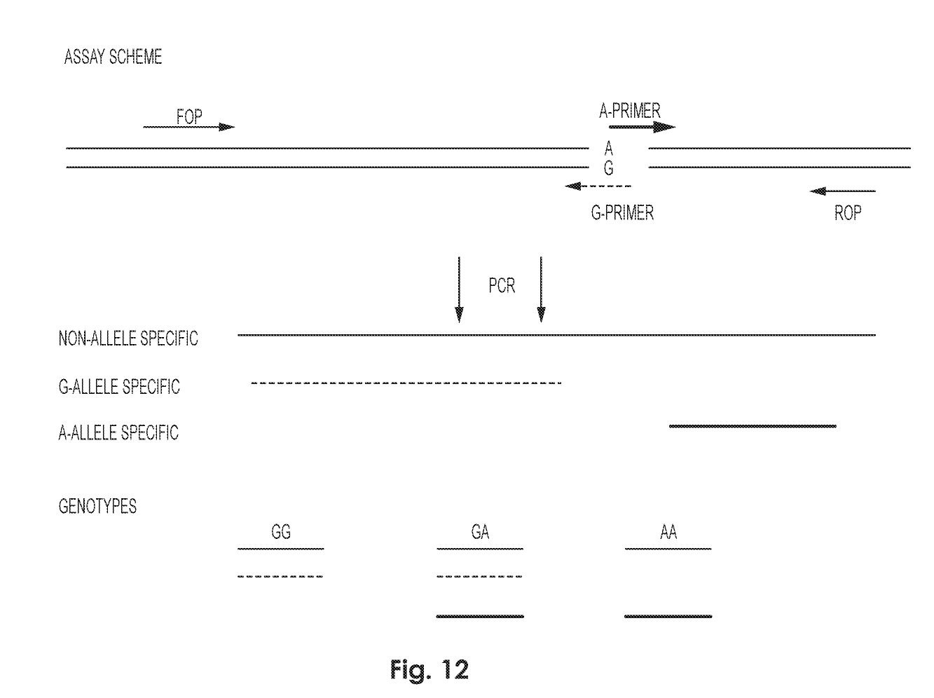

A cartoon of a non-limiting example of an ARMS assay is shown in FIG. 12. Two inner primers recognize either the A or G allele (A-primer and G-primer, respectively). FOP (Forward Outer Primer) and ROP (Reverse Outer Primer) are also used. Amplification yields fragments of different lengths. In all cases, a long amplicon results from use of the FOP and ROP. In the presence of the A allele, an amplicon is produced using the A-primer and ROP. In the presence of the G allele, an amplicon is produced using the FOP and the G-primer. The FOP is further from the allele of interest than the ROP; thus, the amplicon representing the A allele is a different length than the amplicon representing the G allele. The A and G amplicons can thus be distinguished in electrophoresis. The presence of bands corresponding to the expected lengths of the A and G amplicons indicates that the test nucleic is heterozygous (A and G) at allele of interest. If only a band of the expected length of the A amplicon appears, then the test nucleic acid is homozygous A at this allele. If only a band of the expected length of the G amplicon appears, then the test nucleic acid is homozygous G at this allele.

In a non-limiting example, an ARMS assay was performed to determine if various cell lines were heterozygous at an allele SNP2 (rs362331, T or C) of the Huntingtin gene.

In this non-limiting example, the following primers were used:

Forward inner primer (T allele):
TTTGTGACCCACGCCTGCTCCCTCAGCT
GC: 60.7% Tm: 68.6° C. DeltaG: −58.78 kcal/mole
Reverse inner primer (C allele):
CCGGCCTCCAGGATGAAGTGCACACATTG
GC: 58.6% Tm: 66.1° C. DeltaG: −59.3 kcal/mole
Forward outer primer (5′-3′):
TCCCGCTGAGTCTGGATCTCCAGGCAGG
GC: 64.3% Tm: 67.9° C. DeltaG: −57.98 kcal/mole
Reverse outer primer (5′-3′):
GATGGGGCCCAGCCCTTCCTGATGCATA
GC: 60.7% Tm: 67.3° C. DeltaG: −60.59 kcal/mole

Product [amplicon] size for T allele: 209 nt

Product [amplicon] size for C allele: 148 nt

Product [amplicon] size of two outer primers: 300 nt

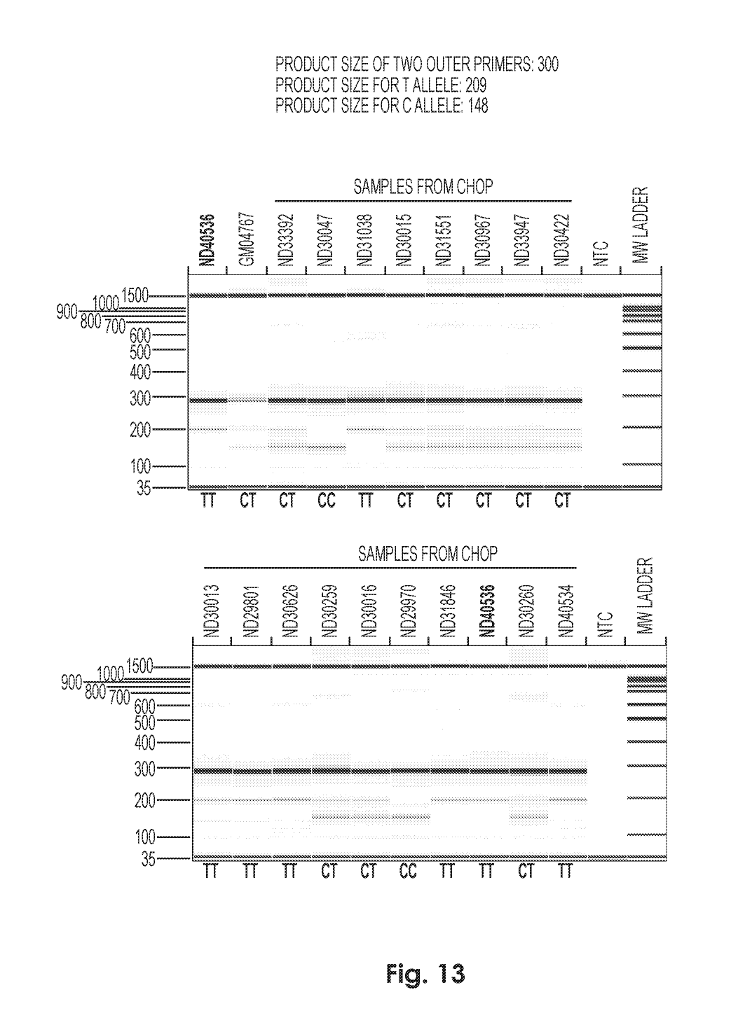

FIG. 13 shows genotyping results for 19 cell lines for Huntingtin SNP2 rs362331. The presence of only the T amplicon band indicates that a particular cell line is homozygous T at this position (TT); the presence of only the C amplicon band indicates that a particular cell line is homozygous C at this position (CC); the presence of both the T and C amplicon bands indicates that a particular cell line is heterozygous at this position (CT).

In another non-limiting example, an ARMS assay was performed to determine if various cell lines were heterozygous at SNP3 (rs2530595, G or A) of the Huntingtin gene.

In this non-limiting example, the following primers were used:

Forward inner primer (G allele):
AGCAGCTGTGGTCCCGGGTCCTCCACG
GC: 70.4% Tm: 70.8° C. DeltaG: −59.29 kcal/mole
Reverse inner primer (A allele):
TGTCCCTCCCCCGCTTCCTCCCTCGGT
GC: 70.4% Tm: 72.4° C. DeltaG: −61.91 kcal/mole
Forward outer primer (5′-3′):
GAGCGGCCCCTGAACCTTCCAGGTTGCC
GC: 67.9% Tm: 69.8° C. DeltaG: −63.08 kcal/mole
Reverse outer primer (5′-3′):
TAACTCGGTGTGTGGCCGCCTGGCAGGT
GC: 64.3% Tm: 70.3° C. DeltaG: −60.81 kcal/mole

Product size for G allele: 198

Product size for A allele: 264

Product size of two outer primers: 408

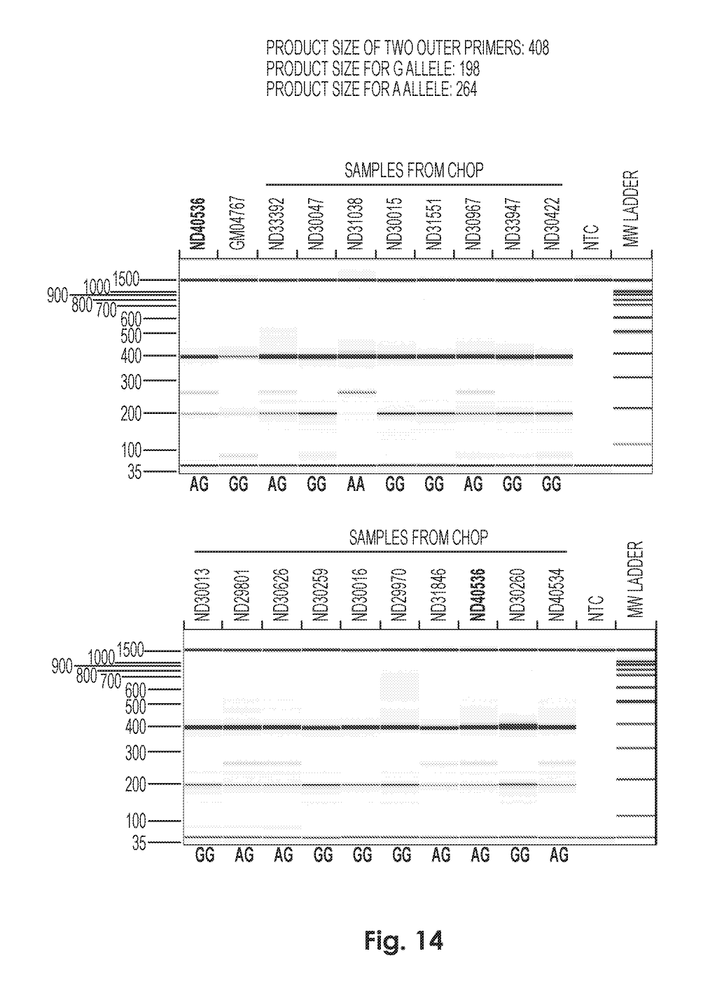

FIG. 14 shows genotyping results for 19 cell lines for Huntingtin SNP3 (rs2530595, G or A). The presence of only the G amplicon band indicates that a particular cell line is homozygous G at this position (GG); the presence of only the A amplicon band indicates that a particular cell line is homozygous A at this position (AA); the presence of both the G and A amplicon bands indicates that a particular cell line is heterozygous at this position (GA).

In another non-limiting example, an ARMS assay was performed to determine if various cell lines were heterozygous at SNP1 (rs362307, G or A) of the Huntingtin gene.

In this non-limiting example, the following primers were used:

| | |
|---|---|
| FIP (G allele): | CTCGGTGGAGGCAGGGCACAAGGTCG |
| RIP (A allele): | CTGGGGCCGGAGCCTTTGGAAGTCGGT |
| FOP: | CCCACCAGGACTGCAGACACTCCCTGCC |
| ROP: | AGCTCGACCGCAGGGCCTTCCAGTCTGT |

Product size for G allele: 198

Product size for A allele: 178

Product size of two outer primers: 323

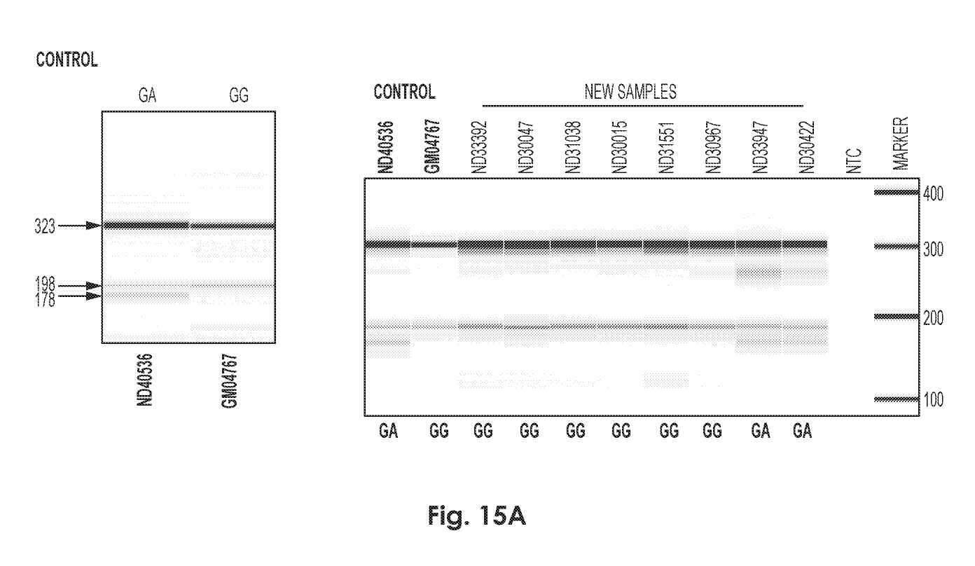

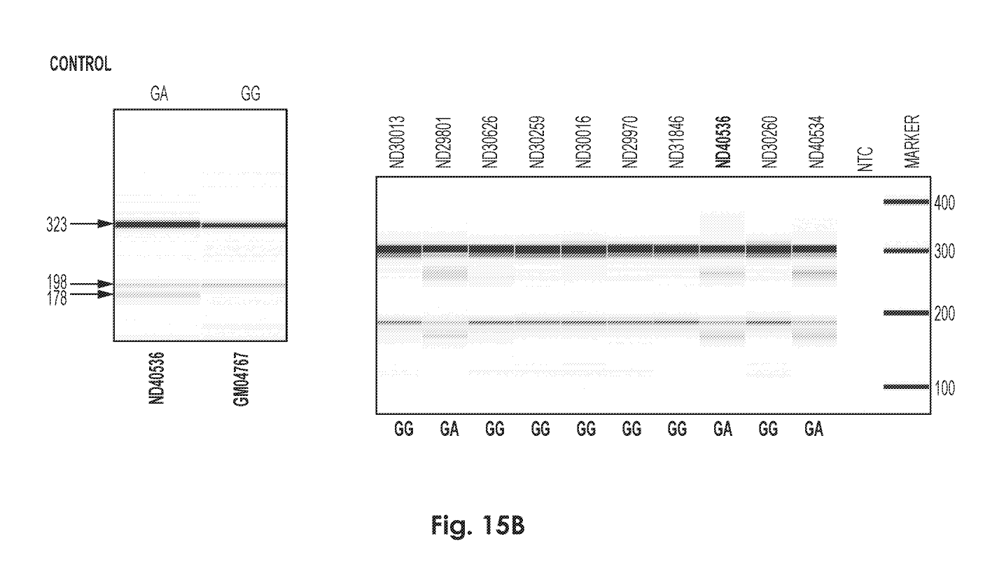

FIGS. 15A and 15B show genotyping results for 19 cell lines for Huntingtin SNP1 (rs362307, G or A). The presence of only the G amplicon band indicates that a particular cell line is homozygous G at this position (GG); the presence of only the A amplicon band indicates that a particular cell line is homozygous A at this position (AA); the presence of both the G and A amplicon bands indicates that a particular cell line is heterozygous at this position (GA).

Without wishing to be bound by any particular theory, the present disclosure notes that the ARMS assay is: highly sensitive and reliable; fast and easy to perform, with results available in less than 3 hours; and inexpensive. The ARMS assay requires no special equipment other than thermocyclers and no post-PCR treatment other than electrophoresis.

Sequencing, the ARMS assay, or any other method known in the art can be used to determine if a nucleic acid template is heterozygous at various alleles of interest.

Partitioning into Aliquots

The present disclosure provides, among other things, methods for phasing allelic variants of genetic loci on a nucleic acid template in a biological sample. In some embodiments, the present disclosure provides compositions and methods pertaining to nucleic acids comprising two or more genetic loci. In some embodiments, provided nucleic acids are useful for many purposes, including, but not limited to, phasing. In some embodiments, the method comprises the step of, and a provided nucleic acid is produced via a method comprising a step of partitioning a biological sample into a plurality of aliquots, wherein generally each aliquot comprises no more than one copy of the nucleic acid template.

A biological sample can comprise multiple copies of a nucleic acid template. In some embodiments, a biological sample can comprise, for example, copies of corresponding chromosomes from each parent. The step of partitioning the sample into aliquots separates the multiple copies, so that each aliquot comprises in general no more than one copy of the nucleic acid template. Thus, in one non-limiting example, a chromosome from the mother is physically separated from a corresponding chromosome from the father.

The process of partitioning can comprise, in some embodiments, physically dividing the biological sample into a plurality of volumes. This can be accomplished using any method known in the art.

As a non-limiting example, a biological sample can be highly diluted prior to partitioning. Such dilution can, in some cases, improve the partitioning such that a plurality of aliquots comprises no more than one copy of a template. The present disclosure notes that such partitioning can, in some embodiments, produce many aliquots which comprize zero copies of the template, which aliquots are irrelevant to the present methods and compositions.

In some embodiments of a method or composition of the present disclosure, multiple droplets contain zero copies of a target nucleic acid template, some droplets one copy, and some droplets comprise multiple copies (corresponding to limiting or terminal dilution, respectively, as defined above). In some embodiments, the distribution of copies within droplets obeys the Poisson distribution. In some embodiments, the number of droplets comprising exactly one copy of a nucleic acid template is greater than the number of droplets comprising multiple copies.

In some embodiments, aliquots, reaction aliquots or compositions in a collection of compositions are selected from: droplets, microdroplets, droplets in an emulsion, aqueous droplets in oil, aliquots on a solid surface, and aliquots in wells covered with oil.

In some embodiments, partitioning comprises a step of forming an emulsion. In some embodiments, partitioning comprises a step of forming droplets in an emulsion. In some embodiments, partitioning comprises the step of passing a biological sample, which can be in a liquid form, through a small orifice, such that monodisperse droplets are generated. In some embodiments, partitioning comprises the step of passing a biological sample, which can be in a liquid form, through a small orifice, such that monodisperse droplets are generated and dispersed into a medium in which they do not dissolve. In some embodiments, a medium is an oil.

In some embodiments, droplets are aqueous droplets surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910.

In some embodiments, the droplets may be coated with a surfactant or a mixture of surfactants. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

In some embodiments, droplets are about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 micrometers in diameter. In some embodiments, droplets are no more than about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 micrometers in diameter.

In some embodiments, any method known in the art of partitioning a biological sample into a plurality of aliquots, wherein at least one aliquot comprises one copy of a nucleic acid template, can be used.

### Generating a Plurality of Nucleic Acid Polymers Based on a Nucleic Acid Template

The present disclosure encompasses methods for phasing allelic variants of genetic loci on a nucleic acid template in a biological sample; in some embodiments, the methods comprise the step of generating nucleic acid polymers from the nucleic acid template. The present disclosure also encompasses compositions and methods pertaining to nucleic acids comprising two or more genetic loci. These nucleic acids are useful for many purposes, including, but not limited to, phasing. Phasing of allelic variants in a nucleic acid polymer also serves to phase the allelic variants on the nucleic acid template.

In some embodiments, a nucleic acid template comprises a first and a second genetic locus, with a spacing region interposed between the loci. In some embodiments, the spacing region can be very long or comprise a structure such as a localized amplification, hairpin-loops structures or repeats, which can make difficult methods of phasing comprising the step of simply sequencing the portion of the nucleic acid template comprising the loci of interest. In some embodiments, a nucleic acid polymer based on a nucleic acid template comprises a first and a second genetic locus, but the spacing region between the loci on the polymer differs from the corresponding spacing region on the template. In some embodiments, the spacing region on the polymer eliminates or shortens a sequence on the template which makes difficult methods of phasing the genetic loci. In some embodiments, a nucleic acid polymer is produced from a nucleic acid template which comprises a first and second genetic loci, but the spacing region between the loci is altered (e.g., shortened), thus allowing the phasing of the genetic loci. In some embodiments, a nucleic acid polymer is produced from a nucleic acid template via a method comprising a step of amplification using multiple pairs of primers, wherein a pair of primers flanks a first genetic locus, and another pair of primers flanks another genetic locus, and wherein one primer of a pair of primers is capable of annealing in a sequence-specific manner to a primer of another pair of primers, and wherein the nucleic acid polymer is produced from a nucleic acid template which comprises a first and second genetic loci, but the spacing region between the loci is altered (e.g., shortened), thus allowing the phasing of the genetic loci.

As a non-limiting example, in a nucleic acid template, a first and second genetic loci can be very far apart, for example, 10 kb, which distance makes them difficult to sequence. In some embodiments, the spacing region between the first and second genetic loci is sufficiently shortened on the polymer to allow sequencing of the loci.

In some embodiments, multiple copies of a nucleic acid template in a sample are separated from each in the step of partitioning the sample into aliquots. Thus, in some embodiments, a nucleic acid polymer is produced from a single copy of a nucleic acid template, and is not, for example, a chimera derived from multiple copies. In some embodiments, a nucleic acid polymer is produced from a single copy of a nucleic acid template and not, for example, from one nucleic acid template comprising one allele of a first genetic locus and from another nucleic acid template which can comprise an allele of the second genetic locus.

In some embodiments, a nucleic acid polymer is generated via a method comprising the step of amplification. In some embodiments, amplification comprises the step of generating, from at least one aliquot containing one type of the template, a plurality of nucleic acid polymers based on the template, wherein at least one of the nucleic acid polymers comprises the first and the second genetic locus and a second spacing region that is different from the first spacing region.

In some embodiments, amplication comprises a step comprising PCR.

In some embodiments, the amplification reaction is the polymerase chain reaction. In some embodiments, polymerase chain reaction (PCR) includes methods described by K. B. Mullis (U.S. Pat. Nos. 4,683,195 and 4,683,202) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence.

To effect amplification, primers are annealed to their complementary sequence within the target molecule. Following annealing, the primers are extended with a polymerase so as to form a new pair of complementary strands. The steps of denaturation, primer annealing and polymerase extension can be repeated many times (i.e., denaturation, annealing and extension constitute one cycle; there can be numerous cycles) to obtain a high concentration of an amplified segment of a desired target sequence. The length of the amplified segment of the desired target sequence is determined by relative positions of the primers with respect to each other and by cycling parameters, and therefore, this length is a controllable parameter.

Methods for performing PCR in droplets are shown for example in Link et al. (U.S. patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc.

In some embodiments, amplication of two or more genetic loci on a nucleic acid template produces a nucleic acid polymer which comprises the two or more genetic loci. In some embodiments, amplication of two or more genetic loci on a nucleic acid template produces a nucleic acid polymer which comprises the two or more genetic loci, wherein the spacing region(s) between the loci on the polymer is different from the corresponding spacing region(s) on the template.

In some embodiments, a nucleic acid polymer is generated via amplification of two or more genetic loci on the template using pairs of primers or probes.

In a non-limiting example, a template can comprise an upstream genetic locus and a downstream genetic locus, as produced via amplification in the presence of the appropriate primers. A pair of primers (including a forward and a reverse primer) can be designed and provided, which flank the upstream genetic locus. Another pair of primers (including a forward and a reverse primer) can be designed and provided, which flank the downstream genetic locus. One amplification product will comprise the first locus. Another amplification product will comprise the second locus. If primers from each pair of primers overlap, an additional amplification product can comprise both the first and second locus. For example, if the reverse primer flanking the upstream locus overlaps (e.g., comprises a sequence capable of annealing in a sequence-specific manner to) the forward primer flanking the downstream locus, an amplification product can comprise both the first and second genetic loci. However, the spacing region between the loci can now be different from the corresponding spacing region on the template. The segments comprising the loci and the spacing region can be short enough to allow phasing, e.g., by sequencing or any other known technique. As another example, if the forward primer flanking the upstream locus overlaps the reverse primer flanking the downstream locus, an amplification product can comprise both loci, though the arrangement would not correspond to that of the template, as the first locus would now be downstream of the second locus. In some embodiments, such an arrangement can be desirable.

In another embodiment, amplification can be performed of the first and second locus using primers which do not overlap, thus producing an amplification product comprising the first locus and an amplification product comprising the second locus; further primers can then be introduced in subsequent amplification to produce a nucleic acid polymer which comprises the first and second locus.

### Nucleic Acid Polymer

In some embodiments, a nucleic acid polymer is a nucleic acid, the base sequence of which comprises the base sequence of portions of a nucleic acid template comprising two or more allelic variants of genetic loci of interest. In some embodiments, a nucleic acid polymer is generated, e.g., by in vitro amplification of two or more portions of the nucleic acid template, wherein each of the amplified portions comprises an allelic variant of a genetic locus of interest. In the nucleic acid template, there may exist spacer region(s) between the genetic loci of interest. In some embodiments, the portions of the nucleic acid template comprising the genetic loci are amplified using in vitro amplification using a pair of primers for each locus (e.g., a forward and a reverse primer flanking a particular genetic locus). In some embodiments, a nucleic acid polymer is produced from a nucleic acid template which comprises a first and second genetic loci, but the spacing region between the loci is altered (e.g., shortened), thus allowing the phasing of the genetic loci. In some embodiments, a nucleic acid polymer is produced from a nucleic acid template via a method comprising a step of amplification using multiple pairs of primers, wherein a pair of primers flanks a first genetic locus, and another pair of primers flanks another genetic locus, and wherein one primer of a pair of primers is capable of annealing in a sequence-specific manner to a primer of another pair of primers, and wherein the nucleic acid polymer is produced from a nucleic acid template which comprises a first and second genetic loci, but the spacing region between the loci is altered from that in the nucleic acid template (e.g., shortened), thus allowing the phasing of the genetic loci.

In some embodiments, the present disclosure provides a method of generating nucleic acid polymers comprising a first and a second genetic locus, the method comprising steps of:

(a) providing a sample comprising one or more types of the nucleic acid template comprising the first and the second genetic locus and a first spacing region between the first and the second genetic locus;

(b) partitioning the sample into aliquots so that a plurality of the aliquots contain no more than one type of the template; and

(c) generating, from each aliquot containing one type of the template, a plurality of nucleic acid polymers based on the template, wherein at least one of the nucleic acid polymers comprises the first and the second genetic locus and a second spacing region that is different from the first spacing region.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

In some embodiments, the present disclosure provides a method of generating a plurality of nucleic acid polymers, wherein at least one of the polymers comprises a first and a second genetic locus of a nucleic acid template comprising the first and the second genetic locus and a first spacing region between the first and the second genetic locus, the method comprising a step of:

generating from a first aliquot of the nucleic acid template a plurality of nucleic acid polymers based on the template, wherein at least one of the nucleic acid polymers comprises the first and the second genetic locus and a second spacing region that is different from the first spacing region; and

wherein:

the first aliquot contains no more than one type of the template; and

at least one second aliquot of the nucleic acid template comprises a different type of the nucleic acid template than the first aliquot.

In some embodiments, each of the first and second genetic loci are any of two or more allelic variants, and the method further comprises the step of phasing the allelic variants of the first and the second genetic locus on the at least one nucleic acid polymer to phase the allelic variants of the first and the second genetic locus on the nucleic acid template.

A nucleic acid polymer is useful for many purposes, including phasing.

A nucleic acid polymer is useful for other purposes, including various scientific investigations. As a non-limiting example, if two genetic loci of interest are very distant from each other on a chromosome or nucleic acid template, it may be technically difficult to study both simultaneously. For example, if both loci involve protein binding, it may be difficult to perform footprinting assays or gel retardation assays, which involve determinations of protein binding, if the loci are very distant from each other. If a nucleic acid polymer retains the key sequences of both loci but bring them closer together, the nucleic acid polymer is a useful tool for genetic analysis and manipulation.

In some embodiments, the sequence of a nucleic acid polymer may be determined, and that information used to generate another nucleic acid polymer, which may retain the base sequence but may have a different chemistry.

In various embodiments, a nucleic acid polymer comprises any one or more of: a nucleotide, a modified nucleotide, and/or a nucleotide analog (e.g., an analog of RNA or DNA). A modified nucleotide or nucleotide analog can differ from a nucleotide in having a modification at the sugar, base and/or internucleotidic linkage.

Analogs of RNA and DNA (e.g., nucleotide analogs) include, but are not limited to: Morpholino, PNA, LNA, BNA, TNA, GNA, ANA, FANA, CeNa, HNA, cEt, tc-DNA, XNA, and UNA. Modified nucleotides include those which are modified in the phosphate, sugar, and/or base. Such modifications include sugar modifications at the 2′ carbon, such as 2′-MOE, 2′-OMe, and 2′-F. A nucleic acid polymer, or a nucleic acid which comprises the same base sequence as the nucleic acid polymer, can comprise any nucleotide, modified nucleotide, and/or nucleotide analog described herein or known in the art.

A nucleic acid polymer can comprise an artificial nucleic acid including PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those known in the art or described herein. A nucleic acid polymer can comprise a modified internucleotidic linkage, such as any of those listed in Table 2 or known in the art.

A nucleic acid polymer has many purposes. In some embodiments, because a nucleic acid polymer retains the arrangement of allelic variants of genetic loci found in a chromosome or other nucleic acid template, the determination of the phasing the allelic variants in the nucleic acid will determine the phasing of the allelic variants on the chromosome or other nucleic acid template.

### Phasing the Genetic Loci on Nucleic Acid Polymers

The present disclosure encompasses methods for phasing allelic variants of genetic loci on a nucleic acid template in a biological sample; in some embodiments, the methods comprise the step of generating nucleic acid polymers from the nucleic acid template. The present disclosure also encompasses compositions and methods pertaining to nucleic acids comprising two or more genetic loci. These nucleic acids are useful for many purposes, including, but not limited to, phasing. Phasing of allelic variants in a nucleic acid polymer also serves to phase the allelic variants on the nucleic acid template.

In some embodiments, phasing of allelic variants in a nucleic acid polymer can be performed using any method known in the art.

In some embodiments, phasing of allelic variants comprises the step of determining the sequence of each of two or more genetic loci.

Any method of determining the sequence of a genetic locus of interest known in the art can used.

In some embodiments, a nucleic acid polymer comprises a first and a second genetic locus, with a spacing region interposed between the loci. In some embodiments, the spacing region is short enough to allow sequencing to be performed of the nucleic acid polymer, thus directly determining the sequences of the first and second genetic loci.

In some embodiments, some nucleic acid polymers comprising an allele of a genetic loci are susceptible to cleavage by a restriction endonuclease, while others are not. As a non-limiting example, some alleles of genetic variants can be distinguished from other variants because some alleles create or destroy a recognition and/or cleavage site for a restriction endonuclease.

In some embodiments, determination of the sequence of genetic locus can be performed by hybridization with a probe. In some embodiments, a probe is a short oligonucleotide or other nucleic acid which can hybridize, until particular experimental conditions, to a nucleic acid comprising one allele of interest but not another. In some embodiments, a probe can be conjugated to a fluorescent marker, such that fluorescence or lack of fluorescence indicates the sequence of the allelic variant.

In some embodiments, an allelic variant can create or destroy a binding site for a protein. Thus, in some embodiments, the sequence of an allelic variant can be determined by any method to qualify or quantity binding of a protein to a binding site, such as a footprinting assay or gel retardation assay.

In some embodiments, it may be sufficient to phase only one nucleic acid template, as this information can be sufficient to determine the arrangement of the allelic variants of interest on both sister chromosomes. For example, at the time of phasing of the nucleic acid polymer, it may already be known what allelic variants a particular subject has; the phasing of a single nucleic acid polymer can thus be sufficient to phase the allelic variants on both sister chromosomes. As a non-limiting example, genetic material can be obtained from a subject suspected of having a disease; this genetic material can be sequenced, revealing that the subject has allelic variants at various loci, though the sequencing data may not reveal the phasing of the allelic variants. For example, a subject may be thus known to have allelic variants of A and a and B and b at two genetic loci, though the arrangement on the sister chromosomes is not known. In this example case, if genetic material from the same subject is then partitioned into aliquots, and nucleic acid polymers produced from it, it may be sufficient to phase only one nucleic acid polymer to determine the phasing on both sister chromosomes.

In some embodiments, phasing of the allelic variants on a nucleic acid polymer can comprise the step of amplification using allele-specific primers. As a non-limiting example, a polymer can comprise a first locus which can have allelic variants of A and a, and a second locus which can have allelic variants of B and b. Phasing of the allelic variants can comprise the step of amplification in the presence of primers capable of differentiating between allelic variants (e.g., primers which anneal to a nucleic acid with the A allelic variant but not a, or B but not b). In some embodiments, a sequence variation (A or a) at the 5′ end of a primer can be particularly effective in differentiating between two alleles (such as A or a). As a non-limiting example, the determination of the allelic variant at a first locus, which can be A or a, can be performed using a forward primer which recognizes A but not a, and a reverse primer. If the locus is A, an amplification product will be produced from the two primers; if the locus is a, no amplification product will be produced. As a non-limiting example, the determination of the allelic variant at a first locus, which can be A or a, can be performed using a forward primer upstream of the locus, a reverse primer which recognizes A but not a, and another reverse primer downstream of the locus, which recognizes the polymer whether the locus is A or a. If the primers are spaced apart, for example, but 50 nucleotides, amplification in the presence of the primers will reveal which allelic variant exists at the locus. For example, a control amplification product of 100 nt will be produced from the forward primer and the downstream reverse primer, whether or not the locus is A or a. However, if the locus is A, a second amplication product will be produced from the forward primer and the reverse primer which recognizes A. If the locus is a, a reverse primer recognizing A will not anneal to the locus, and no second band will be produced. Similarly, in other non-limiting examples, different arrangements of multiple forward primers, multiple reverse primers, and overlapping primers which recognize various allelic variants (or fail to recognize particular allelic variants) can be used to phase the allelic variants. In some embodiments, a method of phasing the allelic variants on a nucleic acid polymer comprises a step of using an ARMS assay or a variant thereof.

In some embodiments, a method of phasing the allelic variants on a nucleic acid polymer can comprise any method of phasing described herein known in the art. Phasing of the nucleic acid polymer will indicate the arrangement of allelic variants on the nucleic acid template, such as a chromosome, chromosome fragment, mRNA, mRNA fragment or cDNA of a biological sample from a subject such as a human.

In some embodiments, phasing of the allelic variants on the nucleic acid polymer is performed by sequencing.

In some embodiments, phasing of the allelic variants on the nucleic acid polymer is performed by next generation sequencing.

In some embodiments, the amplicons produced in the various aliquots are combined and the amplicons are sequenced by next generation sequencing.

In some embodiments, the step of sequencing encompasses any method known in the art of determining the order of the bases or nucleotides in a nucleic acid.

Various methods of sequencing include, for example, Maxam-Gilbert sequencing, Sanger sequencing, sequencing involving radiolabeling, non-radioactive sequencing, sequencing using dideoxy bases, base-by-base sequencing, pyrosequencing, parallelized pyrosequencing, 454 pyrosequencing, sequencing involving capillary electrophoresis, sequencing involving fluorescent labeling, large scale sequencing, shotgun sequencing, DNA colony sequencing, massively parallel signature sequencing, sequencing involving PCR, sequencing involving emulsion PCR, sequencing involving bridge PCR, Polony sequencing, de novo sequencing, single-molecule sequencing, single-molecule real-time sequencing, sequencing by synthesis, sequencing by ligation, sequencing involving an ion semiconductor, massively parallel signature sequencing, DNA nanoball sequencing, sequencing by hybridization, sequencing with mass spectrometry, microfluidic Sanger sequencing, microscopy-based sequencing, sequencing involving RNA polymerase, next generation sequencing, and various other methods known in the art. Various sequencing techniques are described in, for example: Sanger et al. 1975 J. Mol. Biol. 94: 441-8; Sanger et al. 1977 Proc. Natl. Acad. Sci. USA 74: 5463-7; Maxam et al. 1977 Proc. Natl. Acad. Sci. USA 74: 560-4; Smith et al. 1986 Natur. 321: 6745; Prober et al. 1987 Science 238: 336-341; Adams et al. 1991 Science 252: 1651-6; Olsvik et al. 1993 J. Clin. Microbiol. 31: 22-25; Ronaghi et al. 1996 Anal. Biochem. 242: 84-9; Ewing et al. 1998 Genome Res. 8: 186-94; Brenner et al. 2000 Nature Biotech. 18: 630-634; Lander et al. 2001 Nature 409: 860921; Venter et al. 2001 Science 291: 1304-51; Margulies et al. 2005 Nature 437: 376-80; Shendure et al. 2005 Science 309: 1728-32; Peisajovich et al. 2006 Nature Methods 3: 545-550; Stein 2008 Genet. Eng. Biotech. News 28; Schuster 2008 Natur. Methods 5: 16-18; Ten Bosch et al. 2008 J. Mol. Diagnost. 10: 484-492; Pettersson et al. 2009 Genomics 93: 105-11; Tucker et al. 2009 Am. J. Human Genet. 85: 142-154; Rasko et al. 2011 N. Eng. J. Med. 365: 709-717; Quail et al. 2012 BMC Genomics 12: 341; Liu et al. 2012 J. Biomed. Biotech. 2012: 1-11; Chin et al. 2012 Nature Methods 10: 563-9; Quail et al. 2012 Electrophoresis 33: 3521-8; Duhaime et al. 2012 Environ. Microbiol. 14: 1526-37; Peterson et al. 2012 PLoS ONE 7: e37135; Tran et al. 2012 Int. J. Cancer 132: 1547-1555; Murray et al. 2012 Nucl. Acids Res. 20: 11450-62; and Huang et al. 2012 BMC Systems Biol. 6 Supp. 1: S10.

In various embodiments, a nucleic acid polymer is derived from one chromosome or other nucleic acid template; the allelic variants of genetic loci on the nucleic acid polymer thus reflect the arrangement (e.g., sequences) of the allelic variants of the same genetic loci on the chromosome. Thus, using sequencing or some other method to determine the arrangement (e.g., sequences) of the genetic loci on the nucleic acid polymer simultaneously determines the arrangement of the genetic loci on the chromosome.

If the genetic loci are already known to be heterozygous, determining the arrangement of the genetic loci on one sister chromosome also simultaneously determines the arrangement of the genetic loci on the other chromosome. This information is useful for various purposes, including phasing and various scientific analyses.

### Uses

The determination of the arrangement of linkages of allelic variants on an individual's chromosomes is very useful for a number of different reasons. Such a determination, or phasing of the allelic variants, is useful for genetic analysis and for fully exploiting the potential of techniques such as genome engineering and allele-specific expression analysis. It is also useful for patient stratification for allele-specific therapeutics.

In some embodiments, an allele-specific therapeutic is a nucleic acid.

In some embodiments, an allele-specific therapeutic is a nucleic acid which is a RNAi agent, an antisense oligonucleotide, a miRNA or a CRISPR guide nucleic acid (e.g., a guide RNA).

In some embodiments, an allele-specific therapeutic is a therapeutic whose design takes into account allelic variants; in some embodiments, an allele-specific therapeutic can treat one allele of a gene (e.g., a mutant allele), while having a lesser or no effect on another allele (e.g., a wild-type allele). For example, in some diseases, an individual may have a mutant form of one gene, which, being translated, produces a mutant protein (e.g., one which is truncated, or which has a loss-of-function or gain-of-function mutation) on one sister chromosome, while the same individual has a wild-type protein on the other chromosome. If the wild-type protein is a necessary protein, a therapeutic which indiscriminately reduces the level of both the mutant and wild-type proteins may be inappropriate.

In some embodiments, a therapeutic is allele-specific in that it preferentially reduces the level, activity or production of a mutant protein compared to that of the wild-type protein.

In some embodiments, it may not be technically possible or desirable for the allele-specific therapeutic to attack the mutant allele at the site of the deleterious mutation. As non-limiting examples, due to the complex, three-dimensional and partially double-stranded nature of a mRNA, it may not be possible for a RNAi agent to attack a particular site in the mRNA. As an alternative, the allele-specific therapeutic can attack a second site which is present on the mutant allele (but not on the wild-type allele), but which is not the site of the deleterious mutation. For example, many genes are known to have SNPs, or single-nucleotide polymorphisms, wherein at various sites, one allele will differ from another, though the SNPs themselves may not be disease-related. Thus, if a gene is known to have a deleterious mutation on one sister chromosome and not the other; and if the gene is also known to be heterozygous at one or more SNPs, and if phasing determines that a particular SNP (e.g., a C nucleotide at a particular location) is present on the same allele as the deleterious mutation (but not on the wild-type chromosome), then an allele-specific therapeutic can be devised which attacks the SNP with a C, and thus attacks the allele with the deleterious mutation.

In some embodiments, techniques for phasing can be used for patient stratification.

As a non-limiting example, an example patient may have a particular disease linked to a particular mutation. In some embodiments, the patient may be under consideration for treatment with an allele-specific therapeutic which attacks a particular allelic variant of a genetic locus. In some embodiments, that locus is a SNP. In some embodiments, if phasing reveals that the same chromosome of the patient carries both the deleterious mutation and the particular allele recognized by the allele-specific therapeutic, the allele-specific therapeutic can be used to treat the patient.

In a non-limiting example, an example patient may have Huntington's Disease, which is linked to a mutation in the Huntingtin gene (HTT) comprising an excessive number of repeats (e.g., a repeat expansion) of the sequence CAG. In some embodiments, the patient may be under consideration for treatment with an allele-specific therapeutic (e.g., an antisense oligonucleotide or RNAi agent) which recognizes a particular allelic variant of a genetic locus in the HTT gene (which is outside the repeat expansion), as a non-limiting example, a SNP. If phasing reveals that the the same chromosome of the patient comprises both the repeat expansion and the particular allelic variant of a genetic locus (e.g., a SNP) recognized by the allele-specific therapeutic, then the patient is eligible for treatment with the allele-specific therapeutic.

Phasing can thus be used to determine the various SNPs that a particular individual has in relation to a particular mutant or wild-type allele of a gene. For example, U.S. Patent Application Nos. 62/195,779, filed Jul. 22, 2015, 62/331,960, filed May 4, 2016, and WO2015107425, describe various antisense oligonucleotides which recognize particular alleles of the Huntingtin gene, wherein mutant forms of the gene are involved in Huntington's Disease. Phasing will allow the determination of whether particular SNPs in the Huntingtin gene are on the same chromosome as the mutant allele; if so, then an antisense oligonucleotide or RNAi agent which recognizes this SNP can be used to attack the mutant allele.

The determination of the phase of various allelic variants of genetic loci is thus useful for a large number or purposes.

In some embodiments, the present disclosure provides methods for treating a condition in a subject, comprising administering to a subject a pharmaceutical composition comprising one or more reagents targeting a first genetic locus, wherein the first genetic locus is characterized in that a transcript from the first genetic locus is within a single transcript with that of a second genetic locus which is associated with the condition by a provided phasing method. In some embodiments, a condition is a disease. In some embodiments, a condition is cancer. In some embodiments, a condition is Huntington's disease. In some embodiments, a subject is a patient. In some embodiments, a reagent targeting a first genetic locus is an oligonucleotide. In some embodiments, a pharmaceutical composition comprises a chirally controlled oligonucleotide composition. In some embodiments, a first genetic locus comprises a characteristic sequence element. In some embodiments, a first genetic locus comprises a SNP. In some embodiments, a first genetic locus comprises a SNP in Huntingtin, and is at least 20, 30, 50, or 100 bp in length. In some embodiments, a second genetic locus comprises a characteristic sequence element. In some embodiments, a second genetic locus comprises expanded CAG repeats associated with Huntington's disease. In some embodiments, transcript of the SNP and the CAG repeats are within the same mRNA. Example characteristic sequence elements (e.g., SNP, repeats, etc.), oligonucleotide compositions, methods, etc., include those described in PCT/US16/43542, which is incorporated herein by reference.

### EXAMPLES

The foregoing has been a description of certain non-limiting embodiments of the disclosure. Accordingly, it is to be understood that the embodiments of the disclosure herein described are merely illustrative of the application of the principles of the disclosure. Reference herein to details of the illustrated embodiments is not intended to limit the scope of the claims.

### Example 1: ARMS (Amplification-Refractory Mutation System) ASSAY

Huntington's disease (HD) is a genetic autosomal-dominant neurodegenerative disorder caused by a poly-glutamine expansion in huntingtin, the protein encoded by the HD gene. Any effective molecular therapy must preserve the expression of wild-type huntingtin, while silencing the mutant allele. Allele specific oligonucleotide approaches, based on antisense oligonucleotides, provide promising new therapeutic strategies for direct intervention through reduced production of the causative mutant protein. Heterozygous polymorphisms in cis with the mutation allow for allele-specific suppression of the pathogenic HTT transcript. To include patients for allele specific therapy, precise heterozygosity analysis is needed. Here we describe a PCR based method for analyzing the heterozygosity of SNPs rs362307, rs362331 and rs2530595, located in the genomic region of HD gene and known to be heterozygous for a major portion of HD population.

### Arms Assay:

The ARMS assay (also known as allele-specific PCR) is a low cost and simple method for detecting any mutation, as non-limiting examples, those involving single base changes or small deletions. The assay is based on the use of sequence-specific PCR primers that allow amplification of test DNA only when the target allele is contained within the sample. It uses four primers in a single Polymerase Chain Reaction (PCR) and is followed just by gel-based resolution of tetra-primer PCR products. The basis of the assay is that introducing a mismatch at the 3′-end of the primers will not work in the PCR under appropriate optimized conditions. Taq DNA polymerases are extremely effective at distinguishing between a match and a mismatch at the 3′-end of a PCR primer. When the primer is fully matched, the amplification proceeds with full efficiency. The primers are also designed such that the two primer pairs overlap at a SNP location but each match perfectly to only one of the possible SNPs and their PCR products are of a significantly different length allowing for easily distinguishable bands by gel electrophoresis. If a SNP is homozygous, then the PCR products that result can be from the primer that matches the SNP location and the outer opposite-strand primer, as well from the two outer primers. If the SNP is heterozygous, then products will result from the primer of each allele and their respective outer primer counterparts as well as the outer primers.

The protocols detailed here outline three ARMS assay methods that can be used for genotype analysis of the SNPs rs362307, rs362331 and rs2530595, all these polymorphisms are located in the genomic region of Huntington gene. Example primer information and assay conditions were shown in the figures.

### REFERENCES

Multiplex genotyping assay for detecting mutations in k-ras
WO 2010048691 A1
Cystic fibrosis test based on the detection of mutations in the CFRE gene by ARMS
EP 0928832 A2
Method for detecting variant nucleotides using arms multiplex amplification
EP 1151136 A1
ARMS-PCR method for mtDNA allelic gene typing and point mutation detecting
CN 101768635 B
ARMS fluorescent quantitative PCR-based gene mutation kit and method thereof
CN 104031978 A
Kit for detecting 858 codon mutation of EGFR (Epidermal Growth Factor Receptor) gene and application thereof
CN 103882137 A

### Example 2 Droplet Phasing

This example provides various example steps in droplet phasing.

Droplet Phasing by cloning:
1. Template preparation:
   a. Genomic DNA
      The genomic DNA was prepared by any methods known to the field. For example, MegaLong™ For Isolation of >100 kb Genomic DNA (G-Biosciences), or QIAamp® DSP DNA Mini Kit (QIAGEN), according to manufacturer's protocol. Final DNA concentration is determined by NanoDrop ND-2000 (ThermoFisher)
   b. cDNA
      Total RNA was purified by any methods known to the field. For example, RNeasy Mini Kit (QIAGEN) or TurboCapture mRNA kit (QIAGEN) or TRIzol® Reagent (Thermo Fisher Scientific).
      cDNA was synthesized by any methods known to field. For example, Transcriptor First Strand cDNA Synthesis Kit (Roche) or SuperScript IV Reverse Transcriptase (ThermoFisher)
2. Droplet generation:
   a. PCR mix
      i. Primers: See table 1 for sequences used for the figures (rs362307). Table 2 contains all the primers to phase additional rs2530595, rs362331, rs7685686

TABLE 1
Example Primers
Primers for Droplet primary PCR

| | |
|---|---|
| CAG-F1 | GGCGACCCTGGAAAAGCTG |
| CAG-F4 | ATGGCGACCCTGGAAAAGCTGAT |
| CAG-R2-23GAP-R | CACTGTCATGCCGTTACGTAGCGGACAATGATTCACACGGTCT |
| CAG-R3-23GAP-R | CACTGTCATGCCGTTACGTAGCGTGAGGCAGCAGCGGCTGT |
| H307SNP-F2-23GAP-F | CGCTACGTAACGGCATGACAGTGCGAAATGTCCACAAGGTCACCACCTG |
| H307SNP-F3-23GAP-F | CGCTACGTAACGGCATGACAGTGGCCATGGTGGGAGAGACTGTGAGG |
| H307SNP-R1 | ATAGGGACCAAGCTGGCTCGGTGGAG |
| H307SNP-R2 | ATGTGCGGAAGCCCATAGGGACCAAG |

Primers to amplify Long Amplicon

| | |
|---|---|
| CAG-F1-Link | 5′ TCGTCGGCAGCGTCAGATGTGTATAAGAGACAGGGCGACCCTGGAAAAGCTG |
| H307SNP-R1-Link | 5′ GTCTCGTGGGCTCGGAGATGTGTATAAGAGACAGATAGGGACCAAGCTGGCT |
| | CGGTGGAG |
| H307SNP-R2-Link | 5′ GTCTCGTGGGCTCGGAGATGTGTATAAGAGACAGATGTGCGGAAGCCCATAG |
| | GGACCAAG |

TABLE 2
Primers to phase additional rs2530595, rs362331, rs7685686

| | |
|---|---|
| Name | Sequence |

Primers for Droplet primary PCR (23 nt GAP)

| | |
|---|---|
| CAG-F1 | GGCGACCCTGGAAAAGCTG |
| CAG-F4 | ATGGCGACCCTGGAAAAGCTGAT |
| CAG-R2-23GAP-R | CACTGTCATGCCGTTACGTAGCGGACAATGATTCACACGGTCT |
| CAG-R3-23GAP-R | CACTGTCATGCCGTTACGTAGCGTGAGGCAGCAGCGGCTGT |
| H307SNP-F3-23GAP | CGCTACGTAACGGCATGACAGTG-GCCATGGTGGGAGAGACTGTGAGG |
| H331SNP-F3-23GAP | CGCTACGTAACGGCATGACAGTG-GGAGCGTGGTCTCCTCCACAGAGTT |
| H595SNP-F3-23GAP | CGCTACGTAACGGCATGACAGTG-AGCAGACATCCTCATCGGGCTTTGT |
| H686SNP-F1-23GAP | CGCTACGTAACGGCATGACAGTG-AGCCGACTCTCCAACTGAAAGAGGTG |
| H331SNP-R1 | TCGCTGATGGCTTTTGGGGTATTTG |
| H331SNP-R2 | AAGAAGCTGCTCTCCAGGCTGCACT |
| H331SNP-R3 | GCCTCCAGGATGAAGTGCACACAGT |
| H595SNP-R2 | CCTACACGTGACCCCTCTGGAGGAC |
| H595SNP-R1 | TCCTGAGCTCTCCAAGGTCCCTCAG |
| H686SNP-R2 | GCTTCAGAATTTGGAGCATTTTGGA |
| H686SNP-R1 | AAGCTTCAGAATTTGGAGCATTTTGGA |

Primers for Droplet primary PCR (41 nt GAP)

| | |
|---|---|
| CAG-R2-41GAP-R | CTACTCCTTCAGTCCATGTCAGTGTCCTCGTGCTCCAGTCGGACAATGATTCACAC |
| | GGTCT |
| CAG-R3-41GAP-R | CTACTCCTTCAGTCCATGTCAGTGTCCTCGTGCTCCAGTCGTGAGGCAGCAGCG |
| | GCTGT |
| H307SNP-F3-41GAP | CGACTGGAGCACGAGGACACTGACATGGACTGAAGGAGTAGGCCATGGTGGGA |
| | GAGACTGTGAGG |
| H595SNP-F3-41GAP | CGACTGGAGCACGAGGACACTGACATGGACTGAAGGAGTAGAGCAGACATCCT |
| | CATCGGGCTTTGT |
| | CGACTGGAGCACGAGGACACTGACATGGACTGAAGGAGTAGGGAGCGTGGTCT |
| H331SNP F3-41GAP | CCTCCACAGAGTT |
| H686SNP-F1-41GAP | CGACTGGAGCACGAGGACACTGACATGGACTGAAGGAGTAGAGCCGACTCTCCAAC |
| | TGAAAGAGGTG |

Make Droplet PCR reagent according to following table 3,

TABLE 3
Recipe for ddPCR reaction mixture (rs362307 example)

| | rs362307 | Final conc. | | Stock | | ul |
|---|---|---|---|---|---|---|
| Genomic | | 20 | ng | 80 | ng/ul | 0.5 |
| CAG-F1 | CAG-F1-Link | 500 | nM | 100 | uM | 1 |
| CAG-RxG | CAG-R3-41GAP-R | 60 | nM | 20 | uM | 0.6 |
| SNP-FxG | 307SNP-F3-41GAP | 60 | nM | 20 | uM | 0.6 |
| SNP-Rx | 307SNP-R2 | 500 | nM | 100 | uM | 1 |

| | | | |
|---|---|---|---|
| 2 x ddPCR | 1 x ddPCR | 2 x | 10 |
| reagent | | | |
| Distilled | | | 6.3 |
| water | | | |
| Total | | | 20 |

b. Droplet generation
   i. Droplet generation
      1. Setup droplet generation according to manufacturer's protocol (BioRad QX200 Droplet Generator).
      2. Samples are duplicates. One of duplicates contains probes for gene of interest, in order to do digital PCR on QX200 Droplet Reader. This is to exam the proper dilution of template. The other duplicate is for DNA extraction.
3. PCR and DNA recovery
   a. After droplet generate do the PCR on regular thermocycler, for example, C1000 Touch™ Thermal Cycler with following protocol: 94 C, 3 min, (94 C, 20 sec; 60 C, 45 sec; 72 C, 90 sec)×40 cycles, 72 C, 5 min, 4 C forever.
   b. After droplet PCR, add chloroform to breakup Droplet. Add 20 ul of TE, extract DNA with Phenol/Chloroform, and precipitate DNA with 0.3 M sodium acetate pH5.2, and 2.5 volumes of cold 100% ethanol.
4. 2^{nd} PCR to enrich “long fragment” and add NGS primer linker. Table 4 lists all the primers to phase rs362307, rs2530595, rs362331, rs7685686 in this step.

TABLE 4
Primers to amplify phased Long Amplicons
Primers to amplify Long Amplicon

| | |
|---|---|
| CAG-F1-Link | TCGTCGGCAGCGTCAGATGTGTATAAGAGACAG-GGCGACCCTGGAAAAGCTG |
| H307SNP-R1-Link | GTCTCGTGGGCTCGGAGATGTGTATAAGAGACAG-ATAGGGACCAAGCTGGCTCGGTGGAG |
| H307SNP-R2-Link | GTCTCGTGGGCTCGGAGATGTGTATAAGAGACAG-ATGTGCGGAAGCCCATAGGGACCAAG |
| H595SNP-R2-link | GTCTCGTGGGCTCGGAGATGTGTATAAGAGACAG-CCTACACGTGACCCCTCTGGAGGAC |
| H595SNP-R1-link | GTCTCGTGGGCTCGGAGATGTGTATAAGAGACAG-TCCTGAGCTCTCCAAGGTCCCTCAG |
| H331SNP-R1-link | GTCTCGTGGGCTCGGAGATGTGTATAAGAGACAG-TCGCTGATGGCTTTTGGGGTATTTG |
| H331SNP-R2-link | GTCTCGTGGGCTCGGAGATGTGTATAAGAGACAG-AAGAAGCTGCTCTCCAGGCTGCACT |
| H331SNP-R3-link | GTCTCGTGGGCTCGGAGATGTGTATAAGAGACAG-GCCTCCAGGATGAAGTGCACACAGT |
| H686SNP-R1-link | GTCTCGTGGGCTCGGAGATGTGTATAAGAGACAG-AAGCTTCAGAATTTGGAGCATTTTGGA |
| H686SNP-R2-link | GTCTCGTGGGCTCGGAGATGTGTATAAGAGACAG-GCTTCAGAATTTGGAGCATTTTGGA |

TABLE 5
Recipe for amplification of “long” fragment
reaction mixture (rs362307 example)

| | rs362307 | Final conc. | Stock | ul |
|---|---|---|---|---|
| Extracted ddPCR | | | | 1 |
| frag | | | | |
| CAG-F1-Link | CAG-F1-Link | 300 nM | 10 uM | 0.3 |
| H307SNP-R1-Link | H307SNP-R1-Link | 300 nM | 10 uM | 0.3 |
| 2 x PCR mix | | 1 x PCR mix | 2 x | 10 |
| Distilled water | | | | 8.4 |
| Total | | | | 20 |

Note: 2×PCR mix can be from any company, for example: iQ Powermix (BioRad); KAPA HiFi ReadyMix (HAPA); TaqMan Universal PCR Master Mix (LifeTechnologies); MyTaq Mix (Bioline); LightCycler TaqMan Master (Roche).
PCR: C1000 Touch™ Thermal Cycler with following protocol: 94 C, 3 min, (94 C, 20 sec; 60 C, 45 sec; 72 C, 90 sec)×25 cycles, 72 C, 5 min, 4 C forever.
Fragment analysis (AATI-us.com) or 2100 Bioanalyzer (Agilent.com) to confirm correct PCR fragment generation.
5. Bar-code, QC, multiplex. The bar-code was done by using Nextera XT Sample Preparation Kit with bar-code kit (FC-131-2001, Illumina).

TABLE 6
Recipe for ddPCR reaction mixture (rs362307 example)

| | | ul |
|---|---|---|
| Amplified “long” frag | | 0.3 |
| N5xx | Bar-code primers | 2 |
| N7xx | Bar-code primers | 2 |
| 2 x PCR mix | | 5 |
| Distilled water | | 0.7 |
| Total | | 10 |

Note: 2×PCR mix can be from any company, for example: iQ Powermix (BioRad);

KAPA HiFi ReadyMix (HAPA); TaqMan Universal PCR Master Mix

(LifeTechnologies); MyTaq Mix (Bioline); LightCycler TaqMan Master (Roche).

Bar-coded PCR fragments (Libraries) were purified by AMPure XP beads (Agencourt) according to manufacturer's protocol.

Each sample (library) is QC'd and quantified by Fragment analysis (AATI) or 2100 Bioanalyzer (Agilent), equal molar amount of samples are combined. A final library QC was done again before submitting the samples for MiSeq run
6. MiSeq run is done on Illumina's MiSeq using 2×150 paired-end run according to manufacturer's protocol. In some cases, PhiX DNA (10-30%) was spike-in to increase library diversity, in order to be successfully run on MiSeq.
7. Bioinformatics analysis is done by custom program with following workflow: 1) assemble amplicon from both end's reads. P7/i7 read would be R1 which is read from 5′ end of amplicon. It covers maximum of 34×CAG (or 84×CAG if run is 2×300 PE). P5/i5 read would be R2 which is read from 5′ end of amplicon, which covers SNPs. There might be gap between the assembled amplicon. 2) Count the number of reads of each SNP and its association of normal or expanded CAG, to get final phasing results.

### Example 3 Example of Phasing Procedure

To extract high molecule weight genomic DNA, dialysis based genomic DNA extraction kits were, such as MegaLong™ (G-Biosciences) or similar kits. Patient derived fibroblast cultured cells or patient primary lymphocytes (primary blood mononuclear cells, PBMC) were harvested by centrifugation, washed with Phosphate Buffered Saline (PBS), followed by the MegaLong™ protocol. MegaLong™ isolates nuclei under mild extraction conditions in Nuclei Isolation Buffer and releases genomic DNA by digestion of nuclear proteins with a highly active LongLife™ Proteinase K. The digestion was performed in the Tube-O-DIALYZER™ (0.45 um membrane) and after digestion the Tube-O-DIALYZER™ was inverted to dialyze away digested protein and other impurities leaving behind highly pure and fully hydrated genomic DNA. After dialysis at 4 C (usually two days with few dialysis buffer exchange), the high molecular weight DNA were harvested by wide-mouth pipet tips, aliquoted and frozen at −80 C for long term storage, or 4 C for short term storage (<1 month). Avoid frequent freeze and thaw of −80 C stored genomic DNA.

To generate cDNA from patient derived fibroblast cultured cells or patient primary lymphocytes PBMC, cells were harvested by centrifugation, washed with PBS, and followed by the RNeasy Mini kit (QIAGEN). Briefly, cells were lysed in RLT buffer. After adding 1 volume of 70% ethanol, lysate were transferred to an RNeasy Mini spin column, centrifuged for 15 s at ≥8000×g. The columns were washed once with 700 ul of Buffer RW1, twice with 500 ul of Buffer RPE, and finally eluted with 30-50 ul of RNase-Free water. cDNA were synthesized by SuperScript iv reverse transcriptase (Invitrogen). 1-5 ug of total RNA were mixed with 5×SSIV buffer, 0.5 mM dNTP mix, 5 mM DTT, RNaseOUT RNase Inhibitor, 2.5 uM of Oligo d(T)20 primer and Superscript IV Reverse Transcriptase, incubated at 50-55 degrees Celsius for 10 min, inactivated the reaction by incubating it at 80 degrees Celsius for 10 min. The cDNA is ready to use.

To setup Droplet PCR, either genomic DNA or cDNA was used in BioRad QX200 Digital PCR System. Briefly, the following primer were used.

Primers for Droplet primary PCR

| | |
|---|---|
| CAG-F1 | GGCGACCCTGGAAAAGCTG |
| CAG-F4 | ATGGCGACCCTGGAAAAGCTGAT |
| CAG-R2-23GAP-R | CACTGTCATGCCGTTACGTAGCGGACAATGATTCACACGGTCT |
| CAG-R3-23GAP-R | CACTGTCATGCCGTTACGTAGCGTGAGGCAGCAGCGGCTGT |
| H307SNP-F2-23GAP-F | CGCTACGTAACGGCATGACAGTGCGAAATGTCCACAAGGTCACCACCTG |
| H307SNP-F3-23GAP-F | CGCTACGTAACGGCATGACAGTGGCCATGGTGGGAGAGACTGTGAGG |
| H307SNP-R1 | ATAGGGACCAAGCTGGCTCGGTGGAG |
| H307SNP-R2 | ATGTGCGGAAGCCCATAGGGACCAAG |

Primers to amplify Long Amplicon

| | |
|---|---|
| CAG-F1-Link | 5′ TCGTCGGCAGCGTCAGATGTGTATAAGAGACAGGGCGACCCTGGAAAAGCTG |
| H307SNP-R1-Link | 5′ GTCTCGTGGGCTCGGAGATGTGTATAAGAGACAGATAGGGACCAAGCTGGCT |
| | CGGTGGAG |
| H307SNP-R2-Link | 5′ GTCTCGTGGGCTCGGAGATGTGTATAAGAGACAGATGTGCGGAAGCCCATAG |
| | GGACCAAG |

1 ng genomic DNA or 1:100-1:100,000 dilution of cDNA were mixed with CAG-F1 and H307SNP-R2 (500 nM final concentration) plus CAG-R3-23GAP-R and H307SNP-F3-23GAP-F (60 nM final concentration). Note any combination of above primer sets worked. 12.5 ul of DNA templates (genomic or cDNA) with primers were mixed with 12.5 ul of ddPCR Supermixes for Probes reagent (BioRad), then generated droplet according to manufacturer's protocol (BioRad QX200 Droplet Generator). After droplet generation, do the PCR on regular thermocycler, for example, C1000 Touch™ Thermal Cycler with following protocol: 94 C, 3 min, (94 C, 20 sec; 60 C, 45 sec; 72 C, 90 sec)×40 cycles, 72 C, 5 min, 4 C forever.

After droplet PCR, chloroform was added to the PCR mixture to break up the Droplet. 20 ul of TE was added, DNA was extracted with Phenol/Chloroform, and DNA precipitated with 0.3 M sodium acetate pH 5.2, and 2.5 volumes of cold 100% ethanol. Final DNA was dissolved in 20 ul of TE buffer.

For 2nd PCR to enrich “long fragment” and add NGS primer linker, Table 4 lists all the primers to phase SNPs rs362307, rs2530595, rs362331, rs7685686 in this step. Example primers are presented below:

| | |
|---|---|
| Name | Sequence |

Primers to amplify Long Amplicon

| | |
|---|---|
| CAG-F1-Link | TCGTCGGCAGCGTCAGATGTGTATAAGAGACAG-GGCGACCCTGGAAAAGCTG |
| H307SNP-R1-Link | GTCTCGTGGGCTCGGAGATGTGTATAAGAGACAG-ATAGGGACCAAGCTGGCTCGGTGGAG |
| H307SNP-R2-Link | GTCTCGTGGGCTCGGAGATGTGTATAAGAGACAG-ATGTGCGGAAGCCCATAGGGACCAAG |
| H595SNP-R2-link | GTCTCGTGGGCTCGGAGATGTGTATAAGAGACAGCCTACACGTGACCCCTCTGGAGGAC |
| H595SNP-R1-link | GTCTCGTGGGCTCGGAGATGTGTATAAGAGACAGTCCTGAGCTCTCCAAGGTCCCTCAG |
| H331SNP-R1-link | GTCTCGTGGGCTCGGAGATGTGTATAAGAGACAGTCGCTGATGGCTTTTGGGGTATTTG |
| H331SNP-R2-link | GTCTCGTGGGCTCGGAGATGTGTATAAGAGACAGAAGAAGCTGCTCTCCAGGCTGCACT |
| H331SNP-R3-link | GTCTCGTGGGCTCGGAGATGTGTATAAGAGACAGGCCTCCAGGATGAAGTGCACACAGT |
| H686SNP-R1-link | GTCTCGTGGGCTCGGAGATGTGTATAAGAGACAGAAGCTTCAGAATTTGGAGCATTTTGGA |
| H686SNP-R2-link | GTCTCGTGGGCTCGGAGATGTGTATAAGAGACAGGCTTCAGAATTTGGAGCATTTTGGA |

1 ul of PCR products were mixed with CAG-F1-Link and H307-SNP-R2-Link (500 nM final concentration). 12.5 ul of DNA templates with primers were mixed with 12.5 ul of iQ PowerMix (BioRad) or KAPA HiFi ReadyMix (KAPA), then ran PCR with C1000 Touch™ Thermal Cycler with following protocol: 94 C, 3 min, (94 C, 20 sec; 60 C, 45 sec; 72 C, 90 sec)×25 cycles, 72 C, 5 min, 4 C forever. Following PCR, Fragment analysis (AATI-us.com) or 2100 Bioanalyzer (Agilent.com) were used to confirm correct PCR fragment generation.

For Bar-coding fragments used for next generation sequencing (NGS), the bar-code (Nextera XT Sample Preparation Kit, FC-131-2001, Illumina), 2 ul each of N5xx/N7xx combination, 0.3 ul of “long fragment” PCR products from above process were mixed with equal volume of iQ PowerMix (BioRad) or KAPA HiFi ReadyMix (KAPA), then ran PCR with C 1000 Touch™ Thermal Cycler with following protocol: 94 C, 3 min, (94 C, 20 sec; 60 C, 45 sec; 72 C, 90 sec)×25 cycles, 72 C, 5 min, 4 C forever. Following PCR, Fragment analysis (AATI-us.com) or 2100 Bioanalyzer (Agilent.com) were used to confirm correct PCR fragment generation. After confirming the PCR fragment size, Bar-coded PCR fragments (Libraries) were purified by AMPure XP beads (Agencourt) according to manufacturer's protocol. Briefly, 10 ul of PCR reaction were mixed 18 ul of AMPure XP beads, settled on magnetic field and washed twice by 70% ethanol, air-dried and resuspended in 20 ul of TE buffer. Each sample (library) was QC'd and quantified by Fragment analysis (AATI) or 2100 Bioanalyzer (Agilent), equal molar amount of samples were combined. A final library QC was done again before submitting the samples for MiSeq run.

MiSeq (Illumina) run was done at Molecular Biology Core Facility on Illumina's MiSeq using 2×150 paired-end run according to manufacturer's protocol. In some cases, PhiX DNA (10-30%) was spike-in to increase library diversity, in order to be successfully run on MiSeq.

Bioinformatics analysis was done by custom program with following workflow: 1) assemble amplicon from both end's reads. P7/i7 read would be R1 which is read from 5′ end of amplicon. It covers maximum of 34×CAG (or 84×CAG if run is 2×300 PE). P5/i5 read would be R2 which is read from 5′ end of amplicon, which covers SNPs. There might be gap between the assembled amplicon. 2) Count the number of reads of each SNP and its association of normal or expanded CAG, to get final phasing results.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A method of phasing allelic variants of a first and a second genetic locus on a nucleic acid template comprising the first and the second genetic locus and a first spacing region between the first and the second genetic locus, the method comprising steps of:
(a) providing a sample comprising one or more types of the nucleic acid template;
(b) partitioning the sample into aliquots so that a plurality of the aliquots contain no more than one type of the template;
(c) generating, from at least one aliquot containing one type of the template, a plurality of nucleic acid polymers based on the template, wherein at least one of the nucleic acid polymers comprises the first and the second genetic locus and a second spacing region that is different from the first spacing region; and
(d) phasing the first and the second genetic locus on the at least one nucleic acid polymer to phase the allelic variants of the first and second genetic loci on the nucleic acid template.

2. A method of phasing allelic variants of multiple genetic loci on a nucleic acid template comprising the multiple genetic loci and multiple spacing regions between the multiple genetic loci, the method comprising steps of:
(a) providing a sample comprising one or more types of the nucleic acid template;
(b) partitioning the sample into aliquots so that a plurality of the aliquots contain no more than one type of the template;
(c) generating, from at least one aliquot containing one type of the template, a plurality of nucleic acid polymers based on the template, wherein at least one of the nucleic acid polymers comprises the multiple genetic loci and wherein at least one of the multiple spacing regions on the nucleic acid polymer is different from the corresponding spacing region on the nucleic acid template; and
(d) phasing the multiple genetic loci on the at least one nucleic acid polymer to phase the allelic variants of the multiple genetic loci on the nucleic acid template.

3. A method of phasing allelic variants of a first and a second genetic locus on a nucleic acid template comprising the first and the second genetic locus and a first spacing region between the first and the second genetic locus, the method comprising a step of:
(a) phasing the allelic variants on a nucleic acid polymer in order to phase the allelic variants on the nucleic acid template,
wherein the nucleic acid polymer comprises the first and the second genetic locus, and a second spacing region which is different from the first spacing region between the first and the second genetic locus,
wherein the nucleic acid polymer is generated from an aliquot containing one type of the nucleic acid template, and
wherein the aliquot is generated by partitioning a sample comprising one or more types of the nucleic acid template into aliquots so that a plurality of the aliquots contain no more than one type of the template.

4. A method of phasing allelic variants of multiple genetic loci on a nucleic acid template comprising the multiple genetic loci and multiple spacing regions between the multiple genetic loci, the method comprising a step of:
phasing the allelic variants on a nucleic acid polymer in order to phase the allelic variants on the nucleic acid template,
wherein the nucleic acid polymer comprises the multiple genetic loci, and wherein one or more of the multiple spacing regions on the nucleic acid polymer is different than one or more of the multiple spacing regions on the nucleic acid template,
wherein the nucleic acid polymer is generated from an aliquot containing one type of the nucleic acid template, and
wherein the aliquot is generated by partitioning a sample comprising one or more types of the nucleic acid template into aliquots so that a plurality of the aliquots contain no more than one type of the template.

5. A method of generating nucleic acid polymers comprising a first and a second genetic locus, the method comprising steps of:
(a) providing a sample comprising one or more types of the nucleic acid template comprising the first and the second genetic locus and a first spacing region between the first and the second genetic locus;
(b) partitioning the sample into aliquots so that a plurality of the aliquots contain no more than one type of the template; and
(c) generating, from each aliquot containing one type of the template, a plurality of nucleic acid polymers based on the template, wherein at least one of the nucleic acid polymers comprises the first and the second genetic locus and a second spacing region that is different from the first spacing region.

6. The method of claim 5, wherein each of the first and second genetic loci are any of two or more allelic variants, and wherein the method further comprises the step of phasing the allelic variants of the first and the second genetic locus on the at least one nucleic acid polymer to phase the allelic variants of the first and the second genetic locus on the nucleic acid template.

7. A method of phasing allelic variants of a first and a second genetic locus on a nucleic acid template comprising the first and the second genetic locus and a first spacing region between the first and the second genetic locus, the method comprising:
generating from a first aliquot of the nucleic acid template a plurality of nucleic acid polymers based on the template, wherein at least one of the nucleic acid polymers comprises the first and the second genetic locus and a second spacer region that is different from the first linking region; and
wherein at least one second aliquot of the nucleic acid template comprises a different type of the nucleic acid template than the first aliquot.

8. The method of claim 7, wherein the first aliquot of the nucleic acid template contains no more than one type of the nucleic acid template.

9. The method of claim 7 or 8, wherein each of the nucleic acid polymers comprises the first and the second genetic locus and a second spacer region that is different from the first linking region.

10. A method of generating a plurality of nucleic acid polymers, wherein at least one of the polymers comprises a first and a second genetic locus of a nucleic acid template comprising the first and the second genetic locus and a first spacing region between the first and the second genetic locus, the method comprising a step of:
generating from a first aliquot of the nucleic acid template a plurality of nucleic acid polymers based on the template, wherein at least one of the nucleic acid polymers comprises the first and the second genetic locus and a second spacing region that is different from the first spacing region; and wherein:
the first aliquot contains no more than one type of the template; and
at least one second aliquot of the nucleic acid template comprises a different type of the nucleic acid template than the first aliquot.

11. The method of claim 10, wherein each of the first and second genetic loci are any of two or more allelic variants, and wherein the method further comprises the step of phasing the allelic variants of the first and the second genetic locus on the at least one nucleic acid polymer to phase the allelic variants of the first and the second genetic locus on the nucleic acid template.

12. A method comprising steps of:
(a) providing a collection of discrete reaction aliquots, at least one of which contains:
a single molecule of a nucleic acid template whose base sequence includes a first sequence element and a second sequence element, wherein the first and second sequence elements are separated from one another in the template nucleic acid by a spacing sequence element comprising a plurality of residues, and further wherein at least one of the first and/or second sequence elements is present in two or more different forms within the set of template nucleic acids present in the collection; and
reagents for amplifying nucleic acids;
(b) incubating the at least one reaction aliquot under conditions and for a time sufficient to generate, within the at least one reaction aliquot, a product nucleic acid in which the first and second sequences, or the complement sequences thereof, are linked to one another and some or all of the residues of the spacing element, or the complement thereof, are absent.

13. The method of claim 12, the method further comprising the step of determining the forms of each of the first and second sequence element on the product nucleic acid, in order to determine the forms of each of the first and second sequence element on the nucleic acid template.

14. A method comprising:
incubating one or more discrete reaction aliquots of a collection, wherein each reaction aliquot of the collection contains:
a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection; and
reagents for amplifying nucleic acids;
under conditions and for a time sufficient to generate, within each discrete reaction aliquot, a product nucleic acid in which the first and second sequences, or the complement sequences thereof, are linked to one another and some or all of the residues of the spacing element, or the complement thereof, are absent.

15. The method of claim 14, the method further comprising the step of determining the forms of each of the first and second sequence element on the product nucleic acid, in order to determine the forms of each of the first and second sequence element on the nucleic acid template.

16. A composition comprising:
a single molecule of a nucleic acid template whose base sequence includes a first sequence element and a second sequence element, wherein the first and second sequence elements are separated from one another in the template nucleic acid by a spacing sequence element that comprises a plurality of residues;
a plurality of nucleic acid molecules in each of which the first and second sequence elements, or the complement sequences thereof, are linked to one another and some or all of the residues of the spacing sequence element, or the complement thereof, are absent; and
optionally an reagent for amplifying the template nucleic acid,
wherein no molecules other than the nucleic acid template and the plurality of nucleic acid molecules contain both the first and the second sequences.

17. A composition comprising a collection of discrete compositions, each of which independently contains:
a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the first and/or second sequence elements is present in two or more different forms within the set of template nucleic acids present in the collection; and
a plurality of nucleic acid molecules, in each of which the first and second sequences, or the complement sequences thereof, are linked to one another and some or all of the residues of the spacing sequence element, or the complement thereof, are absent; and
optionally an reagent for amplifying the template nucleic acid.

18. A composition comprising a collection of discrete compositions, each of which independently contains:
a single molecule of a template nucleic acid whose base sequence includes a first sequence element and a second sequence element, wherein the first and second sequence elements are separated from one another in the template nucleic acid by a spacing sequence element that comprises a plurality of residues, and further wherein at least one of the sequence elements is present in two or more different forms within the set of template nucleic acids present in the collection;
a plurality of nucleic acid molecules in each of which the first and second sequences, or the complement sequences thereof, are linked to one another and some or all of the residues of the spacing element, or the complement thereof, are absent; and
optionally an reagent for amplifying the template nucleic acid;
wherein in each composition, no molecules other than the template nucleic acid and the plurality of nucleic acid molecules contain both the first and the second sequences.

19. A method comprising:
incubating one or more discrete reaction systems of a collection, wherein each reaction system of the collection is discrete and contains:
a single molecule of a template nucleic acid whose base sequence includes a first target sequence element and a second target sequence element, wherein the first and second target sequence elements are separated from one another in the template nucleic acid by a spacer sequence element that comprises a plurality of residues, and further wherein at least one of the target sequence elements is present in two or more different forms within the set of template nucleic acids present in the collection; and
reagents for amplifying nucleic acids;
under conditions and for a time sufficient that, within each discrete reaction system, a product nucleic acid in which the first and second target sequences, or the complement sequences thereof, are linked to one another and some or all of the residues of the spacer element, or the complement thereof, are absent.

20. A method of phasing allelic variants of a first and a second genetic locus on a chromosome or chromosomal fragment comprising the first and the second genetic locus, the method comprising the steps of:
(a) providing a sample comprising one or more haplotypes of the chromosome or chromosomal fragment;
(b) diluting and partitioning the sample into aliquots so that a plurality of the aliquots contain no more than one haplotype of the chromosome or chromosomal fragment;
(c) generating a plurality of amplicons from at least one chromosome or chromosomal fragment, wherein the amplicons are generated by polymerase chain reaction in the presence of a first and second pair of primers for the first and second genetic locus, respectively, each pair comprising a forward and a reverse primer, and wherein one of the first pair comprises a region of complementarity to one of the second pair, and wherein one or more of the amplicons comprises the first and the second genetic locus; and
(d) determining the allelic variants of the first and second genetic loci on the one or more of the amplicons comprising the first and the second genetic locus to determine the phasing of the allelic variants of the first and second genetic locus on the chromosome or chromosomal fragment.

21. A method of phasing allelic variants of a plurality of genetic loci on a nucleic acid template comprising the plurality of loci, the method comprising the steps of:
(a) providing a sample comprising one or more types of the nucleic acid template;
(b) partitioning the sample into aliquots so that a plurality of the aliquots contain no more than one type of the template;
(c) generating, from each aliquot containing one type of the template, a plurality of nucleic acid polymers based on the template, wherein at least one of the nucleic acid polymers comprises each of the plurality of genetic loci; and
(d) sequencing one or more of the nucleic acid polymers comprising each of the genetic loci to determine the phasing of the allelic variants of the plurality of genetic loci.

22. The method or composition of any one of the preceding claims, wherein the first and/or second genetic locus or sequence element independently comprises a SNP, a RFLP, an AFLP, an isozyme, a SSR, a mutation, a genetic lesion, a repeat expansion, a deletion, a truncation, an insertion, an inversion, fusion, or a region of chromosome breakage and/or chromosome breakage and/or re-attachment.

23. The method or composition of any one of the preceding claims, wherein the first and second genetic locus or sequence element is at least 20, 30, 40, 50, 60, 70, 80, 90, 100, 150, 200, 300, 400, or 500 bp in length.

24. The method or composition of any one of the preceding claims, wherein the first and/or second genetic locus or sequence element are selected from: a SNP, a RFLP, an AFLP, an isozyme, a SSR, a mutation, a genetic lesion, a SNP, a repeat expansion, a deletion, a truncation, an insertion, an inversion, fusion, or a region of chromosome breakage and/or chromosome breakage and/or re-attachment.

25. The method or composition of any one of the preceding claims, wherein the repeat expansion is an expansion of a trinucleotide, tetranucleotide, or hexanucleotide repeat.

26. The method or composition of any one of the preceding claims, wherein the repeat expansion is associated with a disorder.

27. The method or composition of any one of the preceding claims, wherein the repeat expansion is associated with a disorder selected from: neurological disorder, Huntington's disease, fragile X syndrome, fragile X-E syndrome, fragile X-associated tremor/ataxia syndrome, dystrophy, muscular dystrophy, myotonic dystrophy, juvenile myoclonic epilepsy, ataxia, Friedreich's ataxia, spinocerebellar ataxia, atrophy, spino-bulbar muscular atrophy, Dentatorubropallidoluysian atrophy, ALS, frontotemporal lobar degeneration, frontotemporal dementia, and asthma.

28. The method or composition of any one of the preceding claims, wherein the sample is from a patient afflicted with, showing symptoms of, suspected to have, having or suspected of having a genetic predisposition to, or susceptible to a disorder selected from cancer, autoimmune disease, infection, neurological, neuromuscular or neurodegenerative disease, and wherein the first and/or second genetic locus is associated with the disorder.

29. The method or composition of any one of the preceding claims, wherein the sample is from a patient afflicted with, showing symptoms of, suspected to have, having or suspected of having a genetic predisposition to, or susceptible to a cancer.

30. The method or composition of any one of the preceding claims, wherein the sample is from a patient afflicted with, showing symptoms of, suspected to have, having or suspected of having a genetic predisposition to, or susceptible to an autoimmune disease.

31. The method or composition of any one of the preceding claims, wherein the sample is from a patient afflicted with, showing symptoms of, suspected to have, having or suspected of having a genetic predisposition to, or susceptible to an infection.

32. The method or composition of any one of the preceding claims, wherein the first and second genetic loci or sequence element are on the same gene.

33. The method or composition of any one of the preceding claims, wherein the first and second genetic loci or sequence element are on different genes.

34. The method or composition of any one of the preceding claims, wherein the first spacing region is longer than the second spacing region.

35. The method or composition of any one of the preceding claims, wherein the length of the first spacing region is at least 0.01, 0.025, 0.05, 0.075, 0.1, 0.25, 0.5, 0.75, 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, 1000 kb.

36. The method or composition of any one of the preceding claims, wherein the second spacing region is no more than 10, 25, 50, 100, 150, 200, 225, 250, 300, 350, 400, 450, 500 bp.

37. The method or composition of any one of the preceding claims, wherein the nucleic acid template is selected from: a chromosome or fragment thereof, genomic DNA, mRNA and cDNA.

38. The method or composition of any one of the preceding claims, wherein the sample is selected from: tissue, cells, blood, sputum, cheek swab, urine, FFPE, and a prepared sample.

39. The method or composition of any one of the preceding claims, wherein the method or composition further comprises the step of diluting the sample prior to partitioning the sample into aliquots.

40. The method or composition of any one of the preceding claims, wherein aliquots are selected from: droplets, microdroplets, droplets in an emulsion, aqueous droplets in oil, aliquots on a solid surface, and aliquots in wells covered with oil.

41. The method or composition of any one of the preceding claims, wherein the oil comprises a fluorinated oil, a surfactant, and/or a fluorosurfactant.

42. The method or composition of any one of the preceding claims, wherein the one or more types are haplotypes.

43. The method or composition of any one of the preceding claims, wherein the nucleic acid polymers are amplicons generated by polymerase chain reaction.

44. The method or composition of any one of the preceding claims, wherein the nucleic acid polymers are amplicons generated by polymerase chain reaction in the presence of a first and second pair of primers for the first and second genetic loci, respectively, each pair comprising a forward and a reverse primer, and wherein one of the first pair comprises a region of complementarity with one of the second pair, wherein the region of complementarity comprises the second spacing region, or the second spacing region comprises the region of complementarity.

45. The method or composition of any one of the preceding claims, wherein the distance between the first genetic locus and either of the first pair of primers is no more than 10, 25, 50, 100, 150, 200, 225, 250, 300, 350, 400, 450, or 500 bp, and/or the distance between the second genetic locus and either of the second pair of primers is no more than 10, 25, 50, 100, 150, 200, 225, 250, 300, 350, 400, 450, or 500 bp.

46. The method or composition of any one of the preceding claims, wherein the region of complementarity is GC-rich.

47. The method or composition of any one of the preceding claims, wherein the region of complementarity is at least 5, 10, 15, 20, 25, 30, 35, 40, 45, 50 nt long.

48. The method or composition of any one of the preceding claims, wherein the step of phasing the allelic variants is performed using a method or composition selected from: sequencing, hybridization with probes, hybridization with allele-specific probes, and amplification with allele-specific probes.

49. The method or composition of any one of the preceding claims, wherein the step of phasing the allelic variants further comprises the step of barcoding the one or more of the nucleic acid polymers prior to sequencing the nucleic acid polymers.

50. The method or composition of any one of the preceding claims, wherein of phasing the allelic variants further comprises the steps of combining two or more of the aliquots and barcoding the one or more of the nucleic acid polymers prior to sequencing the nucleic acid polymers, wherein the steps of combining two or more of the aliquots and barcoding the one or more of the nucleic acid polymers can be performed in either order.

51. The method or composition of any one of the preceding claims, wherein, in the step of phasing the allelic variants, sequencing is performed using a next generation sequencing technique.

52. The method or composition of any one of the preceding claims, wherein the organism or individual, the one or more types of nucleic acid templates, or the sample is heterozygous or compound heterozygous at one or more of the first, second or multiple genetic loci.

53. The method or composition of any one of the preceding claims, wherein the nucleic acid template is determined to be heterozygous at the genetic loci.

54. The method or composition of any one of the preceding claims, wherein the nucleic acid template is determined to be heterozygous at the genetic loci, wherein the determination is performed using an PCR based genotyping assay.

55. The method or composition of any one of claims 1, 2 or 5, wherein the nucleic acid template in step (a) has been determined to be heterozygous at the genetic loci.

56. The method or composition of any one of claims 1, 2 or 5, wherein the nucleic acid template in step (a) has been determined to be heterozygous at the genetic loci using an PCR based genotyping assay.

57. The method or composition of any one of the preceding claims, wherein the length of the second spacing region is at least 0.01, 0.025, 0.05, 0.075, 0.1, 0.25, 0.5, 0.75, 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, 1000 kb shorter than that of the first spacing region.

58. The method or composition of any one of the preceding claims, wherein the length of the second spacing region is at least 1, 2, 5, 10, 20, 30, 40, 50, 60, 70, 80, 90, 100, 125, 150, 175, 200, 225, 250, 275, 300, 325, 350, 375, 400, 425, 450, 500, 550, 600, 650, 700, 750, 800, 850, 900, 950, 1000 kb shorter than that of the first spacing region.

59. The method or composition of any one of the preceding claims, wherein the length of the second spacing region is at least 5 kb shorter than that of the first spacing region.

60. The method or composition of any one of the preceding claims, wherein the length of the second spacing region is at least 10 kb shorter than that of the first spacing region.

61. The method or composition of any one of the preceding claims, wherein the first genetic locus or sequence element comprises a SNP in Huntingtin.

62. The method or composition of any one of the preceding claims, wherein the first genetic locus or sequence element comprises a SNP in Huntingtin selected from rs362267, rs6844859, rs1065746, rs7685686, rs362331, rs362336, rs2024115, rs362275, rs362273, rs362272, rs3025805, rs3025806, rs35892913, rs363125, rs17781557, rs4690072, rs4690074, rs1557210, rs363088, rs362268, rs362308, rs362307, rs362306, rs362305, rs362304, rs362303, rs362302, rs363075, rs2530595, and rs2298969.

63. The method or composition of any one of the preceding claims, wherein the first genetic locus or sequence element comprises SNP rs362307.

64. The method or composition of any one of claim 62, wherein the first genetic locus or sequence element comprises SNP rs2530595.

65. The method or composition of any one of claim 62, wherein the first genetic locus or sequence element comprises SNP rs362331.

66. The method or composition of any one of the preceding claims, wherein the second genetic locus or sequence element comprises CAG repeats in Huntingtin.

67. The method or composition of any one of the preceding claims, wherein the second genetic locus or sequence element comprises at least 27 CAG repeats in Huntingtin.

68. The method or composition of any one of the preceding claims, wherein the second genetic locus or sequence element comprises at least 36 CAG repeats in Huntingtin.

69. The method or composition of any one of the preceding claims, wherein the second genetic locus or sequence element comprises at least 40 CAG repeats in Huntingtin.


SEQUENCE LISTING (58 sequences)

SEQ ID NO: 1 - DNA, 20 bp, Artificial Sequence
  Description of Artificial Sequence Synthetic oligonucleotide
gcctcagtctgcttcgcacc

SEQ ID NO: 2 - DNA, 20 bp, Artificial Sequence
  Description of Artificial Sequence Synthetic oligonucleotide
gcctcagtctgcttcgcacc

SEQ ID NO: 3 - DNA, 28 bp, Artificial Sequence
  Description of Artificial Sequence Synthetic primer
tttgtgacccacgcctgctccctcagct

SEQ ID NO: 4 - DNA, 29 bp, Artificial Sequence
  Description of Artificial Sequence Synthetic primer
ccggcctccaggatgaagtgcacacattg

SEQ ID NO: 5 - DNA, 28 bp, Artificial Sequence
  Description of Artificial Sequence Synthetic primer
tcccgctgagtctggatctccaggcagg

SEQ ID NO: 6 - DNA, 28 bp, Artificial Sequence
  Description of Artificial Sequence Synthetic primer
gatggggcccagcccttcctgatgcata

SEQ ID NO: 7 - DNA, 27 bp, Artificial Sequence
  Description of Artificial Sequence Synthetic primer
agcagctgtggtcccgggtcctccacg

SEQ ID NO: 8 - DNA, 27 bp, Artificial Sequence
  Description of Artificial Sequence Synthetic primer
tgtccctcccccgcttcctccctcggt

SEQ ID NO: 9 - DNA, 28 bp, Artificial Sequence
  Description of Artificial Sequence Synthetic primer
gagcggcccctgaaccttccaggttgcc

SEQ ID NO: 10 - DNA, 28 bp, Artificial Sequence
  Description of Artificial Sequence Synthetic primer
taactcggtgtgtggccgcctggcaggt

SEQ ID NO: 11 - DNA, 26 bp, Artificial Sequence
  Description of Artificial Sequence Synthetic primer
ctcggtggaggcagggcacaaggtcg

SEQ ID NO: 12 - DNA, 27 bp, Artificial Sequence
  Description of Artificial Sequence Synthetic primer
ctggggccggagcctttggaagtcggt

SEQ ID NO: 13 - DNA, 28 bp, Artificial Sequence
  Description of Artificial Sequence Synthetic primer
cccaccaggactgcagacactccctgcc

SEQ ID NO: 14 - DNA, 28 bp, Artificial Sequence
  Description of Artificial Sequence Synthetic primer
agctcgaccgcagggccttccagtctgt

SEQ ID NO: 15 - DNA, 20 bp, Artificial Sequence
  Description of Artificial Sequence Synthetic primer
tttttttttttttttttttt

SEQ ID NO: 16 - DNA, 19 bp, Artificial Sequence
  Description of Artificial Sequence Synthetic primer
ggcgaccctggaaaagctg

SEQ ID NO: 17 - DNA, 23 bp, Artificial Sequence
  Description of Artificial Sequence Synthetic primer
atggcgaccctggaaaagctgat

SEQ ID NO: 18 - DNA, 43 bp, Artificial Sequence
  Description of Artificial Sequence Synthetic primer
cactgtcatgccgttacgtagcggacaatgattcacacggtct

SEQ ID NO: 19 - DNA, 41 bp, Artificial Sequence
  Description of Artificial Sequence Synthetic primer
cactgtcatgccgttacgtagcgtgaggcagcagcggctgt

SEQ ID NO: 20 - DNA, 49 bp, Artificial Sequence
  Description of Artificial Sequence Synthetic primer
cgctacgtaacggcatgacagtgcgaaatgtccacaaggtcaccacctg

SEQ ID NO: 21 - DNA, 47 bp, Artificial Sequence
  Description of Artificial Sequence Synthetic primer
cgctacgtaacggcatgacagtggccatggtgggagagactgtgagg

SEQ ID NO: 22 - DNA, 26 bp, Artificial Sequence
  Description of Artificial Sequence Synthetic primer
atagggaccaagctggctcggtggag

SEQ ID NO: 23 - DNA, 26 bp, Artificial Sequence
  Description of Artificial Sequence Synthetic primer
atgtgcggaagcccatagggaccaag

SEQ ID NO: 24 - DNA, 52 bp, Artificial Sequence
  Description of Artificial Sequence Synthetic primer
tcgtcggcagcgtcagatgtgtataagagacagggcgaccctggaaaagctg

SEQ ID NO: 25 - DNA, 60 bp, Artificial Sequence
  Description of Artificial Sequence Synthetic primer
gtctcgtgggctcggagatgtgtataagagacagatagggaccaagctggctcggtggag

SEQ ID NO: 26 - DNA, 60 bp, Artificial Sequence
  Description of Artificial Sequence Synthetic primer
gtctcgtgggctcggagatgtgtataagagacagatgtgcggaagcccatagggaccaag

SEQ ID NO: 27 - DNA, 48 bp, Artificial Sequence
  Description of Artificial Sequence Synthetic primer
cgctacgtaacggcatgacagtgggagcgtggtctcctccacagagtt

SEQ ID NO: 28 - DNA, 48 bp, Artificial Sequence
  Description of Artificial Sequence Synthetic primer
cgctacgtaacggcatgacagtgagcagacatcctcatcgggctttgt

SEQ ID NO: 29 - DNA, 49 bp, Artificial Sequence
  Description of Artificial Sequence Synthetic primer
cgctacgtaacggcatgacagtgagccgactctccaactgaaagaggtg

SEQ ID NO: 30 - DNA, 25 bp, Artificial Sequence
  Description of Artificial Sequence Synthetic primer
tcgctgatggcttttggggtatttg

SEQ ID NO: 31 - DNA, 25 bp, Artificial Sequence
  Description of Artificial Sequence Synthetic primer
aagaagctgctctccaggctgcact

SEQ ID NO: 32 - DNA, 25 bp, Artificial Sequence
  Description of Artificial Sequence Synthetic primer
gcctccaggatgaagtgcacacagt

SEQ ID NO: 33 - DNA, 25 bp, Artificial Sequence
  Description of Artificial Sequence Synthetic primer
cctacacgtgacccctctggaggac

SEQ ID NO: 34 - DNA, 25 bp, Artificial Sequence
  Description of Artificial Sequence Synthetic primer
tcctgagctctccaaggtccctcag

SEQ ID NO: 35 - DNA, 25 bp, Artificial Sequence
  Description of Artificial Sequence Synthetic primer
gcttcagaatttggagcattttgga

SEQ ID NO: 36 - DNA, 27 bp, Artificial Sequence
  Description of Artificial Sequence Synthetic primer
aagcttcagaatttggagcattttgga

SEQ ID NO: 37 - DNA, 61 bp, Artificial Sequence
  Description of Artificial Sequence Synthetic primer
ctactccttcagtccatgtcagtgtcctcgtgctccagtcggacaatgattcacacggtct

SEQ ID NO: 38 - DNA, 59 bp, Artificial Sequence
  Description of Artificial Sequence Synthetic primer
ctactccttcagtccatgtcagtgtcctcgtgctccagtcgtgaggcagcagcggctgt

SEQ ID NO: 39 - DNA, 65 bp, Artificial Sequence
  Description of Artificial Sequence Synthetic primer
cgactggagcacgaggacactgacatggactgaaggagtaggccatggtgggagagactgtgagg

SEQ ID NO: 40 - DNA, 66 bp, Artificial Sequence
  Description of Artificial Sequence Synthetic primer
cgactggagcacgaggacactgacatggactgaaggagtagagcagacatcctcatcgggctttgt

SEQ ID NO: 41 - DNA, 66 bp, Artificial Sequence
  Description of Artificial Sequence Synthetic primer
cgactggagcacgaggacactgacatggactgaaggagtagggagcgtggtctcctccacagagtt

SEQ ID NO: 42 - DNA, 67 bp, Artificial Sequence
  Description of Artificial Sequence Synthetic primer
cgactggagcacgaggacactgacatggactgaaggagtagagccgactctccaactgaaagaggtg

SEQ ID NO: 43 - DNA, 59 bp, Artificial Sequence
  Description of Artificial Sequence Synthetic primer
gtctcgtgggctcggagatgtgtataagagacagcctacacgtgacccctctggaggac

SEQ ID NO: 44 - DNA, 59 bp, Artificial Sequence
  Description of Artificial Sequence Synthetic primer
gtctcgtgggctcggagatgtgtataagagacagtcctgagctctccaaggtccctcag

SEQ ID NO: 45 - DNA, 59 bp, Artificial Sequence
  Description of Artificial Sequence Synthetic primer
gtctcgtgggctcggagatgtgtataagagacagtcgctgatggcttttggggtatttg

SEQ ID NO: 46 - DNA, 59 bp, Artificial Sequence
  Description of Artificial Sequence Synthetic primer
gtctcgtgggctcggagatgtgtataagagacagaagaagctgctctccaggctgcact

SEQ ID NO: 47 - DNA, 59 bp, Artificial Sequence
  Description of Artificial Sequence Synthetic primer
gtctcgtgggctcggagatgtgtataagagacaggcctccaggatgaagtgcacacagt

SEQ ID NO: 48 - DNA, 61 bp, Artificial Sequence
  Description of Artificial Sequence Synthetic primer
gtctcgtgggctcggagatgtgtataagagacagaagcttcagaatttggagcattttgga

SEQ ID NO: 49 - DNA, 59 bp, Artificial Sequence
  Description of Artificial Sequence Synthetic primer
gtctcgtgggctcggagatgtgtataagagacaggcttcagaatttggagcattttgga

SEQ ID NO: 50 - DNA, 151 bp, Homo sapiens
ggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcct

SEQ ID NO: 51 - DNA, 151 bp, Homo sapiens
ggcgaccctggaaaagctgatgaaggccttcgagtccctcaagtccttccagcagcagcagcagcagcagcagcagcagcagcagcagcagcagcagcagcagcagcagcagcagcagcagcagcagcagcagcagcagcagcagcagcag

SEQ ID NO: 52 - DNA, 151 bp, Homo sapiens
atagggaccaagctggctcggtggaggcagggcacaagggcacagacttccaaaggctccggccccagctgccgcctcacagtctctcccaccatggccactgtcatgccgttacgtagcgtgaggcagcagcggctgtgcctgctgctgc

SEQ ID NO: 53 - DNA, 150 bp, Homo sapiens
atagggaccaagctggctcggtggaggcagggcacaagggcgcagacttccaaaggctccggccccagctgccgcctcacagtctctcccaccatggccactgtcatgccgttacgtagcgtgaggcagcagcggctgtagttccatgca

SEQ ID NO: 54 - DNA, 63 bp, Homo sapiens
cagcagcagcagcagcagcagcagcagcagcagcagcagcagcagcagcagcagcagcagcag

SEQ ID NO: 55 - DNA, 198 bp, Homo sapiens
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 56 - DNA, 252 bp, Homo sapiens
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 57 - DNA, 102 bp, Homo sapiens
cagcagcagcagcagcagcagcagcagcagcagcagcagcagcagcagcagcagcagcagcagcagcagcagcagcagcagcagcagcagcagcagcagcag

SEQ ID NO: 58 - DNA, 16 bp, Artificial Sequence
  Description of Artificial Sequence Synthetic oligonucleotide
tcctgagctctctccg